SEQUENCE LISTING (80 sequences)

SEQ ID NO: 1 - DNA, 405 bp, Sus scrofa
atggccgaacagtccgacaaagccgtgaagtattacaccctggaagagatccagaagcacaacaacagcaagagcacctggctaatcctgcaccacaaagtgtacgatttgaccaaatttttggaggagcatcctggtggggaagaagtcttaagggaacaagctggaggtgatgctactgaaaattttgaggatgttggacactccacagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 2 - protein, 134 aa, Sus scrofa
MAEQSDKAVKYYTLEEIQKHNNSKSTWLILHHKVYDLTKFLEEHPGGEEVLREQAGGDATENFEDVGHSTDARELSKTFIIGELHPDDRSKIAKPSETLITTVESNSSWWTNWVIPAISALVVSLMYHFYTSEN

SEQ ID NO: 3 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 4 - protein, 509 aa, Sus scrofa
MWVLLVFFLLTLTYLFWPKTKGSGAKYPRSLPVLPVVGSLPFLPRRGHQHMNFFKLQDKYGPIFSFRLGSKTTVVIGDHQLAKEVLLKKGKEFSGRPRVMTLDILSDNQKGIAFADHGTSWQLHRKLALSTFSLFKGGNLKLENIINQEIKVLCDFLATRNGESIDLAQPLSLAMTNIVSFICFNFSFKKGDPALQAIVNFNDGILDAVGKEILYDMFPGIRILPSQTLENMKQCVRMRNELLREILENRKENYSRNSITNLLDIMIQAKTNAESNTGGPDHNLKLLSDRHMLATVADIFGAGVETSASVVKWIVAFLLHYPLLRKKIQDAIDQNIGFNRAPSISDRNQLVLLEATIREVLRFRPVSPTLIPHRAIIDSSIGEFTIDKDTDVVVNLWALHHNEKEWLRPDLFMPERFLDPTGTQLISPSLSYLPFGAGPRSCVGEMLARQELFLFTAGLLQRFDLELPDDGQLPCLVGNPSLVLQIDPFKVKIKERQAWKEAHTEGSTS

SEQ ID NO: 5 - protein, 44 aa, Homo sapiens
QLAKEVLIKKGKDFSGRPQMATLDIASNNRKGIAFADSGAHWQL

SEQ ID NO: 6 - protein, 44 aa, Sus scrofa
QLAKEVLLKKGKEFSGRPRVMTLDILSDNQKGIAFADHGTSWQL

SEQ ID NO: 7 - protein, 44 aa, Rattus norvegicus
QLAREVLIKKGKEFSGRPQMVTQSLLSDQGKGVAFADAGSSWHL

SEQ ID NO: 8 - protein, 30 aa, Homo sapiens
RTPTISDRNRLLLLEATIREVLRLRPVAPM

SEQ ID NO: 9 - protein, 30 aa, Sus scrofa
RAPSISDRNQLVLLEATIREVLRFRPVSPT

SEQ ID NO: 10 - protein, 30 aa, Rattus norvegicus
RTPTFNDRSHLLMLEATIREVLRIRPVAPM

SEQ ID NO: 11 - protein, 30 aa, Homo sapiens
RSCIGEALARQELFLIMAWLLQRFDFDVSD

SEQ ID NO: 12 - protein, 30 aa, Sus scrofa
RSCVGEMLARQELFLFTAGLLQRFDLELPD

SEQ ID NO: 13 - protein, 30 aa, Rattus norvegicus
RSCIGEALARQELFVFTALLLQRFDLDVSD

SEQ ID NO: 14 - protein, 32 aa, Sus scrofa
EHPGGEEVLREQAGGDATENFEDVGHSTDARE

SEQ ID NO: 15 - protein, 32 aa, Sus scrofa
EHPGGEEVLMEQAGRDATESFEDVGHSSDARE

SEQ ID NO: 16 - protein, 50 aa, Rattus norvegicus
MAEQSDKDVKYYTLEEIQKHKDSKSTWVILHHKVYDLTKFLEEHPGGEEV

SEQ ID NO: 17 - protein, 50 aa, Homo sapiens
MAEQSDEAVKYYTLEEIQKHNHSKSTWLILHHKVYDLTKFLEEHPGGEEV

SEQ ID NO: 18 - protein, 50 aa, Sus scrofa
MAEQSDKAVKYYTLEEIQKHNNSKSTWLILHHKVYDLTKFLEEHPGGEEV

SEQ ID NO: 19 - protein, 508 aa, Homo sapiens
MWELVALLLLTLAYLFWPKRRCPGAKYPKSLLSLPLVGSLPFLPRHGHMHNNFFKLQKKYGPIYSVRMGTKTTVIVGHHQLAKEVLIKKGKDFSGRPQMATLDIASNNRKGIAFADSGAHWQLHRRLAMATFALFKDGDQKLEKIICQEISTLCDMLATHNGQSIDISFPVFVAVTNVISLICFNTSYKNGDPELNVIQNYNEGIIDNLSKDSLVDLVPWLKIFPNKTLEKLKSHVKIRNDLLNKILENYKEKFRSDSITNMLDTLMQAKMNSDNGNAGPDQDSELLSDNHILTTIGDIFGAGVETTTSVVKWTLAFLLHNPQVKKKLYEEIDQNVGFSRTPTISDRNRLLLLEATIREVLRLRPVAPMLIPHKANVDSSIGEFAVDKGTEVIINLWALHHNEKEWHQPDQFMPERFLNPAGTQLISPSVSYLPFGAGPRSCIGEILARQELFLIMAWLLQRFDLEVPDDGQLPSLEGIPKVVFLIDSFKVKIKVRQAWREAQAEGST

SEQ ID NO: 20 - protein, 507 aa, Rattus norvegicus
MWELVGLLLLILAYFFWVKSKTPGAKLPRSLPSLPLVGSLPFLPRRGHMHVNFFKLQEKYGPIYSLRLGTTTTVIIGHYQLAREVLIKKGKEFSGRPQMVTQSLLSDQGKGVAFADAGSSWHLHRKLVFSTFSLFKDGQKLEKLICQEAKSLCDMMLAHDKESIDLSTPIFMSVTNIICAICFNISYEKNDPKLTAIKTFTEGIVDATGDRNLVDIFPWLTIFPNKGLEVIKGYAKVRNEVLTGIFEKCREKFDSQSISSLTDILIQAKMNSDNNNSCEGRDPDVFSDRHILATVGDIFGAGIETTTTVLKWILAFLVHNPEVKKKIQKEIDQYVGFSRTPTFNDRSHLLMLEATIREVLRIRPVAPMLIPHKANVDSSIGEFTVPKDTHVVVNLWALHHDENEWDQPDQFMPERFLDPTGSHLITPTQSYLPFGAGPRSCIGEALARQELFVFTALLLQRFDLDVSDDKQLPRLEGDPKVVFLIDPFKVKITVRQAWMDAQAEVST

SEQ ID NO: 21 - protein, 134 aa, Homo sapiens
MAEQSDEAVKYYTLEEIQKHNHSKSTWLILHHKVYDLTKFLEEHPGGEEVLREQAGGDATENFEDVGHSTDAREMSKTFIIGELHPDDRPKLNKPPETLITTIDSSSSWWTNWVIPAISAVAVALMYRLYMAED

SEQ ID NO: 22 - protein, 134 aa, Rattus norvegicus
MAEQSDKDVKYYTLEEIQKHKDSKSTWVILHHKVYDLTKFLEEHPGGEEVLREQAGGDATENFEDVGHSTDARELSKTYIIGELHPDDRSKIAKPSETLITTVESNSSWWTNWVIPAISALVVALMYRLYMAED

SEQ ID NO: 23 - DNA, 405 bp, sus scrofa
atggccgaacagtccgacaaagccgtgaagtattacaccctggaagagatccagaagcacaagaacagcaagagcacctggctaatcctgcaccacaaagtgtacgatttgaccaaatttttggaggagcatcctggtggggaagaagtcttaagggaacaagctggaggtgatgctactgaaaattttgaggatgttggacactccacagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 24 - DNA, 405 bp, Sus scrofa
atggccgaacagtccgacaaagccgtgaagtattacaccctggaagagatccagaagcacaacaacagcaagagcacctgggtaatcctgcaccacaaagtgtacgatttgaccaaatttttggaggagcatcctggtggggaagaagtcttaagggaacaagctggaggtgatgctactgaaaattttgaggatgttggacactccacagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 25 - DNA, 405 bp, Sus scrofa
atggccgaacagtccgacaaagccgtgaagtattacaccctggaagagatccagaagcacaacaacagcaagagcacctggctaatcctgcaccacaaagtgtacgatttgaccaaatttttggaggagcatcctggtggggaagaagtcttaatggaacaagctggaggtgatgctactgaaaattttgaggatgttggacactccacagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 26 - DNA, 405 bp, Sus scrofa
atggccgaacagtccgacaaagccgtgaagtattacaccctggaagagatccagaagcacaacaacagcaagagcacctggctaatcctgcaccacaaagtgtacgatttgaccaaatttttggaggagcatcctggtggggaagaagtcttaagggaacaagctggacgtgatgctactgaaaattttgaggatgttggacactccacagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 27 - DNA, 405 bp, Sus scrofa
atggccgaacagtccgacaaagccgtgaagtattacaccctggaagagatccagaagcacaacaacagcaagagcacctggctaatcctgcaccacaaagtgtacgatttgaccaaatttttggaggagcatcctggtggggaagaagtcttaagggaacaagctggaggtgatgctactgaaagttttgaggatgttggacactccacagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 28 - DNA, 405 bp, Sus scrofa
atggccgaacagtccgacaaagccgtgaagtattacaccctggaagagatccagaagcacaacaacagcaagagcacctggctaatcctgcaccacaaagtgtacgatttgaccaaatttttggaggagcatcctggtggggaagaagtcttaagggaacaagctggaggtgatgctactgaaaattttgaggatgttggacactcctcagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 29 - DNA, 405 bp, Sus scrofa
atggccgaacagtccgacaaagccgtgaagtattacaccctggaagagatccagaagcacaagaacagcaagagcacctgggtaatcctgcaccacaaagtgtacgatttgaccaaatttttggaggagcatcctggtggggaagaagtcttaagggaacaagctggaggtgatgctactgaaaattttgaggatgttggacactccacagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 30 - DNA, 405 bp, Sus scrofa
atggccgaacagtccgacaaagccgtgaagtattacaccctggaagagatccagaagcacaacaacagcaagagcacctggctaatcctgcaccacaaagtgtacgatttgaccaaatttttggaggagcatcctggtggggaagaagtcttaatggaacaagctggaggtgatgctactgaaagttttgaggatgttggacactccacagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 31 - DNA, 405 bp, Sus scrofa
atggccgaacagtccgacaaagccgtgaagtattacaccctggaagagatccagaagcacaacaacagcaagagcacctggctaatcctgcaccacaaagtgtacgatttgaccaaatttttggaggagcatcctggtggggaagaagtcttaatggaacaagctggacgtgatgctactgaaagttttgaggatgttggacactcctcagatgctcgagagttgtccaaaacgttcatcattggggagctgcatccggatgacagatcaaagattgccaagccttcggaaactcttattaccactgttgaatctaattccagctggtggaccaactgggtgatcccagccatctcagcactggttgtatccctgatgtatcacttctacacatcggaaaactaa

SEQ ID NO: 32 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactcaagacatcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 33 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctatccatcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 34 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacctcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 35 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctggcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 36 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctggacgacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 37 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctgtcagaccagggaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 38 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctgtcagacaaccaaaagggggttgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 39 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcactgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 40 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctttcaacgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 41 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggagccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 42 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcatgctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 43 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctagacatcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcgtcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 44 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactcaagacatcctgtcagacaaccaaaagggggttgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 45 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactcaatccatcctgtcagacaaccaaaagggggttgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 46 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactcaatccctcctgtcagaccagggaaagggggttgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 47 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctatccatcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcgtcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 48 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctatccctcctgtcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcgtcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 49 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactctatccatcctggcagacaaccaaaaggggattgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcgtcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 50 - DNA, 1530 bp, Sus scrofa
atgtgggtgctcttggttttcttcttgctcaccctcacctatttattttggcctaagaccaagggctctggtgccaagtaccccaggagtctcccagtcctgcccgtggtgggcagcctgccattcctacccagacgtggccaccagcacatgaacttcttcaagttgcaggacaaatatggccccatcttctcctttcgtctgggttccaagactaccgtggtaattggtgaccaccagctggccaaggaggtgcttctcaagaagggcaaggaattctccgggcggcccagagtgatgactcaatccatcctggcagacaaccaaaagggggttgccttcgccgaccatggtacctcctggcagctgcatcggaagctggcactgagcaccttttccctgttcaagggtggcaacctgaagctggagaacatcattaatcaagaaatcaaagtactgtgcgatttcctggccacacggaatggagagtccattgatttggcccagcctctctctctggcgatgaccaacatagtcagctttatctgcttcaacttctccttcaagaagggggatcccgcgctgcaggccatagtgaatttcaatgatggcatcctggatgctgtgggcaaggaaattttgtatgacatgttccctggaattaggattttacccagccaaactctggaaaacatgaagcagtgtgttagaatgcgaaacgaattgctgcgggaaatccttgaaaaccgtaaggagaactacagcagaaactccatcactaacttgttggacataatgatccaagccaagacgaacgcagaaagtaacactggtggcccagaccacaatttaaagctgctttcagacagacacatgctcgccactgttgcggacatctttggggccggtgtggagacttctgcctctgtggtaaagtggatcgtggccttcctgctacactatcctctgctgaggaagaagatccaggatgctatcgaccagaatattggtttcaatcgtgccccatctatcagcgaccggaaccaacttgtcctcctggaggccaccatccgagaggtgcttcgattccggcctgtgtcccctacgctcatcccccacagggctatcattgactccagcattggcgaatttaccattgacaaggacacagatgtcgtcgtcaatctgtgggcactgcatcacaatgagaaggagtggctccggcccgacctgttcatgcctgagcgcttcctggaccccacgggaacccagctcatctcaccatcattgagctacttgcccttcggagcaggaccccgctcttgcgtaggggagatgctagcccgccaggagctcttcctcttcacggctggattgctgcagaggttcgacctggagctcccagatgatgggcagctaccctgtctcgtgggcaaccccagtttggtcctgcagatagatcctttcaaagtgaagatcaaggagcgccaggcctggaaggaagcccacactgaggggagtacctcctga

SEQ ID NO: 51 - DNA, 30 bp, Sus scrofa
ccagagtgatgactcaagacatcctgtcag

SEQ ID NO: 52 - DNA, 35 bp, Sus scrofa
cagagtgatgactctatccatcctgtcagacaacc

SEQ ID NO: 53 - DNA, 30 bp, Sus scrofa
gtgatgactctagacctcctgtcagacaac

SEQ ID NO: 54 - DNA, 41 bp, Sus scrofa
ctagacatcctgtcagaccagggaaaggggattgccttcgc

SEQ ID NO: 55 - DNA, 30 bp, Sus scrofa
gacaaccaaaagggggttgccttcgccgac

SEQ ID NO: 56 - DNA, 35 bp, Sus scrofa
cagagtgatgactcaatccatcctgtcagacaacc

SEQ ID NO: 57 - DNA, 41 bp, Sus scrofa
caatccatcctgtcagaccagggaaagggggttgccttcgc

SEQ ID NO: 58 - DNA, 30 bp, Sus scrofa
gtgatgactcaatccctcctgtcagaccag

SEQ ID NO: 59 - DNA, 41 bp, Sus scrofa
gatgactctagacatcctggacgacaaccaaaaggggattg

SEQ ID NO: 60 - DNA, 33 bp, Sus scrofa
actctagacatcctggcagacaaccaaaagggg

SEQ ID NO: 61 - DNA, 33 bp, Sus scrofa
ccccttttggttgtctgccaggatgtctagagt

SEQ ID NO: 62 - DNA, 30 bp, Sus scrofa
ccatagtgaatttcactgatggcatcctgg

SEQ ID NO: 63 - DNA, 44 bp, Sus scrofa
gtttcaatcgtgccccatctttcaacgaccggaaccaacttgtc

SEQ ID NO: 64 - DNA, 30 bp, Sus scrofa
ctatcagcgaccggagccaacttgtcctcc

SEQ ID NO: 65 - DNA, 33 bp, Sus scrofa
cggaaccaacttgtcatgctggaggccaccatc

SEQ ID NO: 66 - DNA, 25 bp, Sus scrofa
caggagctcttcgtcttcacggctg

SEQ ID NO: 67 - DNA, 41 bp, Sus scrofa
gatgactcaatccatcctggcagacaaccaaaagggggttg

SEQ ID NO: 68 - DNA, 41 bp, Sus scrofa
caacccccttttggttgtctgccaggatggattgagtcatc

SEQ ID NO: 69 - DNA, 41 bp, Sus scrofa
gatgactctatccatcctggcagacaaccaaaaggggattg

SEQ ID NO: 70 - DNA, 41 bp, Sus scrofa
caatccccttttggttgtctgccaggatggatagagtcatc

SEQ ID NO: 71 - DNA, 30 bp, Sus scrofa
gtgatgactctatccctcctgtcagacaac

SEQ ID NO: 72 - DNA, 30 bp, Sus scrofa
gttgtctgacaggagggatagagtcatcac

SEQ ID NO: 73 - DNA, 30 bp, Sus scrofa
ggaagaagtcttaatggaacaagctggagg

SEQ ID NO: 74 - DNA, 43 bp, Sus scrofa
gtcttaagggaacaagctggacgtgatgctactgaaaattttg

SEQ ID NO: 75 - DNA, 32 bp, Sus scrofa
ggtgatgctactgaaagttttgaggatgttgg

SEQ ID NO: 76 - DNA, 30 bp, Sus scrofa
gatgttggacactcctcagatgctcgagag

SEQ ID NO: 77 - DNA, 43 bp, Sus scrofa
gtcttaatggaacaagctggacgtgatgctactgaaagttttg

SEQ ID NO: 78 - DNA, 30 bp, Sus scrofa
gatccagaagcacaagaacagcaagagcac

SEQ ID NO: 79 - DNA, 26 bp, Sus scrofa
caagagcacctgggtaatcctgcacc

SEQ ID NO: 80 - DNA, 37 bp, Sus scrofa
ccatgagtgataacactgcggccaacttacttctgac

(19) United States
(12) Patent Application Publication
(10) Pub. No.: US 2013/0276157 A1
(43) Pub. Date: Oct. 17, 2013

(54) CYB5 AND CYP17 MUTATIONS FOR ALTERATION OF 16-ANDROSTENE STEROID SYNTHESIS AND REDUCED BOAR TAINT IN PIGS

(71) Applicants: Genus, PLC, DeForest, WI (US); E. J. SQUIRES, LTD., Guelph (CA)

(72) Inventor: E. James Squires, Guelph (CA)

(21) Appl. No.: 13/790,678

(22) Filed: Mar. 8, 2013

Related U.S. Application Data
(60) Provisional application No. 61/614,739, filed on Mar. 23, 2012.

(51) Int. Cl. C12N 15/85

(52) U.S. Cl. CPC C12N 15/8509
USPC 800/17; 435/189; 530/401; 514/44 R; 435/6.11; 536/23.5; 536/23.2

(57) ABSTRACT

Novel mutations in cytochrome P450C17 (CYP17) and cytochrome b5 (CYB5) affecting 16-androstene steroid synthesis are disclosed. The novel mutations result in alterations in production of critical intermediaries in the synthesis of 16-androstene steroids. Altering the activity of these enzymes may be useful in enhancing reducing androstenone synthesis and reducing boar taint. The identification of these novel mutations also allows for the development of transgenic pigs bearing mutations in these enzymes or for genetic screening to identify pigs on the basis of their CYP17 and/or CYB5 genotype. Pigs having these mutations may be selected and bred to produce pigs that have a lower incidence of boar taint.

### CROSS-REFERENCE TO RELATED APPLICATIONS

This application claims priority under 35 U.S.C. §119 to provisional application Ser. No. 61/614,739 filed Mar. 23, 2012, herein incorporated by reference in its entirety.

### FIELD OF THE INVENTION

This invention relates generally to modifications in genes and proteins in animals. More particularly, the invention relates to polymorphisms that affect enzyme efficiency and are indicative of heritable phenotypes associated with boar taint in porcine breeds. Methods and compositions for use of these genetic differences in making transgenic animals and for genotyping of animals and selection are also disclosed as well as novel sequences.

### BACKGROUND OF THE INVENTION

Boar taint, an unpleasant odor and flavor emanating from meat originating from intact male pigs, is caused primarily by the accumulation of androstenone and skatole in the fat. Currently, to address the issue of boar taint, boars not selected for breeding are surgically castrated within the first couple of weeks after birth. Castration without any anesthesia induces considerable pain such that stress response and animal welfare are also a concern. Certain countries in Europe, such as the Netherlands and Switzerland, have banned castration without anesthesia and other countries, such as Australia and New Zealand, have moved toward immunocastration. Norway has banned the physical castration of piglets as of 2009 and the Netherlands will follow suit in 2015. Recently, the largest grocer in Belgium, the Colruyt group, announced that it would stop selling pork meat from castrated males by the end of 2010. Thus, high levels of boar taint from intact males are an increasing concern which is emerging in international markets and it is only a matter of time before we can expect this in North American domestic markets.

Androstenone is a 16-androstene steroid produced in the testis as the boar nears puberty, and it acts as a sex pheromone to regulate reproductive development in gilts and induce a mating stance in sows. The pivotal direct cleavage step in the biosynthesis of 16-androstene steroids from progestogens is catalyzed by cytochrome P450C17 (CYP17). This enzyme also catalyzes the 17α-hydroxylation of progestogens and the subsequent C17,20 lyase reaction leading to the biosynthesis of androgens (Lee-Robichaud et al., 2004), a process unrelated to boar taint but vital for the superior growth performance of intact boars. The other components of the cytochrome P450 system, P450 oxidoreductase (POR), cytochrome b5 (CYB5) and cytochrome b5 reductase (CYB5R3), affect which of the three reactions catalyzed by CYP17 predominates. An increased level of POR stimulates the lyase activity to increase androgen production. CYB5 interacts allosterically with the CYP17-POR complex to stimulate the lyase activity as well as the synthesis of 16-androstene steroids. However, the lyase reaction is less dependent on CYB5, while the synthesis of the 16-androstene steroids requires CYB5. We have identified a rare polymorphism in the porcine CYB5 gene just upstream of the translational start site that results in decreased production of CYB5 and decreased synthesis of androstenone (Peacock et al., 2008). CYB5 also interacts with a number of other CYP450 isoforms; a CYB5 knockout mouse model has a dramatically altered expression of drug metabolizing enzymes and low levels of testicular androgens (McLaughlin et al., 2010). Therefore, totally eliminating the expression of CYB5 would result in decreased synthesis of androgens as well as decreased synthesis of androstenone.

In view of the foregoing, further work is needed to fully understand androstenone synthesis and the production of androgens. Understanding the biochemical events involved in androstenone synthesis and the production of androgens can lead to novel strategies for treating, reducing or preventing boar taint. In addition, polymorphisms in these candidate genes may be useful as possible markers for low boar taint pigs.

### SUMMARY OF THE INVENTION

This invention relates to the development and creation of mutations in cytochrome b5 (CYB5) and cytochrome P450c17 (CYP17) that alter the production of steroids involved in the synthesis of 16-androstene steroids. CYB5 stimulates the formation of 16-androstene steroids by CYP17 leading to androstenone synthesis, as well as the lyase reaction leading to the production of androgens. Applicants have identified several target mutation sites in CYB5A that will decrease the stimulatory effect of CYB5A on the synthesis of the 16-androstene steroids, while maintaining the normal production of sex steroids. Applicants mutated those residues in CYB5A that are involved in binding to the CYP17-POR complex, taking initial direction from the sequence of CYB5B, which does not stimulate 16-androstene synthesis but does increase the lyase reaction. Since the formation of 16-androstene steroids is more sensitive to CYB5 than the lyase reaction, the development of this less functional form of CYB5 decreases 16-androstene steroid synthesis while maintaining the normal production of sex steroids. Applicants have also identified and mutated those residues in porcine CYP17 that are responsible for the synthesis of the 16-androstene steroids. Since rat CYP17 does not catalyse the formation of 16-androstenes as effectively as porcine CYP17, a comparison of the sequences of CYP17 between pig, rat and human identified potential amino acid targets to mutate. Applicants have identified several sites, based upon study and comparison of sequences from these different species, which are involved and critical for activity of CYB5 and/or CYP17 in the 16-androstene steroid pathway. To the extent that this family of genes are conserved among species and animals, it is expected that the different alleles disclosed herein will also correlate with variability in these gene(s) in other economic or meat-producing animals such as cattle, sheep, chicken, etc. with concomitant effects on enzyme activity related to other traits in lieu of or in addition to boar taint.

To achieve the objectives and in accordance with the purpose of the invention, as embodied and broadly described herein, the present invention provides a method for altering the activity of CYP17 and/or CYB5, and thereby reducing boar taint comprising decreasing 16-androstene synthesis in a pig.

The activity of CYP17 can be altered by modification of the amino acid present at one or more positions selected from the group consisting of: amino acids 102, 103, 104, 106, 108, 109, 112, 202, 344, 345, 348, 352 and 454 of the porcine CYP17 protein (SEQ ID NO:4). In a more preferred embodiment the modification comprises a glutamine at amino acid 102 (SEQ ID NO:32), a serine at amino acid 103 (SEQ ID NO:33), a leucine at amino acid 104 (SEQ ID NO:34), an alanine at amino acid 106 (SEQ ID NO:35), an aspartic acid at amino acid 106 (SEQ ID NO:36), a glutamine at amino acid 108 (SEQ ID NO:37), a glycine at amino acid 109 (SEQ ID NO:37), a valine at amino acid 112 (SEQ ID NO:38), a threonine at amino acid 202 (SEQ ID NO:39), a phenylalanine at amino acid 344 (SEQ ID NO:40), an asparagine at amino acid 345 (SEQ ID NO:40), a serine at amino acid 348 (SEQ ID NO:41), a methionine at amino acid 352 (SEQ ID NO:42), and/or a valine at amino acid 454 (SEQ ID NO:43).

The activity of CYB5 can be altered by modification of the amino acid at one or more positions selected from the group consisting of: amino acids 21, 28, 52, 57, 62 and/or 70 of the porcine CYB5A protein (SEQ ID NO:2). In a more preferred embodiment, the modification comprises a methionine at position 52 (SEQ ID NO:25), an arginine at position 57 (SEQ ID NO:26), a serine at position 62 (SEQ ID NO:27), a serine at position 70 (SEQ ID NO:28), a lysine at position 21 (SEQ ID NO:23), and/or a valine at position 28 (SEQ ID NO:24).

The present invention also provides novel CYP17 proteins that modify 16-androstene steroid activity or production in pigs. The CYP17 proteins comprise alterations in the amino acid sequence that may include alterations at amino acid 102, 103, 104, 106, 108, 109, 112, 202, 344, 345, 348, 352 and/or 454. In a more preferred embodiment the modification comprises a glutamine at amino acid 102 (SEQ ID NO:32), a serine at amino acid 103 (SEQ ID NO:33), a leucine at amino acid 104 (SEQ ID NO:34), an alanine at amino acid 106 (SEQ ID NO:35), an aspartic acid at amino acid 106 (SEQ ID NO:36), a glutamine at amino acid 108 (SEQ ID NO:37), a glycine at amino acid 109 (SEQ ID NO:37), a valine at amino acid 112 (SEQ ID NO:38), a threonine at amino acid 202 (SEQ ID NO:39), a phenylalanine at amino acid 344 (SEQ ID NO:40), an asparagine at amino acid 345 (SEQ ID NO:40), a serine at amino acid 348 (SEQ ID NO:41), a methionine at amino acid 352 (SEQ ID NO:42), and/or a valine at amino acid 454 (SEQ ID NO:43).

The present invention also provides novel CYB5 proteins that modify 16-androstene steroid activity or production in pigs. The CYB5 proteins comprise alterations in the amino acid sequence that may include alterations at amino acid 21, 28, 52, 57, 62 and/or 70. In a more preferred embodiment, the modification comprises a methionine at position 52 (SEQ ID NO:25), an arginine at position 57 (SEQ ID NO:26), a serine at position 62 (SEQ ID NO:27), a serine at position 70 (SEQ ID NO:28), a lysine at position 21 (SEQ ID NO:23), and/or a valine at position 28 (SEQ ID NO:24).

The present invention also provides transgenic animals with altered 16-androstene steroid synthesis and concomitant characteristics. The transgenic animal may comprise a modified CBY5 and/or CYP17 protein as described above, or produced by the methods above.

The present invention also provides the polynucleotides encoding the modified CYB5 and CYP17 proteins, and which may be used in the methods described for altering 16-androstene steroid synthesis. In a preferred embodiment, the polynucleotides of the present invention have at least 90% sequence identity over the entire sequence to sequences provided herein, and specifically SEQ ID NOS:23-50.

In addition, the present invention provides the discovery of alternate genotypes and gene mutations that provide compositions and methods for reducing 16-androstene synthesis. The mutations of the present invention may also provide a method for genetically typing animals and screening animals for reduced 16-androstene synthesis and reduced boar taint.

The accompanying Figures, which are incorporated herein and which constitute a part of this specification, illustrates one embodiment of the invention and, together with the description, serve to explain the principles of the invention.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 shows the steroid binding pocket of CYP17A1. Human CYP17A1 (SEQ ID NO:5); Pig CYP17A1 (SEQ ID NO:6); Rat CYP17A1 (SEQ ID NO:7). Positions are indicated in relation to SEQ ID NO:4.

FIG. 2 shows the regions involved in binding of CYB5 to CYP17. (A) The region of CYP17 between amino acids 340 and 369 is shown, with the arginine at position 347 and 358 in bold. Human CYP17A1 (SEQ ID NO:8); Pig CYP17A1 (SEQ ID NO:9); Rat CYP17A1 (SEQ ID NO:10). Positions are indicated in relation to SEQ ID NO:4. (B) The region of CYP17 between amino acids 440 and 469 is shown, with the arginine at position 449 in bold. Human CYP17A1 (SEQ ID NO:11); Pig CYP17A1 (SEQ ID NO:12); Rat CYP17A1 (SEQ ID NO:13). Positions are indicated in relation to SEQ ID NO:4.

FIG. 3 shows a comparison of the amino acid sequences of CYB5A (SEQ ID NO:14) and CYB5B (SEQ ID NO:15) from position 43 to 74.

FIG. 4 shows a comparison of the sequence of CYB5A between rat, human and pig from amino acid 1 to 50. Rat CYB5 (SEQ ID NO:16); Human CYB5 (SEQ ID NO:17); Pig CYB5 (SEQ ID NO:18). Positions are indicated in relation to SEQ ID NO:2.

FIGS. 5 (A and B) shows a sequence alignment of CYP17A1 from pig, human (hum) and rat. Pig CYP17 (SEQ ID NO:4); Human CYP17A1 (SEQ ID NO:19); Rat CYP17A1 (SEQ ID NO:20).

FIG. 6 shows a sequence alignment of rat, human (hum) and pig CYB5A. Pig CYB5A (SEQ ID NO:2); Human CYB5A (SEQ ID NO:21); Rat CYB5A (SEQ ID NO:22).

FIG. 7 shows the nucleotide sequence of pig CYB5 (SEQ ID NO:1).

FIG. 8 shows the amino acid sequence of pig CYB5 (SEQ ID NO:2).

FIG. 9 shows the nucleotide sequence of pig CYP17 (SEQ ID NO:3).

FIG. 10 shows the amino acid sequence of pig CYP17 (SEQ ID NO:4).

FIG. 11(A-O) shows the nucleotide sequences encoding the various altered CYB5 and CYP17 proteins. CYB5A-N21K (SEQ ID NO:23); CYB5A-L28V (SEQ ID NO:24); CYB5A-R52M (SEQ ID NO:25); CYB5A-G57R (SEQ ID NO:26); CYB5A-N62S (SEQ ID NO:27); CYB5A-T70S (SEQ ID NO:28); CYB5A-N21K+L28V (SEQ ID NO:29); CYB5A-R52M+N62S (SEQ ID NO:30); CYB5A-QM R52M/G57R/N62S/T70S (SEQ ID NO:31); CYP17A1-L102Q (SEQ ID NO:32); CYP17A1-D103S (SEQ ID NO:33); CYP17A1-I104L (SEQ ID NO:34); CYP17A1-S106A (SEQ ID NO:35); CYP17A1-S106D (SEQ ID NO:36); CYP17A1-NQ108QG (SEQ ID NO:37); CYP17A1-I112V (SEQ ID NO:38); CYP17A1-N202T (SEQ ID NO:39); CYP17A1-IS344FN (SEQ ID NO:40); CYP17A1-N348S (SEQ ID NO:41); CYP17A1-L352M (SEQ ID NO:42); CYP17A1-L454V (SEQ ID NO:43); CYP17A1-L102Q+112V (DM) (SEQ ID NO:44); CYP17A1-L102Q+D103S+I112V (TM) (SEQ ID NO:45); CYP17A1-SM L102Q+D103S+I104L+NQ108QG+I112V (SEQ ID NO:46); CYP17A1 D103S+L454V (SEQ ID NO:47); CYP17A1 D103S+I104L+L454V (IDL) (SEQ ID NO:48); CYP17A1 D103S+S106A+L454V (SDL) (SEQ ID NO:49); CYP17A1-(STM) S106A+L102Q+D102S+L112V (SEQ ID NO:50).

### DETAILED DESCRIPTION OF THE INVENTION

Reference will now be made in detail to the presently referred embodiments of the invention, which together with the following examples, serve to explain the principles of the invention.

The invention relates to mutations in CYP17 and CYB5, and methods of expressing altered CYP17 and CYB5 in an animal of a particular breed, strain, population, or group, whereby the animal is more likely to have reduced 16-androstene steroid synthesis and yield desired boar taint traits.

### CYP17

Phosphorylation of serine and threonine residues of human CYP17 increases the lyase activity with no effect on the 17α-hydroxylation reaction (Pandley and Miller, 2005). Our own unpublished work has shown that altering the phosphorylation status of porcine CYP17 in the presence of CYB5 can increase the lyase activity and at the same time decrease the synthesis of 16-androstene steroids. Thus, altering the phosphorylation status of porcine CYP17 is a potential method for decreasing the synthesis of androstenone while maintaining the synthesis of androgens and estrogens. However, while many potential serine/threonine phosphorylation sites have been investigated in human CYP17 (Wang et al., 2010), the site of phosphorylation that affects the lyase activity has not yet been identified.

A model of human CYP17 has been generated (Auchus and Miller, 1999) and used to predict the amino acid residues that are involved in the 17α-hydroxylation and lyase reactions. Mutation of either Arg347, Arg 358 or Arg 449 to alanine in human CYP17 prevented binding of CYB5 and eliminated the C17,20 lyase activity and the formation of the 16-androstene steroids, with no effect on the hydroxylase activity (Lee-Robichaud et al., 2004). The effects of these mutations in porcine CYP17 on the synthesis of 16-androstene steroids have not been reported.

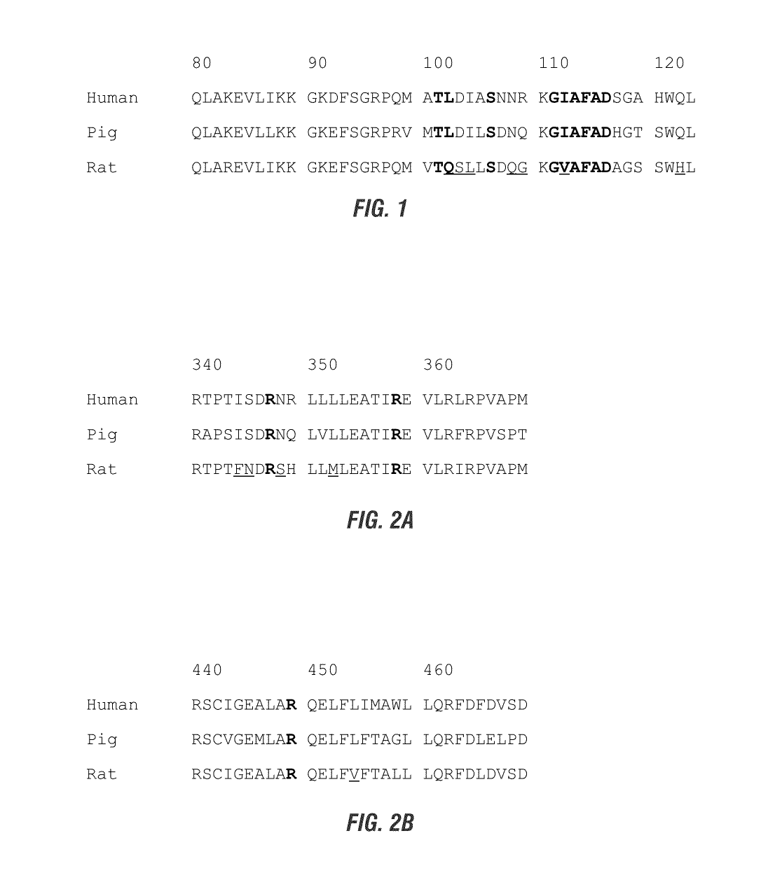

The steroid binding pocket of CYP17 includes two regions, amino acids 101-102 and 111-116 (shown in bold in FIG. 1).

There have been no reported mutations in CYP17 that differentially affect the C17,20 lyase reaction from the formation of the 16-androstenes. However, rat testis does not form 16-androstenes (Cooke and Gower, 1977) and rat CYP17 differs by having glutamine at position 102 and valine at position 112 compared to leucine at position 102 and isoleucine at position 112 in porcine and human CYP17, which both catalyse the formation of 16-androstenes. This suggests that mutation of leucine 102 to glutamine and isoleucine 112 to valine might decrease the formation of 16-androstenes by porcine CYP17 while not adversely affecting the lyase and hydroxylase activities. Other residues in this region may also be good candidates, especially those that are near the critical residues 102 and 112, such as residues 103 and 109 that are different in rat compared to human and porcine CYP17. Swart et al (2010) have shown that mutation of leucine 105 in pig CYP17 to alanine (as is found in human CYP17) dramatically decreases the C17,20 lyase activity, increases the 17α-hydroxylase activity and eliminates the stimulatory effect of CYB5. The conserved serine 106 is required for both the lyase and hydroxylase activities.

### CYB5

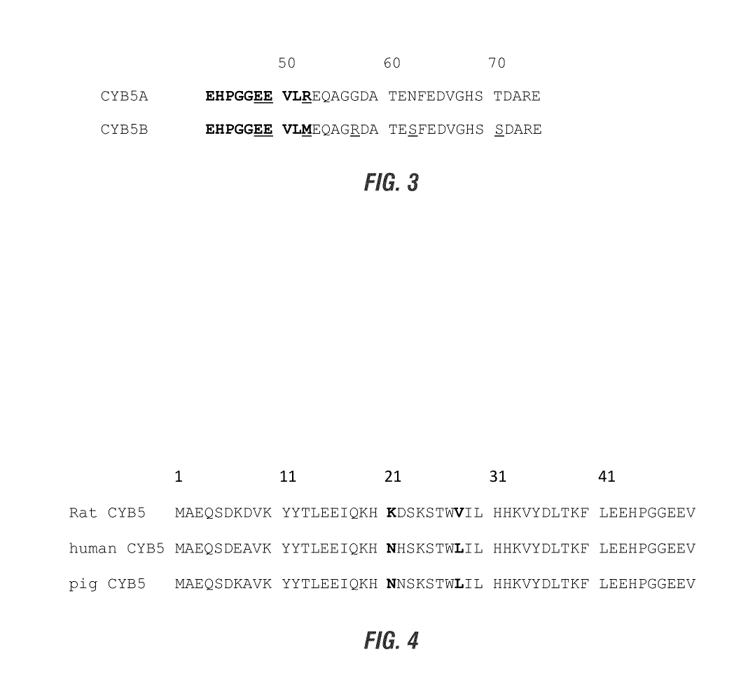

There are two forms of CYB5, the microsomal CYB5A that is involved in regulating steroidogenesis and the so-called ‘outer mitochondrial’ CYB5B. We have shown that while CYB5A stimulates both the lyase activity and formation of the 16-androstene steroids by porcine CYP17, CYB5B only stimulates the lyase reaction but has no effect on the synthesis of 16-androstene steroids (Billen and Squires, 2009). Mutational studies with CYB5 have identified the region on the surface of human CYB5 that is involved in binding of human CYB5A to the CYP17-POR complex. Mutation of amino acid residues E48, E49 and R52 to glycine reduced the stimulation of C17,20 lyase activity by CYB5A but did not affect the capacity of the protein to bind heme or accept electrons from POR (Naffin-Olivod and Auchus, 2006). Comparing the region between CYB5A and CYB5B (FIG. 3), it can be seen that while E48 and E49 are conserved, R52 is a methionine in CYB5B, while the rest of this region is almost completely conserved. This suggests that mutation of R52 in CYB5A to methionine would shift the activity of CYB5A to be more like CYB5B. Other amino acid residues in this region might also be good candidates for mutation, such as residues 57, 62 and 69 which differ between CYB5A and CYB5B. This would dramatically reduce the synthesis of the 16-androstene steroids while maintaining the normal production of androgens and estrogens. Other amino acids in this region, including D58, D65 and E74 are also required for effective stimulation of lyase activity (Naffin-Olivod and Auchus, 2006).

The present invention provides a method for altering the activity of CYP17 and/or CYB5, and thereby reducing boar taint comprising decreasing 16-androstene synthesis in a pig.

The activity of CYP17 can be altered by modification of the amino acid present at one or more positions selected from the group consisting of: amino acids 102, 103, 104, 106, 108, 109, 112, 202, 344, 345, 348, 352 and 454 of the porcine CYP17 protein. In a more preferred embodiment the modification comprises a glutamine at amino acid 102, a serine at amino acid 103, a leucine at amino acid 104, an alanine at amino acid 106, an aspartic acid at amino acid 106, a glutamine at amino acid 108, a glycine at amino acid 109, a valine at amino acid 112, a threonine at amino acid 202, a phenylalanine at amino acid 344, an asparagine at amino acid 345, a serine at amino acid 348, a methionine at amino acid 352, and/or a valine at amino acid 454.

The activity of CYB5 can be altered by modification of the amino acid at one or more positions selected from the group consisting of: amino acids 21, 28, 52, 57, 62 and/or 70 of the porcine CYB5A protein. In a more preferred embodiment, the modification comprises a methionine at position 52, an arginine at position 57, a serine at position 62, a serine at position 70, a lysine at position 21, and/or a valine at position 28.

The present invention also provides novel CYP17 proteins that modify 16-androstene steroid activity or production in pigs. The CYP17 proteins comprise alterations in the amino acid sequence that may include alterations at amino acid 102, 103, 104, 106, 108, 109, 112, 202, 344, 345, 348, 352 and/or 454. In a more preferred embodiment, the In a more preferred embodiment the modification comprises a glutamine at amino acid 102 (SEQ ID NO:32), a serine at amino acid 103 (SEQ ID NO:33), a leucine at amino acid 104 (SEQ ID NO:34), an alanine at amino acid 106 (SEQ ID NO:35), an aspartic acid at amino acid 106 (SEQ ID NO:36), a glutamine at amino acid 108 (SEQ ID NO:37), a glycine at amino acid 109 (SEQ ID NO:37), a valine at amino acid 112 (SEQ ID NO:38), a threonine at amino acid 202 (SEQ ID NO:39), a phenylalanine at amino acid 344 (SEQ ID NO:40), an asparagine at amino acid 345 (SEQ ID NO:40), a serine at amino acid 348 (SEQ ID NO:41), a methionine at amino acid 352 (SEQ ID NO:42), and/or a valine at amino acid 454 (SEQ ID NO:43).

The present invention also provides novel CYB5 proteins that modify 16-androstene steroid activity or production in pigs. The CYB5 proteins comprise alterations in the amino acid sequence that may include alterations at amino acid 21, 28, 52, 57, 62 and/or 70. In a more preferred embodiment, the modification comprises a methionine at position 52 (SEQ ID NO:25), an arginine at position 57 (SEQ ID NO:26), a serine at position 62 (SEQ ID NO:27), a serine at position 70 (SEQ ID NO:28), a lysine at position 21 (SEQ ID NO:23), and/or a valine at position 28 (SEQ ID NO:24).

The present invention also provides transgenic animals with altered 16-androstene steroid synthesis and concomitant characteristics. The transgenic animal may comprise a modified CBY5 and/or CYP17 protein as described above, or produced by the methods above.

The present invention also provides the polynucleotides encoding the modified CYB5 and CYP17 proteins, and which may be used in the methods described for altering 16-androstene steroid synthesis. In a preferred embodiment, the polynucleotides of the present invention have at least 90% sequence identity over the entire sequence to SEQ ID NOS:23-50.

In addition, the present invention provides the discovery of alternate genotypes and gene mutations that provide compositions and methods for reducing 16-androstene synthesis. The mutations of the present invention may also provide a method for genetically typing animals and screening animals for reduced 16-androstene synthesis and reduced boar taint.

The following is a general overview of techniques which can be used for the methods and compositions of the invention and to assay for the genetic marker of the invention.

In the present invention, a sample of genetic material is obtained from an animal. Samples can be obtained from blood, tissue, semen, etc. Generally, peripheral blood cells are used as the source, and the genetic material is DNA. A sufficient amount of cells are obtained to provide a sufficient amount of DNA for analysis. This amount will be known or readily determinable by those skilled in the art. The DNA is isolated from the blood cells by techniques known to those skilled in the art.

### Isolation and Amplification of Nucleic Acid

Samples of genomic DNA are isolated from any convenient source including saliva, buccal cells, hair roots, blood, cord blood, amniotic fluid, interstitial fluid, peritoneal fluid, chorionic villus, and any other suitable cell or tissue sample with intact interphase nuclei or metaphase cells. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W.H. Freeman & Co. New York (1992). Genomic DNA can also be isolated from cultured primary or secondary cell cultures or from transformed cell lines derived from any of the aforementioned tissue samples.

Samples of animal RNA can also be used. RNA can be isolated from tissues expressing the gene as described in Sambrook et al., supra. RNA can be total cellular RNA, mRNA, poly A+ RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al., Hum. Genet. 85:655-658 (1990).

### PCR Amplification

The most common means for amplification is polymerase chain reaction (PCR), as described in U.S. Pat. Nos. 4,683,195; 4,683,202; and 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on a sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl) in a buffer of 50 mM Tris-HCl (pH 8.3), 50 mM KCl 1.5 mM MgCl₂, 0.5% Tween 20, and 0.5% NP40 supplemented with 100 μg/ml of proteinase K. After incubating at 56° C. for 2 hr. the cells are heated to 95° C. for 10 min to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs. at 50°-60° C. and then at 95° C. for 10 minutes to inactivate the proteinase. During longer incubations, fresh proteinase K should be added after about 4 hrs. at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 μl of PCR lysis buffer (10 mM Tris-HCl (pH 8.3), 50 mM KCl, 2.5 mM MgCl₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N. H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

### Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:12427-2448 (1989).

### Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed so that under low stringency, they will bind to both polymorphic forms of the allele, but at high stringency, bind to the allele to which they correspond. Alternatively, stringency conditions can be devised in which an essentially binary response is obtained, i.e., an ASO corresponding to a variant form of the target gene will hybridize to that allele, and not to the wild-type allele.

### Ligase Mediated Allele Detection Method

Target regions of a test subject's DNA can be compared with target regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

### Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (Tₘ). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, “Principles and Applications for DNA Amplification”, W.H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tₘ's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining

### Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

### Single-Strand Conformation Polymorphism Analysis

Target sequences or alleles at the chosen boar taint loci can be differentiated using single-strand conformation polymorphism analysis, which identifies base differences by alteration in electrophoretic migration of single-stranded PCR products, as described in Orita et al., Proc. Nat. Acad. Sci. 85:2766-2770 (1989). Amplified PCR products can be generated as described above, and heated or otherwise denatured, to form single-stranded amplification products. Single-stranded nucleic acids may refold or form secondary structures which are partially dependent on the base sequence. Thus, electrophoretic mobility of single-stranded amplification products can detect base-sequence difference between alleles or target sequences.

### Chemical or Enzymatic Cleavage of Mismatches

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from an animal and an affected family member may be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms that may be associated with polymorphisms.

### Non-Gel Systems

Other possible techniques include non-gel systems such as TAQMAN™ (Perkin Elmer). In this system, oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e., there is a mismatch of some form, the cleavage of the dye does not take place. Thus, only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Yet another technique includes an Invader Assay, which includes isothermic amplification that relies on a catalytic release of fluorescence. See Third Wave Technology at www.twt.com.

### Non-PCR Based DNA Diagnostics

The identification of a DNA sequence linked to sequences encoding CYB5 and/or CYP17 can be made without an amplification step, based on polymorphisms including restriction fragment length polymorphisms in an animal and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with P³² or S³⁵. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to the porcine chromosome where the CYB5 or CYP17 genes reside, and thus defining a genetic marker linked to the gene, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

According to the invention, polymorphisms in genes encoding CYP17 and CYB5 have been identified which alter the synthesis of 16-androstene steroids and have an association with boar taint. The presence or absence of the markers, in one embodiment may be assayed by PCR-RFLP analysis using the restriction endonucleases and amplification primers may be designed using analogous human, pig or other sequences due to the high homology in the region surrounding the polymorphisms, or may be designed using known gene sequence data as exemplified in GenBank or even designed from sequences obtained from linkage data from closely surrounding genes based upon the teachings and references herein. The sequences surrounding the polymorphism will facilitate the development of alternate PCR tests in which a primer of about 4-30 contiguous bases taken from the sequence immediately adjacent to the polymorphism is used in connection with a polymerase chain reaction to greatly amplify the region before treatment with the desired restriction enzyme. The primers need not be the exact complement; substantially equivalent sequences are acceptable. The design of primers for amplification by PCR is known to those of skill in the art and is discussed in detail in Ausubel (ed.), Short Protocols in Molecular Biology, 4th Edition, John Wiley and Sons (1999).

The following is a brief description of primer design.

### Primer Design Strategy

Increased use of polymerase chain reaction (PCR) methods has stimulated the development of many programs to aid in the design or selection of oligonucleotides used as primers for PCR. Four examples of such programs that are freely available via the Internet are: PRIMER by Mark Daly and Steve Lincoln of the Whitehead Institute (UNIX, VMS, DOS, and Macintosh), Oligonucleotide Selection Program (OSP) by Phil Green and LaDeana Hiller of Washington University in St. Louis (UNIX, VMS, DOS, and Macintosh), PGEN by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

### Sequencing and PCR Primers

Designing oligonucleotides for use as either sequencing or PCR primers requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding program such as those described above. If a possible stem structure is observed, the sequence of the primer can be shifted a few nucleotides in either direction to minimize the predicted secondary structure. The sequence of the oligonucleotide should also be compared with the sequences of both strands of the appropriate vector and insert DNA. Obviously, a sequencing primer should only have a single match to the target DNA. It is also advisable to exclude primers that have only a single mismatch with an undesired target DNA sequence. For PCR primers used to amplify genomic DNA, the primer sequence should be compared to the sequences in the GenBank database to determine if any significant matches occur. If the oligonucleotide sequence is present in any known DNA sequence or, more importantly, in any known repetitive elements, the primer sequence should be changed.

The methods and materials of the invention may also be used more generally to evaluate pig DNA, genetically type individual pigs, and detect genetic differences in pigs. In particular, a sample of pig genomic DNA may be evaluated by reference to one or more controls to determine if a polymorphism in the particular gene is present. Preferably, RFLP analysis is performed with respect to the pig gene, and the results are compared with a control. The control is the result of a RFLP analysis of the pig gene of a different pig where the polymorphism(s) of the pig gene is/are known. Similarly, the genotype of a pig may be determined by obtaining a sample of its genomic DNA, conducting RFLP analysis of the gene in the DNA, and comparing the results with a control. Again, the control is the result of RFLP analysis of the gene of a different pig. The results genetically type the pig by specifying the polymorphism(s) in its genes. Finally, genetic differences among pigs can be detected by obtaining samples of the genomic DNA from at least two pigs, identifying the presence or absence of a polymorphism in the gene, and comparing the results.

These assays are useful for identifying the genetic markers relating to boar taint, as discussed above, for identifying other polymorphisms in the genes encoding enzymes involved in 16-androstene synthesis and for the general scientific analysis of pig genotypes and phenotypes.

The examples and methods herein disclose certain gene(s) which has been identified to have a polymorphism(s) which is associated either positively or negatively with a beneficial trait that will have an effect on boar taint for animals carrying this polymorphism. The identification of the existence of a polymorphism within a gene is often made by a single base alternative that results in a restriction site in certain allelic forms. A certain allele, however, as demonstrated and discussed herein, may have a number of base changes associated with it that could be assayed for which are indicative of the same polymorphism (allele). Further, other genetic markers or genes may be linked to the polymorphisms disclosed herein so that assays may involve identification of other genes or gene fragments, but which ultimately rely upon genetic characterization of animals for the same polymorphism. Any assay which sorts and identifies animals based upon the allelic differences disclosed herein are intended to be included within the scope of this invention.

As used herein a “favorable” or “desired” or “improved” with respect to a trait means a significant improvement (increase or decrease) in one of any measurable indicia of boar taint or other related phenotype above the mean of a given group, species line or population, so that this information can be used in breeding to achieve a uniform population which is optimized for these traits. This may include an increase in some traits or a decrease in others depending on the desired characteristics. Traits may also be observed at the molecular level by assaying for activity of enzymes involved in 16-androstene synthesis.

Methods for assaying for these traits generally comprises the steps 1) obtaining a biological sample from an animal; and 2) analyzing the genomic DNA or protein obtained in 1) to determine which allele(s) is/are present. Haplotype data which allows for a series of linked polymorphisms to be combined in a selection or identification protocol to maximize the benefits of each of these markers may also be used.

Since several of the polymorphisms may involve changes in amino acid composition of the respective protein or will be indicative of the presence of this change, assay methods may even involve ascertaining the amino acid composition of the protein of the major effect genes of the invention. Methods for this type or purification and analysis typically involve isolation of the protein through means including fluorescence tagging with antibodies, separation and purification of the protein (i.e. through reverse phase HPLC system), and use of an automated protein sequencer to identify the amino acid sequence present. Protocols for this assay are standard and known in the art and are disclosed in Ausubel et. al. (eds.), Short Protocols in Molecular Biology Fourth ed. John Wiley and Sons 1999.

One of skill will readily understand that the modified CYB5 and CYP17 protein sequences also describe all of the corresponding RNA and DNA sequences which encode the polypeptides, by conversion of the amino acid sequence into the corresponding nucleotide sequence using the genetic code, by alternately assigning each possible codon in each possible codon position. Similarly, each nucleic acid sequence which is provided also inherently provides all of the nucleic acids which encode the same protein, since one of skill simply translates a selected nucleic acid into a protein and then uses the genetic code to reverse translate all possible nucleic acids from the amino acid sequence.

The sequences also provide a variety of conservatively modified variations by substituting appropriate residues with the exemplar conservative amino acid substitutions provided

In another embodiment, the invention comprises a method for identifying genetic markers for boar taint. Once a major effect gene has been identified, it is expected that other variation present in the same gene, allele or in related family of gene sequences in useful linkage disequilibrium therewith may be used to identify similar effects on these traits. The identification of other such genetic variation, once a major effect gene has been discovered, represents more than routine screening and optimization of parameters well known to those of skill in the art and is intended to be within the scope of this invention.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison to in this case the Reference sequences. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e)(I) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

These programs and algorithms can ascertain the analogy of a particular polymorphism in a target gene to those disclosed herein. It is expected that this polymorphism will exist in other animals and use of the same in other animals than disclosed herein involved no more than routine optimization of parameters using the teachings herein.

It is also possible to establish linkage between specific alleles of alternative DNA markers and alleles of DNA markers known to be associated with a particular gene (e.g. the genes discussed herein), which have previously been shown to be associated with a particular trait. Thus, in the present situation, taking one or both of the genes, it would be possible, at least in the short term, to select for animals likely to produce desired traits, or alternatively against animals likely to produce less desirable traits indirectly, by selecting for certain alleles of an associated marker through the selection of specific alleles of alternative chromosome markers. As used herein the term “genetic marker” shall include not only the nucleotide polymorphisms disclosed by any means of assaying for the protein changes associated with the polymorphism, be they linked markers, use of microsatellites, or even other means of assaying for the causative protein changes indicated by the marker and the use of the same to influence traits of an animal.

Another indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein.

In accordance with the present invention, nucleic acids having the appropriate level sequence homology (i.e., 70% identity or greater) with part or all the coding regions of SEQ ID NOS:23-50 may be identified by using hybridization and washing conditions of appropriate stringency. For example, hybridizations may be performed, according to the method of Sambrook et al., using a hybridization solution comprising: 1.0% SDS, up to 50% formamide, 5×SSC (150 mM NaCl, 15 mM trisodium citrate), 0.05% sodium pyrophosphate (pH 7.6), 5×Denhardt's solution, and 100 microgram/ml denatured, sheared salmon sperm DNA. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes to 1 hour at 37° C. in 2×SSC and 0.1% SDS; (4) 2 hours at 45-55° C. in 2×SSC and 0.1% SDS, changing the solution every 30 minutes.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and wash in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and wash in 1×SSC and 0.5% SDS at 6-5° C. for 15 minutes. Very high stringency hybridization is defined as hybridization in 6×SSC, 5×Denhardt's solution, 0.5% SDS and 100 μg/ml denatured salmon sperm DNA at 42° C., and wash in 0.1 SSC and 0.5% SDS at 65° C. for 15 minutes.

Nucleic acids of the present invention may be maintained as DNA in any convenient cloning vector. In a preferred embodiment, clones are maintained in plasmid cloning/expression vector, such as PBLUESCRIPT (STRATAGENE, La Jolla, Calif.), that is propagated in a suitable E. coli host cell.

The polypeptides of the invention may be altered in various ways including amino acid substitutions, deletions, truncations, and insertions. Novel proteins having properties of interest may be created by combining elements and fragments of proteins of the present invention, as well as with other proteins. Methods for such manipulations are generally known in the art. Thus, the genes and nucleotide sequences of the invention include both the naturally occurring sequences as well as mutant forms. Likewise, the proteins of the invention encompass naturally occurring proteins as well as variations and modified forms thereof. Such variants will continue to possess the desired CYB5 and CYP17 activities. Obviously, the mutations that will be made in the DNA encoding the variant must not place the sequence out of reading frame and preferably will not create complementary regions that could produce secondary mRNA structure.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays where the effects of the CYB5 and CYP17 proteins can be observed.

As used herein, often the designation of a particular polymorphism is made by the name of a particular restriction enzyme. This is not intended to imply that the only way that the site can be identified is by the use of that restriction enzyme. There are numerous databases and resources available to those of skill in the art to identify other restriction enzymes which can be used to identify a particular polymorphism, for example http://darwin.bio.geneseo.edu which can give restriction enzymes upon analysis of a sequence and the polymorphism to be identified. In fact as disclosed in the teachings herein there are numerous ways of identifying a particular polymorphism or allele with alternate methods which may not even include a restriction enzyme, but which assay for the same genetic or proteomic alternative form.

As used herein, the term “express” or “expression” is defined to mean transcription and translation. The regulatory elements are operably linked to the coding sequence of the CYB5 and CYP17 genes such that the regulatory element is capable of controlling expression of the CYB5 and CYP17 genes. “Altered levels” or “altered expression” refers to the production of gene product(s) in transgenic organisms in amounts or proportions that differ from that of normal or non-transformed organisms.

“RNA transcript” refers to the product resulting from RNA polymerase-catalyzed transcription of a DNA sequence. When the RNA transcript is a perfect complimentary copy of the DNA sequence, it is referred to as the primary transcript or it may be an RNA sequence derived from posttranscriptional processing of the primary transcript and is referred to as the mature RNA. “Messenger RNA (mRNA)” refers to the RNA that is without introns and that can be translated into polypeptides by the cell. “cDNA” refers to a DNA that is complementary to and derived from an mRNA template. The cDNA can be single-stranded or converted to double stranded form using, for example, the Klenow fragment of DNA polymerase I. “Sense” RNA refers to an RNA transcript that includes the mRNA and so can be translated into a polypeptide by the cell. “Antisense”, when used in the context of a particular nucleotide sequence, refers to the complementary strand of the reference transcription product. “Antisense RNA” refers to an RNA transcript that is complementary to all or part of a target primary transcript or mRNA and that blocks the expression of a target gene. The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

As used herein, the terms “encoding”, “coding”, or “encoded” when used in the context of a specified nucleic acid mean that the nucleic acid comprises the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

A “protein” or “polypeptide” is a chain of amino acids arranged in a specific order determined by the coding sequence in a polynucleotide encoding the polypeptide. Each protein or polypeptide has a unique function.

One of skill in the art, once a polymorphism has been identified and a correlation to a particular trait established will understand that there are many ways to genotype animals for this polymorphism. The design of such alternative tests merely represents optimization of parameters known to those of skill in the art and is intended to be within the scope of this invention as fully described herein.

The following non-limiting examples are illustrative of the present invention:

### Methodology:

The present invention utilizes two approaches: (1) mutate the residues on CYB5A that are necessary for CYB5 to interact with the CYP17-POR complex and stimulate 16-androstene steroid synthesis; and (2) identify and mutate those residues in porcine CYP17 that are responsible for the synthesis of the 16-androstene steroids. We compared the sequences of CYP17 between pig, human and rat to identify potential targets to mutate. We tested the effect of the mutations in CYB5 and CYP17 in our in vitro system in which we express porcine POR, CYB5, CYB5R3 and CYP17A1 in HEK273 cells (Billen and Squires, 2009) and measured the formation of metabolites from radiolabelled pregnenolone by HPLC. This allowed us to rapidly screen for those amino acid changes that reduce the formation of the 16-androstene steroids but do not affect the 17α-hydroxylase and C17,20 lyase reactions that are necessary for the synthesis of androgens and estrogens. The generation of an appropriate construct allows for transfection into isolated Leydig cells to confirm its function in primary cells before developing a transgenic knock-in pig with the desired mutation.

### EXAMPLES

### Example 1

### CYB5A and CYP17A1 Mutations for Alteration of 16-Androstene Steroid Synthesis and Reduced Boar Taint in Pigs

Cytochrome b5 (CYB5) stimulates the formation of 16-androstene steroids by cytochrome P450c17A1 (CYP17A1) leading to androstenone synthesis, as well as the lyase reaction leading to the production of sex steroids. We have identified several target mutation sites in CYB5A that have potential to decrease the stimulatory effect of CYB5A on the synthesis of the 16-androstene steroids, while maintaining the normal production of sex steroids. This includes those residues in CYB5A that are involved in binding to the CYP17-POR complex, taking initial direction from the sequence of CYB5B, which does not stimulate 16-androstene synthesis but does increase the lyase reaction, and rat CYB5A, since rat testis does not form 16-androstene steroids. Since the formation of 16-androstene steroids is more sensitive to CYB5 than the lyase reaction, the development of this less functional form of CYB5 should decrease 16-androstene steroid synthesis while maintaining the normal production of sex steroids. We have also identified and mutated those residues in porcine CYP17 that are responsible for the synthesis of the 16-androstene steroids. Since rat CYP17 does not catalyse the formation of 16-androstenes as effectively as porcine CYP17, a comparison of the sequences of CYP17 between pig, rat and human identified potential amino acid targets to mutate. We have identified several sites, based upon study and comparison of sequences from these different species, which are likely involved and critical for activity of CYB5 and/or CYP17 and the 16-androstene steroid pathway. This includes residues in the steroid binding pocket of CYP17 and residues on the surface of CYP17 that are involved in binding to CYB5.

Boar taint, an unpleasant odor and flavor emanating from meat originating from intact male pigs, is caused by the accumulation of androstenone and skatole in the fat. Castration of young boars is a common practice and is effective in preventing boar taint. Castration without any anesthesia induces considerable pain, such that stress response and animal welfare are also a concern (Gunn et al., 2004). In fact, some countries have already banned castration and large retailers in Europe have announced that they will no longer accept pork from castrated males. Castration of male pigs has been banned in several EU countries due to animal welfare concerns and a total ban on castration will take effect in the EU in 2018. Thus, high levels of boar taint from intact males are an increasing concern which is emerging in international markets and it is only a matter of time before this will be a factor in North American markets.

Androstenone is a 16-androstene steroid produced in the testis as the boar nears puberty, and it acts as a sex pheromone to regulate reproductive development in gilts and induce a mating stance in sows (Gower, 1972). Androstenone is also highly lipophilic, so it accumulates in the adipose tissue (Claus et al., 1971) leading to the disagreeable boar taint odor and flavor of meat from uncastrated male pigs.

The pivotal direct cleavage step in the biosynthesis of 16-androstene steroids from progestogens to form 5,16-androstadien-3β-ol (ANβ; 16A steroid), is catalyzed by cytochrome P450C17 (CYP17A1). This enzyme also catalyzes the 17α-hydroxylation of progestogens and the subsequent C17,20 lyase reaction leading to the biosynthesis of sex steroids (Lee-Robichaud et al., 2004), a process unrelated to boar taint but vital for the superior growth performance of intact boars. The other components of the cytochrome P450 system, P450 oxidoreductase (POR), cytochrome b5 (CYB5) and cytochrome b5 reductase (CYB5R3), affect which of the three reactions catalyzed by CYP17 predominates. An increased level of POR stimulates the lyase activity to increase androgen production (Auchus and Miller, 1999). CYB5 interacts allosterically with the CYP17-POR complex to stimulate the lyase activity as well as the synthesis of 16-androstene steroids (Yamazaki et al., 1998). However, the lyase reaction is less dependent on CYB5, while the synthesis of the 16-androstene steroids requires CYB5 (Meadus et al., 1993). A rare polymorphism in the porcine CYB5 gene has been identified just upstream of the translational start site that results in decreased production of CYB5 and decreased synthesis of androstenone (Peacock et al., 2008). CYB5 also interacts with a number of other CYP450 isoforms; a CYB5 knockout mouse model has a dramatically altered expression of drug metabolizing enzymes and low levels of testicular androgens (McLaughlin et al., 2010). Therefore, totally eliminating the expression of CYB5 would result in decreased synthesis of androgens as well as decreased synthesis of androstenone.

### Mutations in CYP17A1

### Active Site Directed

The steroid binding pocket of CYP17 includes two regions, amino acids 101-102 and 111-116 (Swart et al., 2010; shown in bold in FIG. 1).

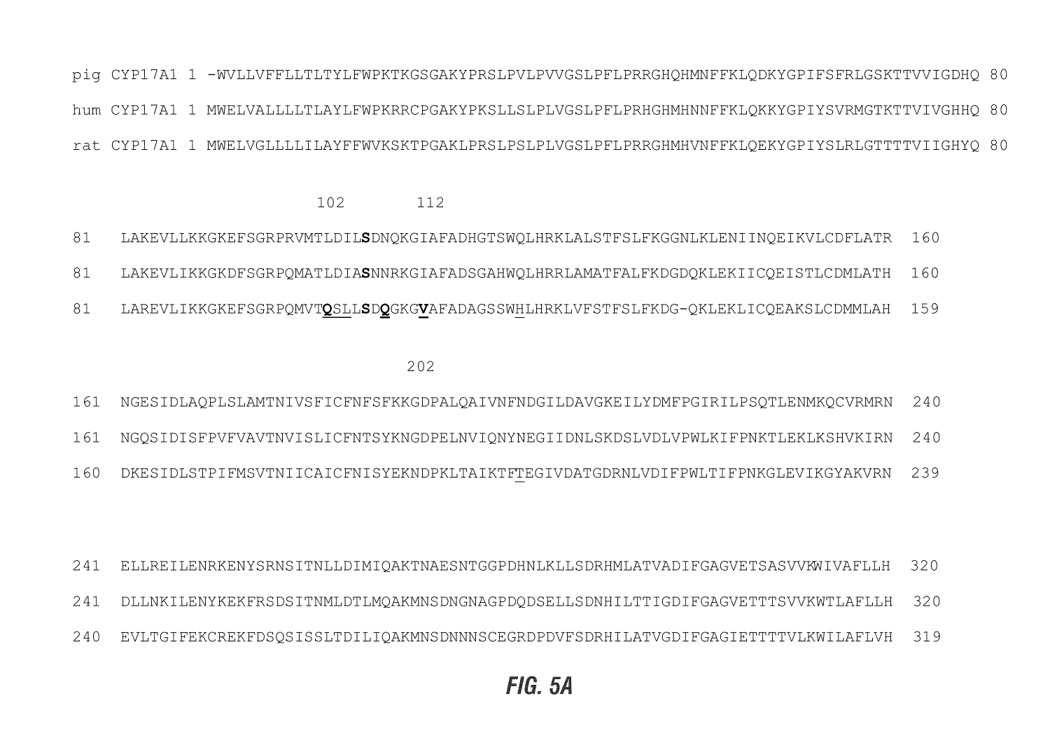

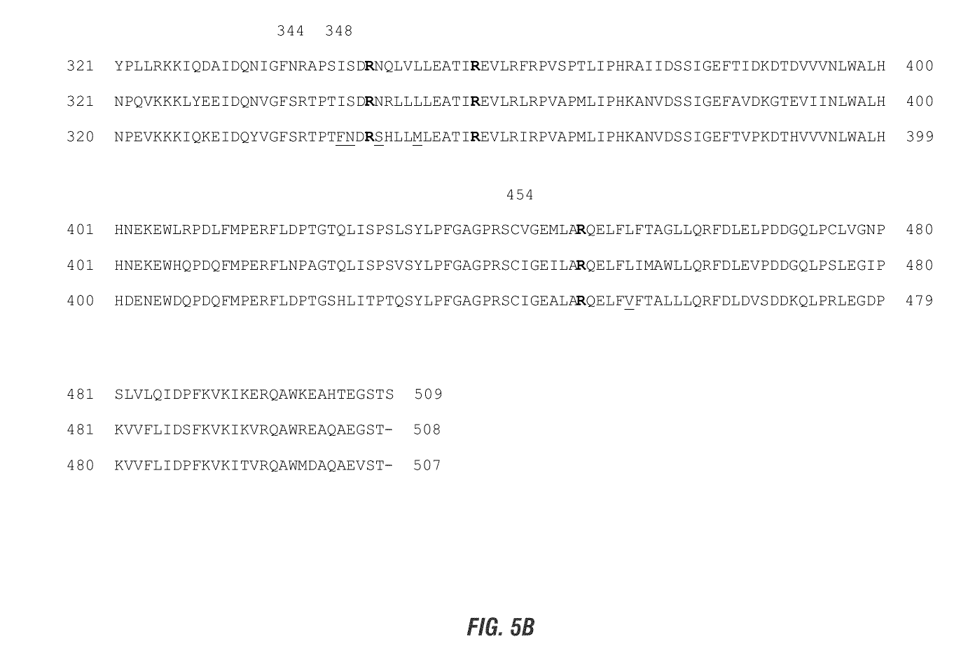

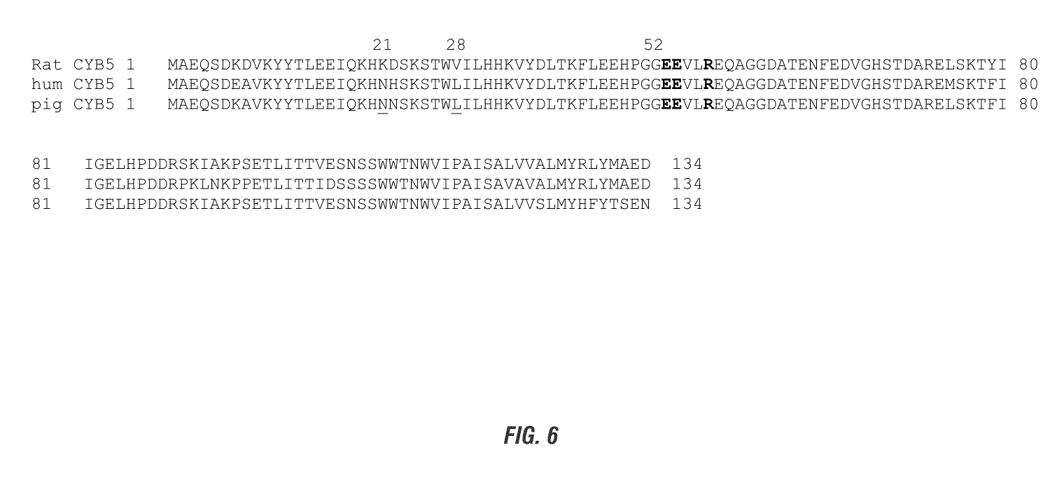

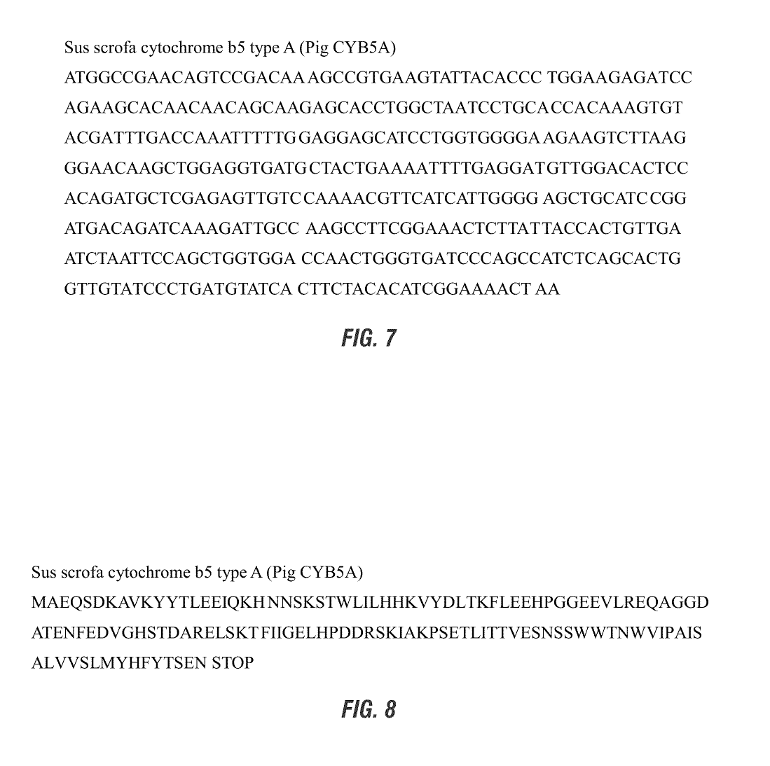

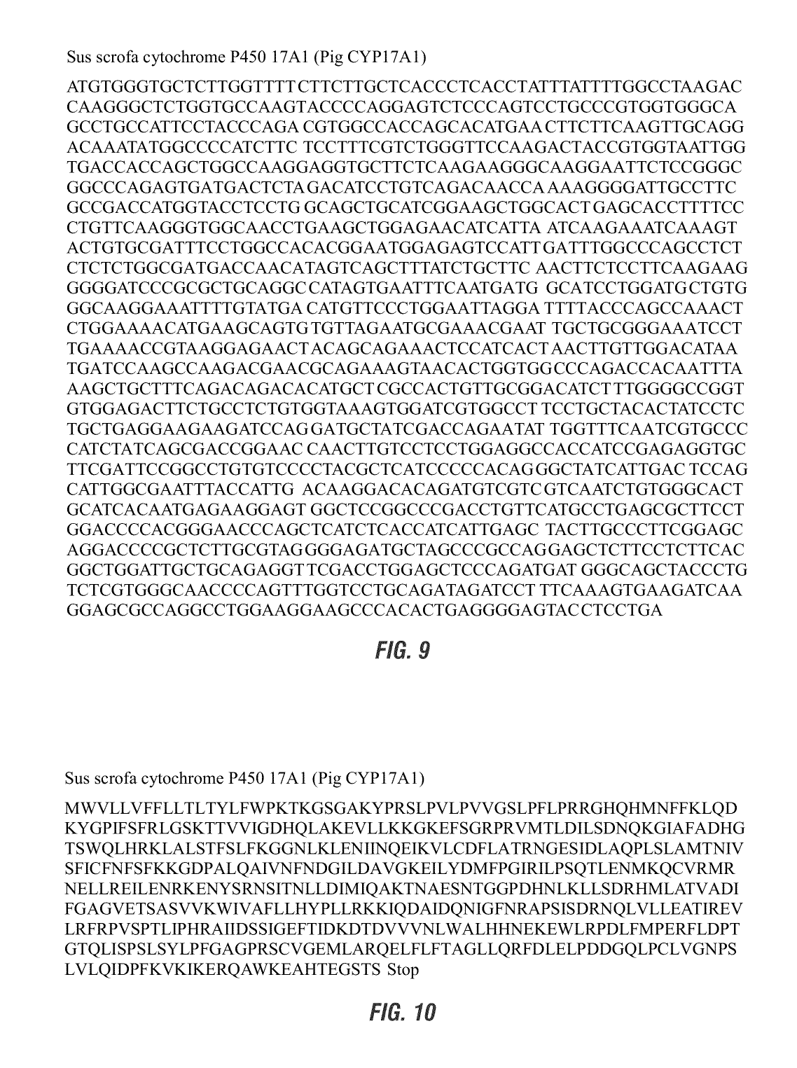

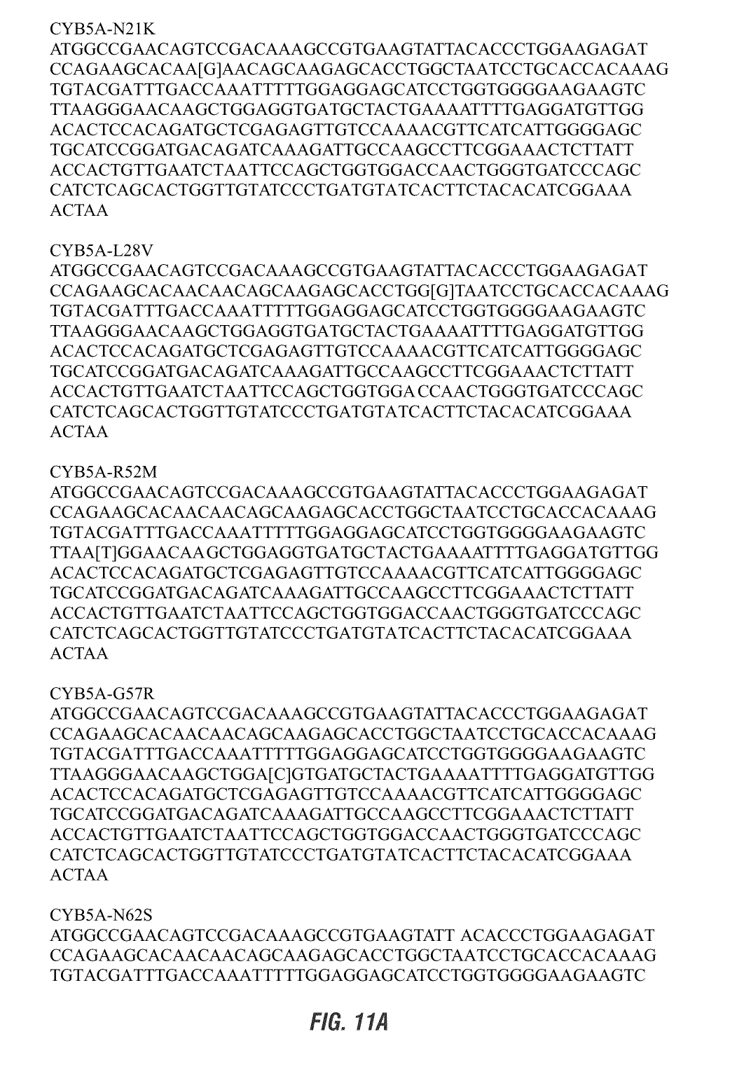

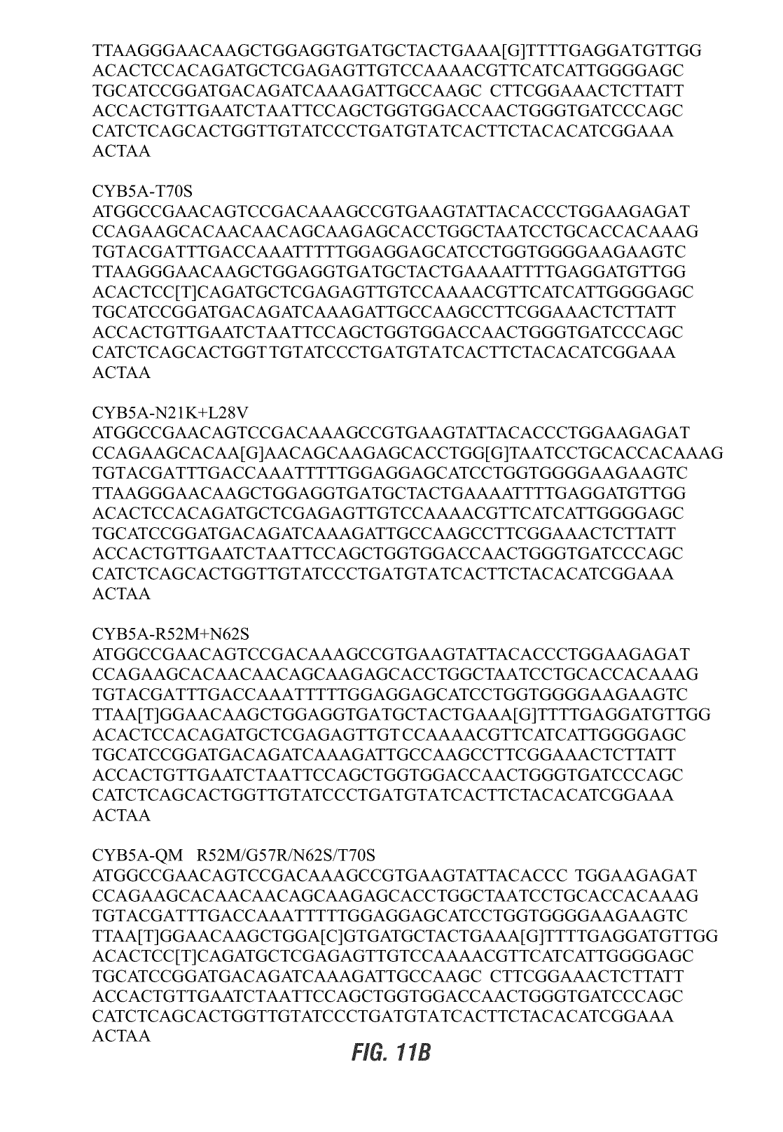

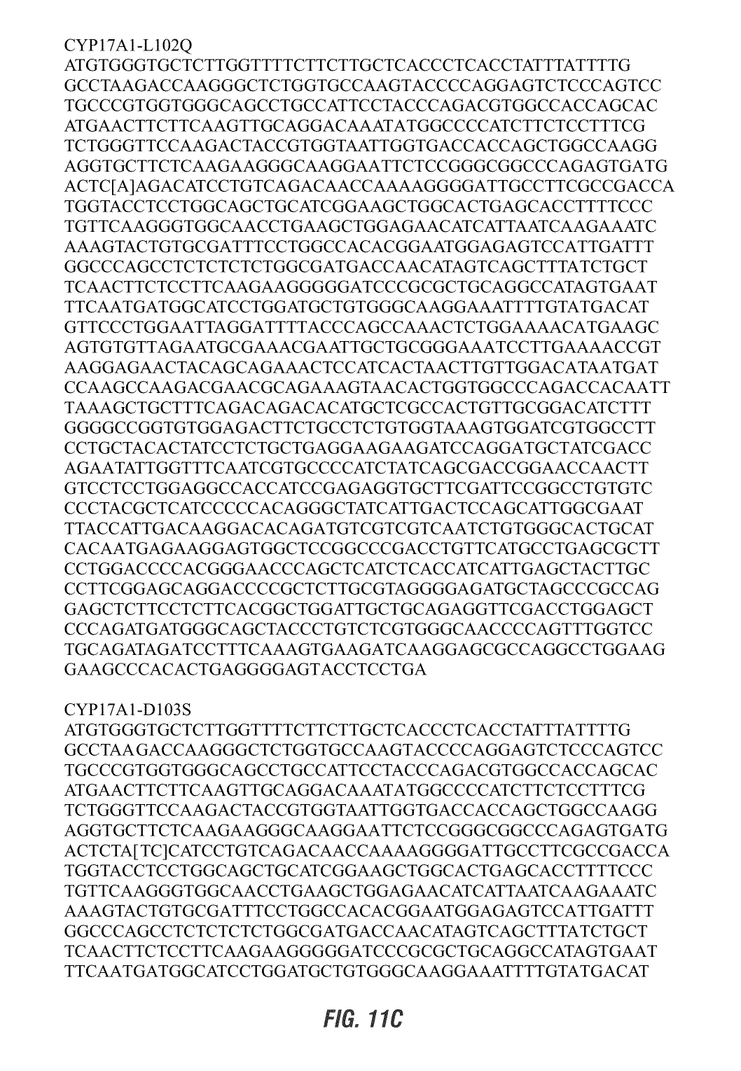

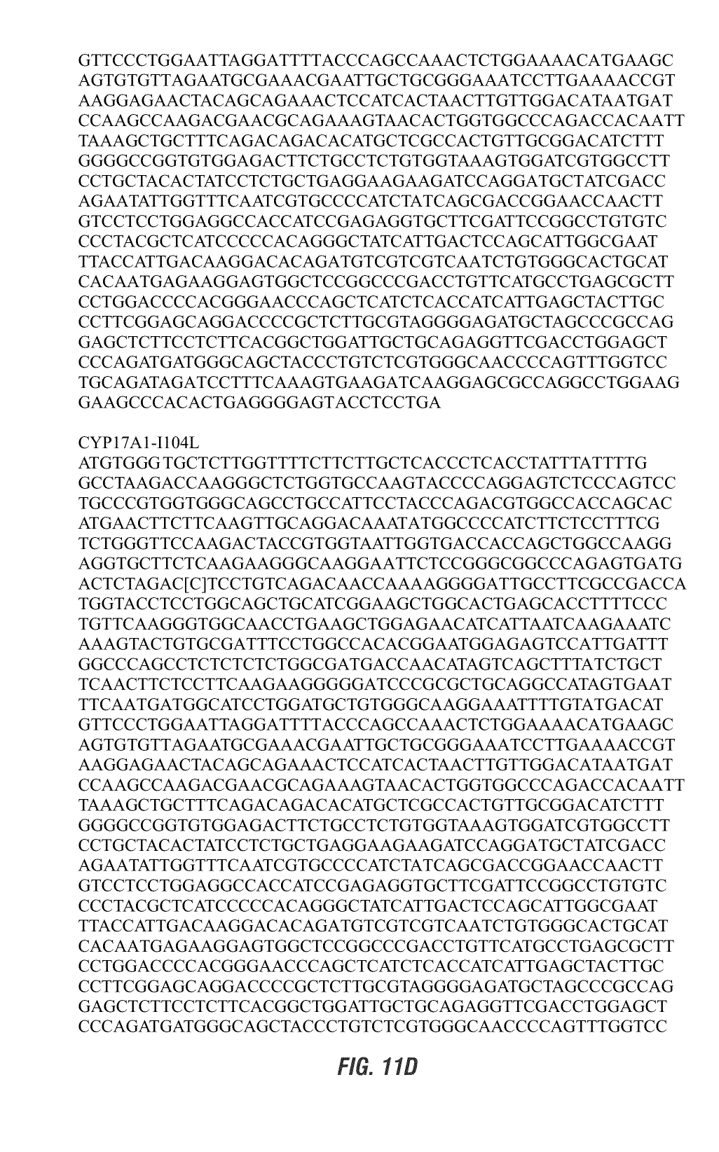

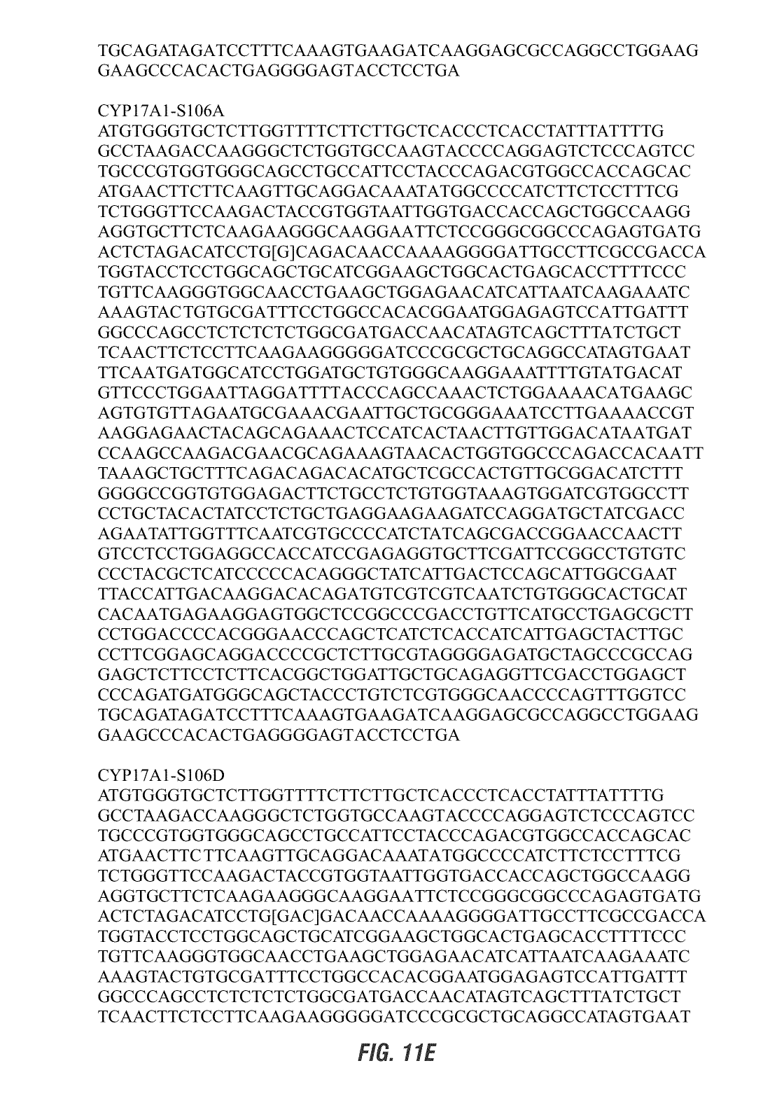

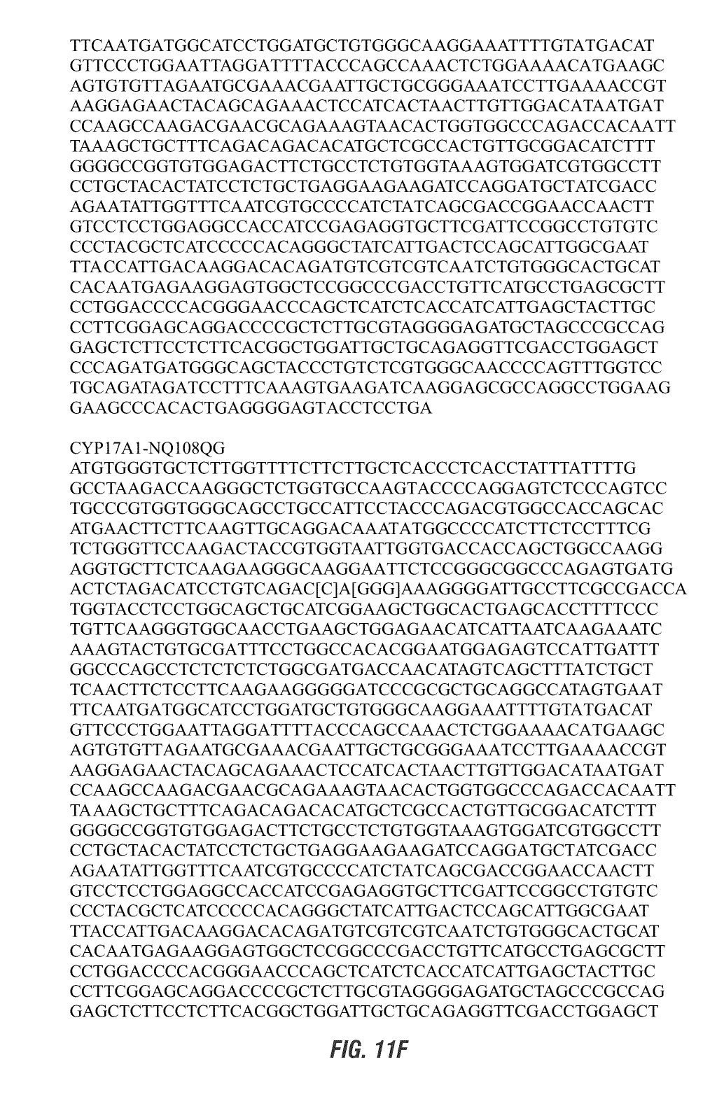

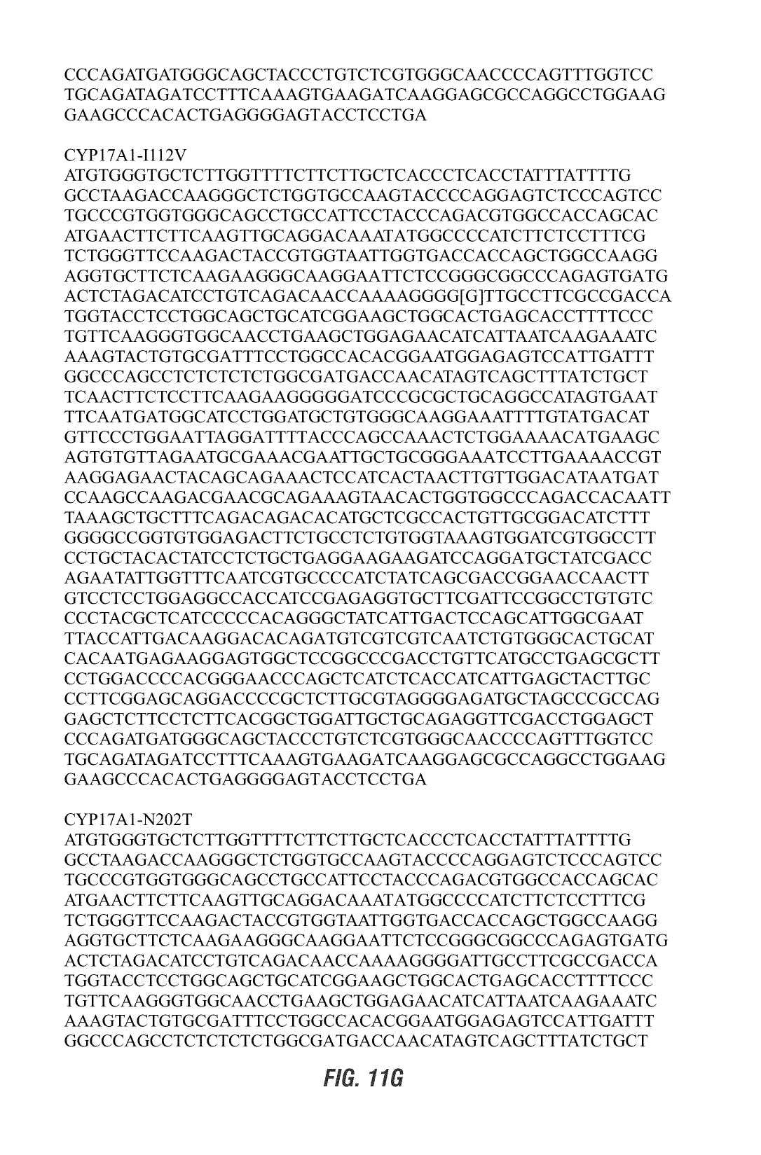

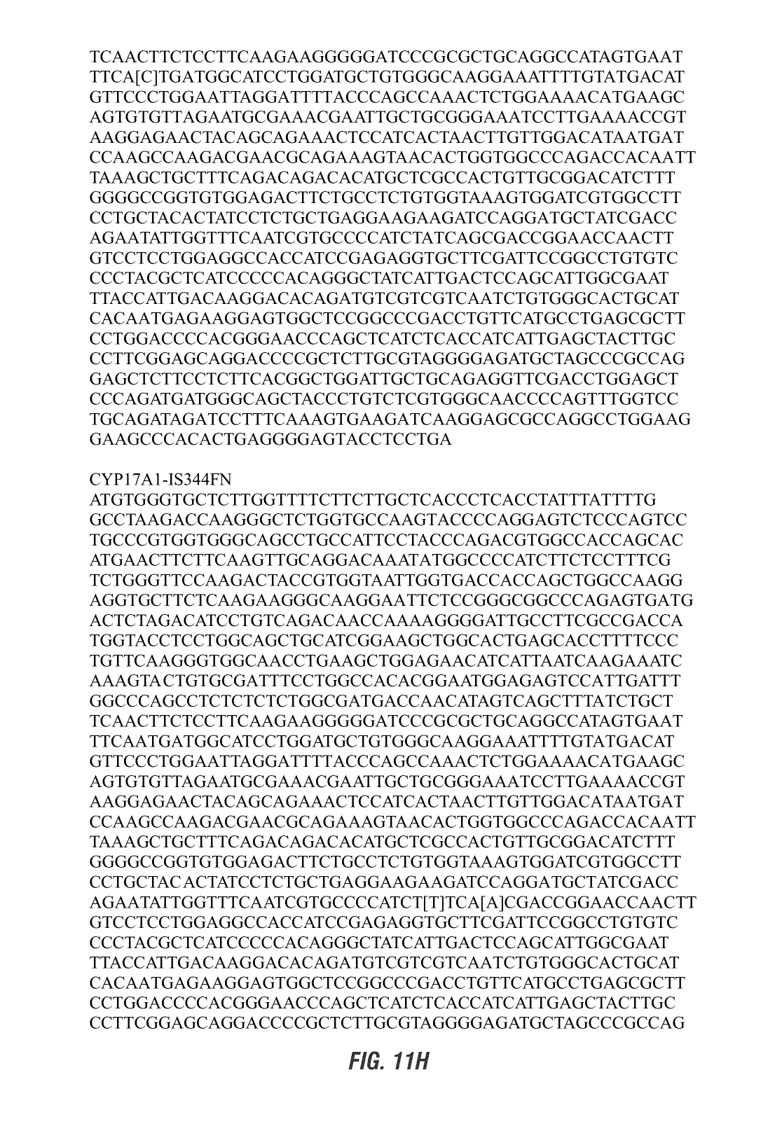

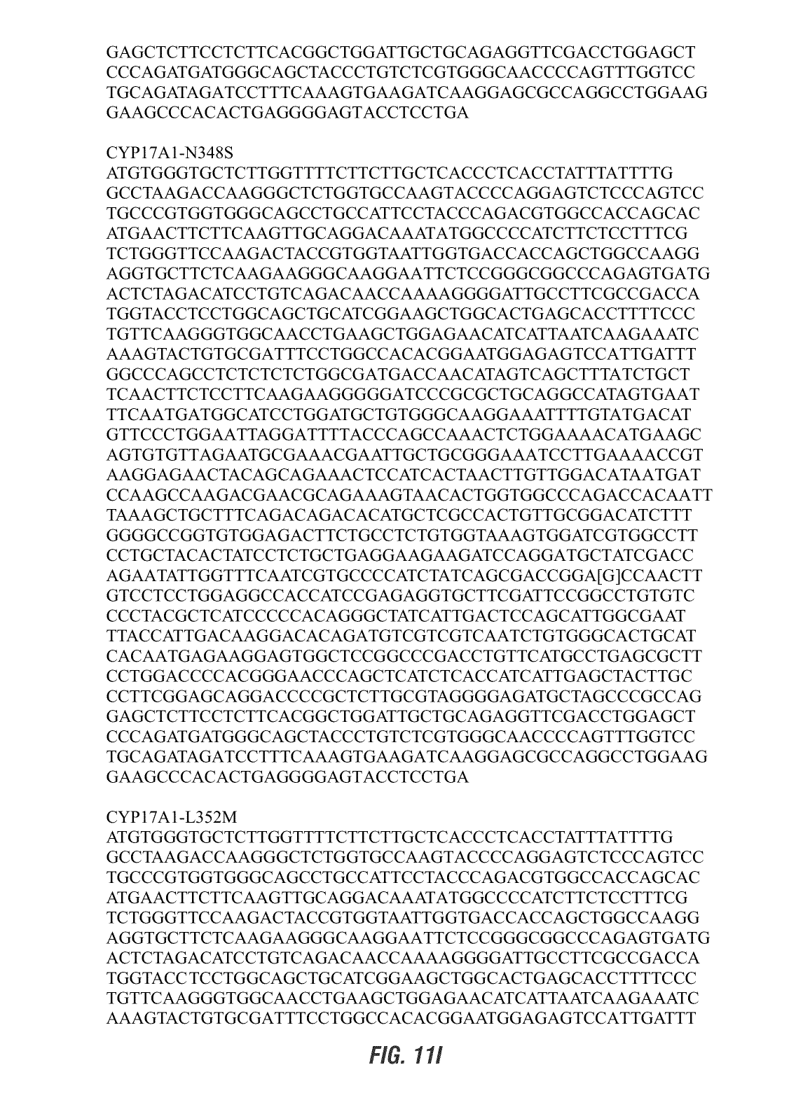

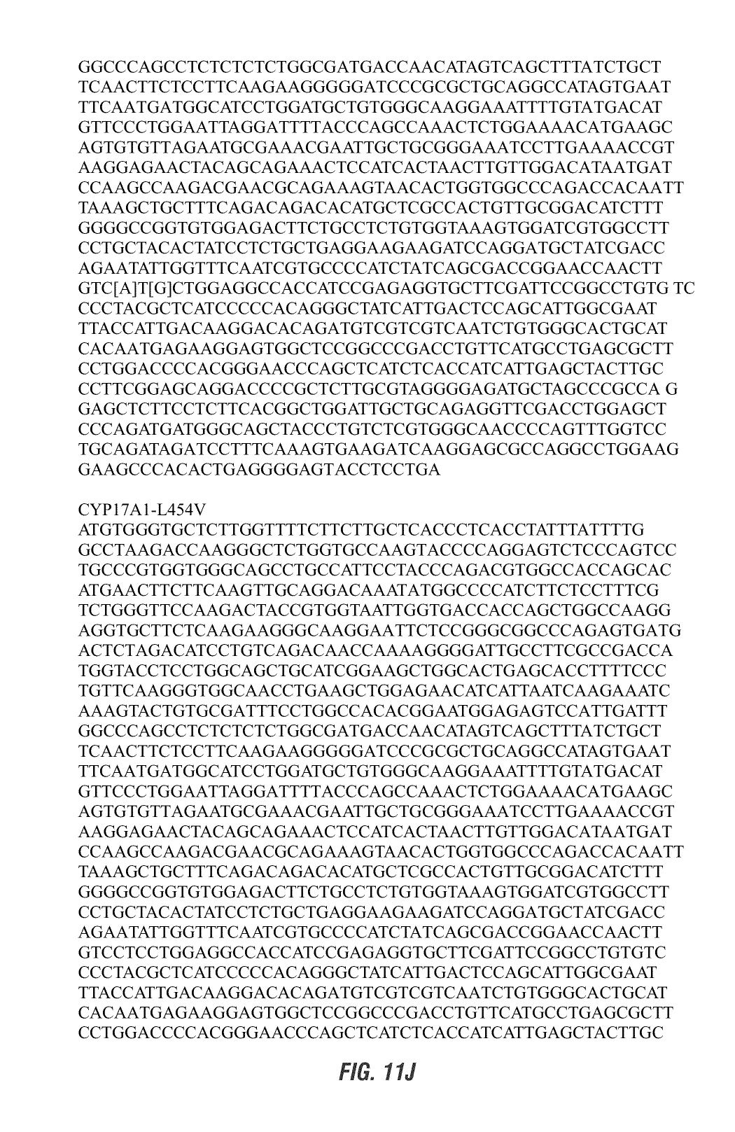

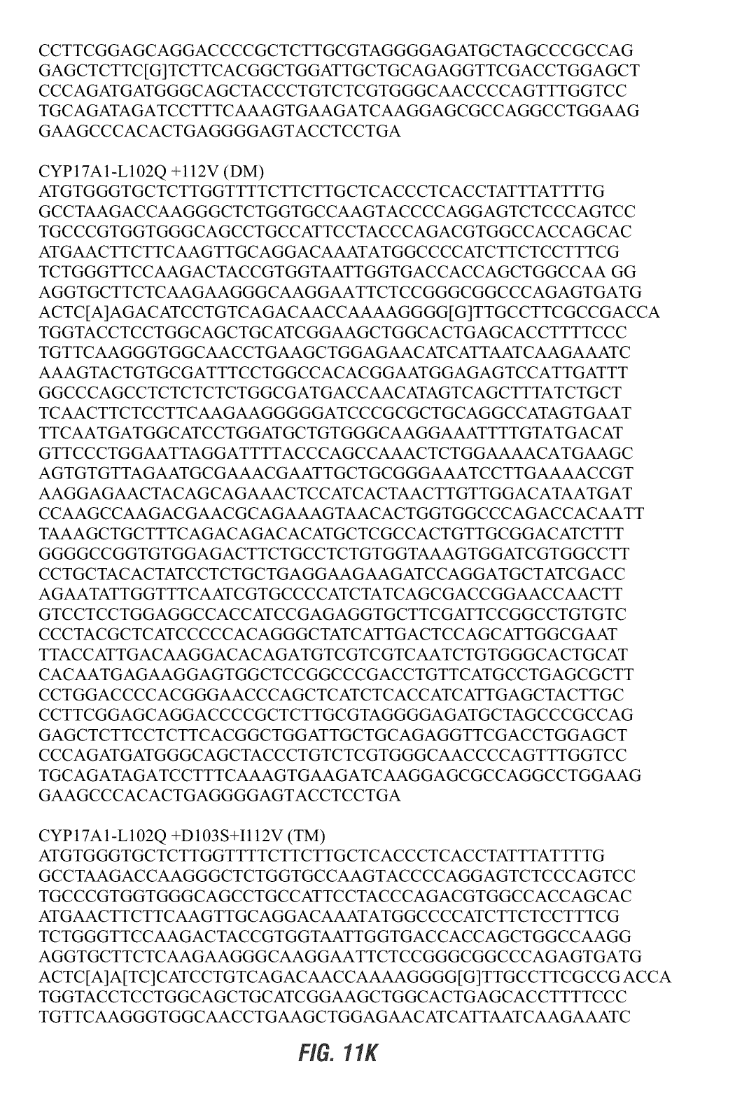

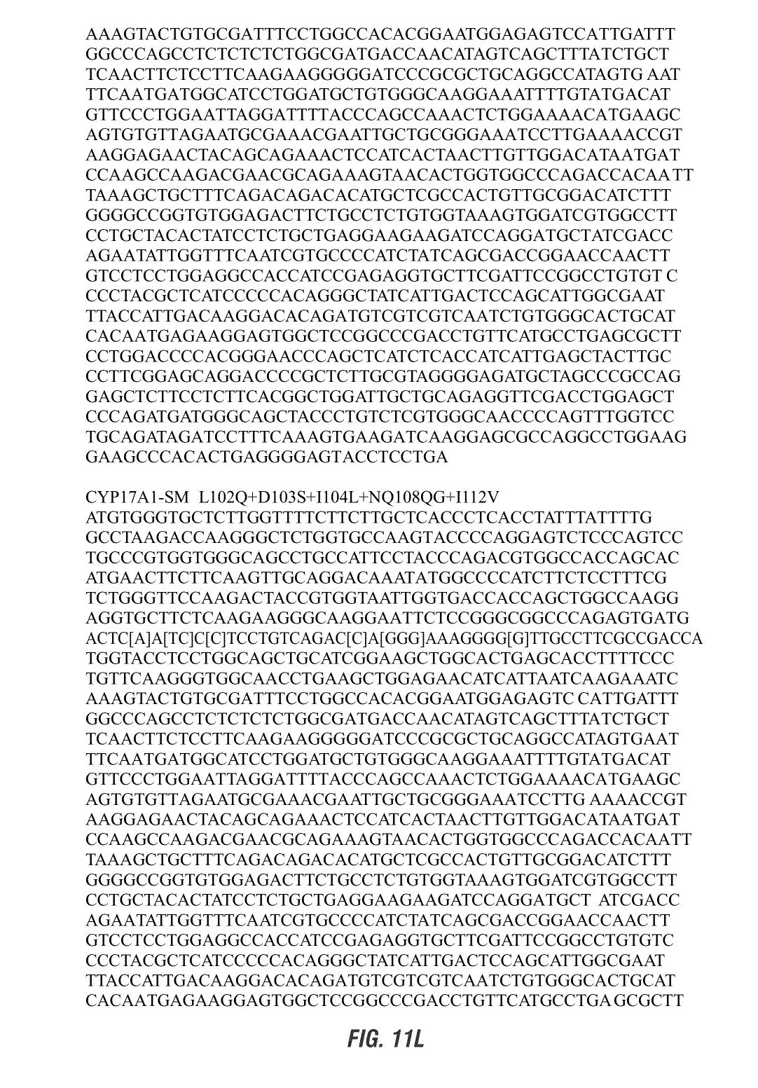

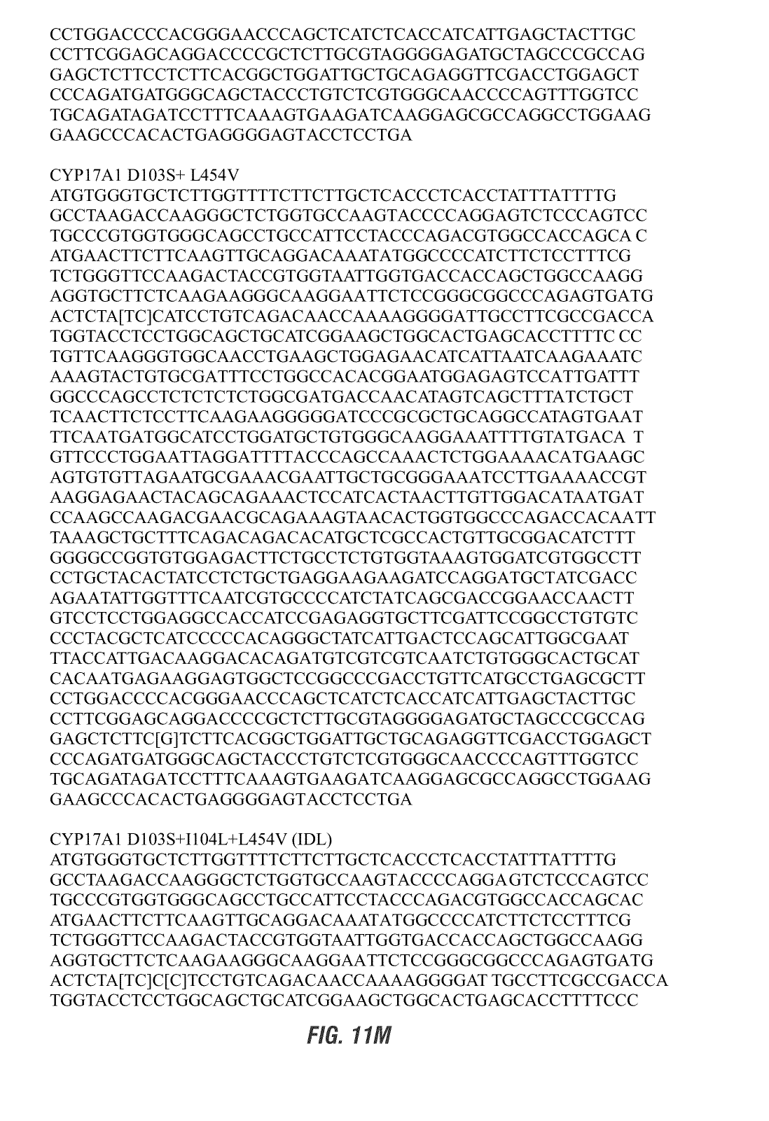

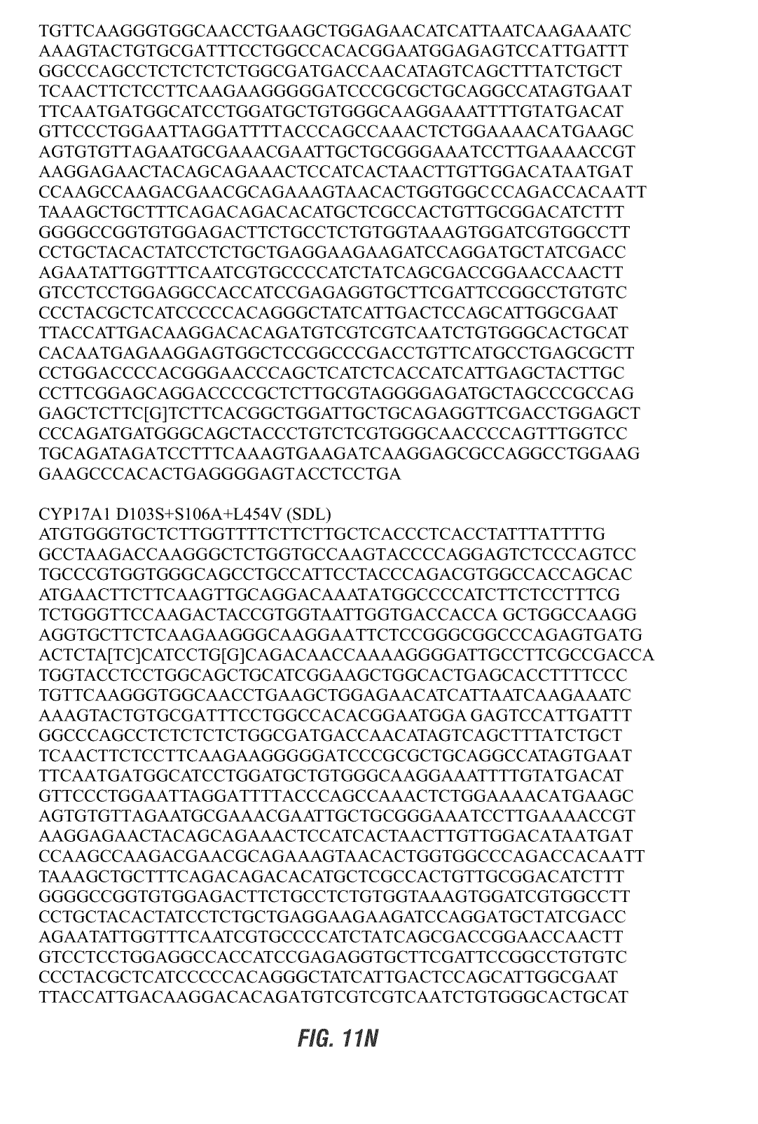

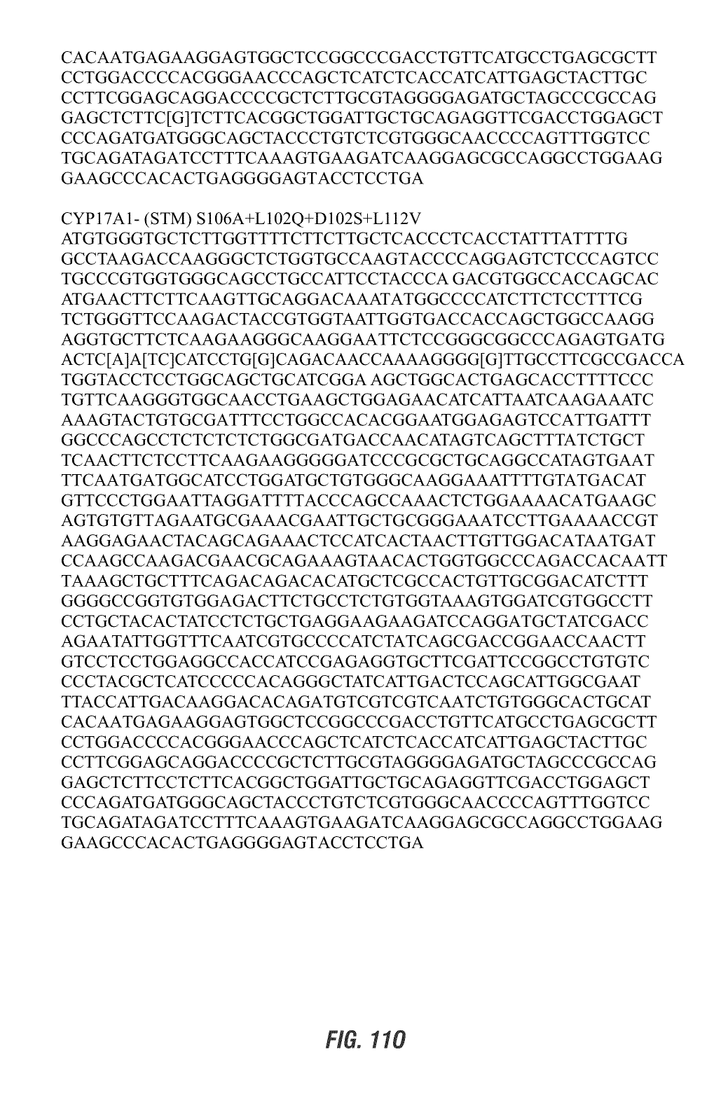

There have been no reported mutations in CYP17 that differentially affect the C17,20 lyase reaction from the formation of the 16-androstenes. However, rat testis does not form 16-androstenes (Cooke and Gower, 1977) and rat CYP17 differs by having glutamine at position 102 and valine at position 112 compared to leucine at position 102 and isoleucine at position 112 in porcine and human CYP17, which both catalyse the formation of 16-androstenes. This suggests that mutations L102Q and I112V might decrease the formation of 16-androstenes by porcine CYP17 while not adversely affecting the lyase and hydroxylase activities. Other residues in this region may also be good candidates for mutation, especially those that are near the critical residues 102 and 112. This includes residues 103, 104, 108, 109 and 122 that are different in rat compared to human and porcine CYP17, suggesting the mutations D103S, I104L, NQ108QG, and Q122H. Other residues that form part of the three dimensional structure of the steroid binding pocket of CYP17 would also be good candidates. This includes residue 202 which is involved in positioning of the substrate in the active site (DeVore and Scott, 2012); this residue is asparagine in pig and human CYP17A1 but is a threonine in rat CYP17A1 (FIG. 5), suggesting the mutation N202T. Swart et al (2010) have shown that mutation of leucine 105 in pig CYP17 to alanine (as is found in human CYP17) dramatically decreases the C17,20 lyase activity, increases the 17α-hydroxylase activity and eliminates the stimulatory effect of CYB5. The conserved serine 106 is required for both the lyase and hydroxylase activities.

### Binding of CYP17 and CYB5

A model of human CYP17 (Auchus and Miller, 1999) can be used to predict the amino acid residues that are involved in binding of CYP17 to CYB5. Mutation of either R347, R358 or R449 to alanine in human CYP17 prevented binding of CYB5 and eliminated the C17,20 lyase activity and the formation of the 16-androstene steroids, with no effect on the hydroxylase activity (Lee-Robichaud et al., 2004). The recent structural determination of human CYP17A1 that is available at NCBI (DeVore and Scott, 2012) illustrates that these residues are on the proximal face of the protein, which is consistent with their proposed role in CYB5 binding. A comparison of the sequences of human, pig and rat CYP17A1 in these regions identifies key residues that are different in rat than in human or pig. These candidates for mutation are I344F, S345N (or IS344FN), N348S and L352M in the region of R347 and R358 (FIG. 2A) and L454V in the region of R449 (FIG. 2B).

Phosphorylation of serine and threonine residues of human CYP17 increases the lyase activity with no effect on the 17α-hydroxylation reaction (Pandley and Miller, 2005). Our own unpublished work has shown that altering the phosphorylation status of porcine CYP17 in the presence of CYB5 can increase the lyase activity and at the same time decrease the synthesis of 16-androstene steroids. Thus, altering the phosphorylation status of porcine CYP17 is a potential method for decreasing the synthesis of androstenone while maintaining the synthesis of androgens and estrogens. However, while many potential serine/threonine phosphorylation sites have been investigated in human CYP17 (Wang et al., 2010), the site of phosphorylation that affects the lyase activity has not yet been identified. However, the conserved S106 which is located in the steroid binding pocket may be important. Mutation S106A mimics the unphosphorylated form of CYP17 and S106D mimics the phosphorylated form of CYP17.

### CYB5

CYB5 has many roles such as: (a) transfer of electrons from NADH to desaturase (Ozols, 1976), (b) NADH-dependent reduction of methemoglobin to regenerate hemoglobin (Abe and Sugita 1979), and (c) stimulation of cytochrome P450 dependent oxygenation (Ogishima et al, 2003). Experiments with apo-CYB5, which lacks the heme moiety, and holo-CYB5 suggest that CYB5 is not responsible for direct electron transfer but exerts a saturable, allosteric effect on the CYP17A1-POR complex (Yamazaki et al., 1998). The POR functions by catalyzing electron transfer from NADPH to cytochrome P450 during catalysis (Lu and West, 1978) and is also involved in electron transfer from NADPH to heme oxygenase (Yoshida and Kikuchi, 1978) and CYB5 (Ilan et al., 1981).

There are two forms of CYB5, the microsomal CYB5A that is involved in regulating steroidogenesis and the so-called ‘outer mitochondrial’ CYB5B. We have shown that while CYB5A stimulates both the lyase activity and formation of the 16-androstene steroids by porcine CYP17, CYB5B only stimulates the lyase reaction but has no effect on the synthesis of 16-androstene steroids (Billen and Squires, 2009). Mutational studies with CYB5 have identified the region on the surface of human CYB5 that is involved in binding of human CYB5A to the CYP17-POR complex. Mutation of amino acid residues E48, E49 and R52 to glycine reduced the stimulation of C17,20 lyase activity by CYB5A but did not affect the capacity of the protein to bind heme or accept electrons from POR (Naffin-Olivod and Auchus, 2006). Comparing the region between CYB5A and CYB5B (FIG. 3), it can be seen that while E48 and E49 are conserved, arginine 52 is a methionine in CYB5B; this suggests that mutation R52M in CYB5A may shift the activity of CYB5A to be more like CYB5B. Other residues in this region might also be good candidates for mutation, such as residues 57, 62 and 70 which differ between CYB5A and CYB5B; this suggests mutations G57R, N62S and T70S (FIG. 3).

Other residues that would affect the three dimensional structure that interacts with the CYP17-POR complex would also be good candidates for mutagenesis. A comparison of the sequence of CYB5A between rat, human and pig (FIG. 4) identifies residues 21 and 28 that are lysine and valine in rat compared to asparagine and leucine in human and pig. Using the three dimensional structure of CYB5 available at NCBI (Banci et al., 1997), these residues are located in the same region as residues 48, 49 and 52 that is involved in binding of CYB5 to CYP17. This suggests that mutations N21K and L28V may reduce the synthesis of the 16-androstene steroids while maintaining the normal production of sex steroids. Other amino acids in this region, including D58, D65 and E74 are required for effective stimulation of lyase activity (Naffin-Olivod and Auchus, 2006).

Among the several systems developed to characterize enzymatic activity, transfection of expression constructs into intact mammalian cells provides the opportunity to study the activity of the expressed proteins in the native microsomal environment and to study various combinations of enzyme, redox partners and substrate (Dufort et al., 1999; Luu-The et al., 2005; Billen and Squires, 2009). In the present work, we have studied the effects of mutation of specific amino acid residues in CYB5A and CYP17A1 on the formation of 17OHP, DHEA and 16A steroids by transient transfection of human embryonic kidney (HEK-293) cells with expression constructs for POR and CYB5R3 and various mutants of CYB5A and CYP17A1. With this system we have the ability to over-express the proteins of interest, and to vary the relative amounts of these proteins to produce a well-defined and active system with a minimum of interference from endogenous proteins. Our objective was to determine how specific amino acid residues in CYB5A and CYP17A1 modulate the three activities of porcine CYP17A1; 17α-hydroxylase, C17,20 lyase and 16A steroid synthesis activity.

### Methodology:

The experimental approach is to identify and mutate the residues on porcine CYB5A and CYP17A1 that are necessary for 16-androstene steroid synthesis. We then test the effect of the mutations in CYB5 and CYP17 in the in vitro system in which we express porcine POR, CYB5R3 and various mutants of CYB5A and CYP17A1 in HEK273 cells (Billen and Squires, 2009) and measure the formation of metabolites from radiolabelled pregnenolone by HPLC. This will allow us to rapidly screen for those amino acid changes that reduce the formation of the 16-androstene steroids but do not adversely affect the 17α-hydroxylase and C17,20 lyase reactions that are necessary for the synthesis of sex steroids.

### Construction of Expression Vectors for POR, CYP17A1, CYB5R3 and CYB5A

The entire coding regions of porcine NADPH cytochrome P450 reductase (POR), cytochrome P450 C17 (CYP17A1), cytochrome b5 reductase (CYB5R3) and cytochrome b5A (CYB5A) were amplified from porcine testis cDNA by PCR using platinum Pfx DNA polymerase (Invitrogen) and appropriate primers (Billen and Squires, 2009). The amplified segments were then cloned into pcDNA3.1/V5-His TOPO (Invitrogen) to produce expression vectors. Expression vectors for V5-His tagged proteins were generated so that the expressed proteins could be detected by Western blotting using anti-V5-HRP antibody; vectors expressing the untagged protein were also generated to determine if the V5-His tag adversely affected the activity of the proteins. The PCR primers for porcine NADPH cytochrome P450 reductase (POR) were based on Genebank accession number L33893, the primers for porcine CYP17A1 were based on accession number NM 214428 and the primers for porcine CYB5A were based accession number NM 001001770. The sequence of porcine CYB5R3 was assembled from pig ESTs retrieved by BLAST searching the NCBI database using human CYB5R3 (accession number NM 000398) as a template. This sequence was used to design primers to amplify and clone porcine CYB5R3 (Billen and Squires, 2009). The identity of all clones was confirmed by sequencing.

### Site Directed Mutagenesis

Generation of CYP17A1 and CYB5A mutants was carried out using the Change-ITTM Multiple Mutation Site Directed Mutagenesis Kit (USB Corporation, Cleveland, Ohio) following the manufacturer's instructions. The protocol for all mutagenic PCRs was as follows, unless otherwise noted: (95° C. 2 min [95° C. 30 sec, 62° C. 30 sec, 68° C. 20 min]×35 cycles, 68° C. 10 min). All mutations were carried out using the AMP-F primer listed on Table 1 as a common reverse primer, unless otherwise noted. Individual CYP17A1 (-L102Q, -D103S, -I104L, -Q108QG, or -I112V) mutants were generated from the CYP17A1-WT plasmid using the mutagenic primers from Table 1. CYP17A1-L102Q/I112V was generated by mutating CYP17A1-L102Q with the -I112V primer. CYP17A1-LD102QS+I112V (-TM) was generated by mutating CYP17A1-L102Q/I112V with the -LD102QS primer. The CYP17A1-Sextuple mutant (-SM) (containing all individual mutations) was generated by first mutating CYP17A1-LD102QS/I112V with the -Quintuple primer, then mutating the resulting plasmid with the -Sextuple primer. Individual CYB5A mutants (-R52M, -G57R, -N62S, or -T70S) were generated from CYB5A-WT using the primers listed in Table 1. CYB5A-R52M/N62S was generated by mutating CYB5A-R52M with the -N62S primer. CYB5A-Quintuple mutant (-QM) was generated by mutating CYB5A-R52M/N62S with the -T70S primer, then mutating the resulting plasmid with the -Triple primer. CYP17A1 phosphorylation mutations (-S106D phosphor-mimic or -S106A dephosphor-mimic) were also generated using CYP17A1-WT as template. CYP17A1-S106D was generated using the -S106D primer with the Chang-ITTM kit as described above. CYP17A1-S106A was generated using the QuickChange® Site-Directed Mutagenesis Kit (Strategene, La Jolla, Calif.) following the manufacturer's instructions. The CYP17A1-S106A-F and -S106A-R primers listed on Table 1 were used, with cycling conditions as follows: (95° C. 30 sec [95° C. 30 sec, 55° C. 1 min, 68° C. 7 min]×16 cycles, 68° C. 10 min). CYP17A1-S106A+TM (-STM) was generate using the QuickChange® Site-Directed Mutagenesis Kit with CYP17A1-TM as the template, using the CYP17A1-S106A/TM-F and CYP17A1-S106A/TM-R primers listed below. The CYP17A1-D103S+S106A+L454V and CYP17A1-D103S+I104L+L454V plasmids were generated using the QuickChange® Site-Directed Mutagenesis Kit with CYP17A1-L454V plasmid as a template, with the primers CYP17A1-S106A/D103S-F and CYP17A1-S106A/D103S-R or CYP17A1-I104L/D103-F and CYP17A1-I104L/D103-R, respectively. The cycling parameters for all of these mutations were the same as for generation of CYP17A1-S106A.

TABLE 1
Primers used for mutagenesis

| Primer Name | Sequence |
|---|---|
| CYP17A1-L102Q | 5′PO₄-CCAGAGTGATGACTCAAGACATCCTGTCAG-3′ |
| | (SEQ ID NO: 51) |
| CYP17A1-D103S | 5′PO₄-CAGAGTGATGACTCTATCCATCCTGTCAGACAACC-3′ |
| | (SEQ ID NO: 52) |
| CYP17A1-I104L | 5′PO₄-GTGATGACTCTAGACCTCCTGTCAGACAAC-3′ |
| | (SEQ ID NO: 53) |
| CYP17A1-NQ108QG | 5′PO₄-CTAGACATCCTGTCAGACCAGGGAAAGGGGATTGCCTTCGC-3′ |
| | (SEQ ID NO: 54) |
| CYP17A1-I112V | 5′PO₄-GACAACCAAAAGGGGGTTGCCTTCGCCGAC-3′ |
| | (SEQ ID NO: 55) |
| CYP17A1-LD102QS | 5′PO₄-CAGAGTGATGACTCAATCCATCCTGTCAGACAACC-3′ |
| | (SEQ ID NO: 56) |
| CYP17A1-Quintuple | 5′PO₄-CAATCCATCCTGTCAGACCAGGGAAAGGGGGTTGCCTTCGC-3′ |
| | (SEQ ID NO: 57) |
| CYP17A1-Sextuple | 5′-PO₄-GTGATGACTCAATCCCTCCTGTCAGACCAG-3′ |
| | (SEQ ID NO: 58) |
| CYP17A1-S106D | 5′PO₄-GATGACTCTAGACATCCTGGACGACAACCAAAAGGGGATTG-3′ |
| | (SEQ ID NO: 59) |
| CYP17A1-S106A-F | 5′-ACTCTAGACATCCTGGCAGACAACCAAAAGGGG-3′ |
| | (SEQ ID NO: 60) |
| CYP17A1-S106A-R | 5′-CCCCTTTTGGTTGTCTGCCAGGATGTCTAGAGT-3′ |
| | (SEQ ID NO: 61) |
| CYP17A1-N202T | 5′PO₄-CCATAGTGAATTTCACTGATGGCATCCTGG-3′ |
| | (SEQ ID NO: 62) |
| CYP17A1-IF344FN | 5′PO₄-GTTTCAATCGTGCCCCATCTTTCAACGACCGGAACCAACTTGTC-3′ |
| | (SEQ ID NO: 63) |
| CYP17A1-N348S | 5′PO₄-CTATCAGCGACCGGAGCCAACTTGTCCTCC-3′ |
| | (SEQ ID NO: 64) |
| CYP17A1-L352M | 5′PO₄-CGGAACCAACTTGTCATGCTGGAGGCCACCATC-3′ |
| | (SEQ ID NO: 65) |
| CYP17A1-L454V | 5′PO₄-CAGGAGCTCTTCGTCTTCACGGCTG-3′ |
| | (SEQ ID NO: 66) |
| CYP17A1- | 5′PO₄-GATGACTCAATCCATCCTGGCAGACAACCAAAAGGGGGTTG-3′ |
| S106A/TM-F | (SEQ ID NO: 67) |
| CYP17A1- | 5′PO₄-CAACCCCCTTTTGGTTGTCTGCCAGGATGGATTGAGTCATC-3′ |
| S106A/TM-R | (SEQ ID NO: 68) |
| CYP17A1- | 5′PO₄-GATGACTCTATCCATCCTGGCAGACAACCAAAAGGGGATTG-3′ |
| S106A/D103S-F | (SEQ ID NO: 69) |
| CYP17A1- | 5′PO₄-CAATCCCCTTTTGGTTGTCTGCCAGGATGGATAGAGTCATC-3′ |
| S106A/D103S-R | (SEQ ID NO: 70) |
| CYP17A1- | 5′PO₄-GTGATGACTCTATCCCTCCTGTCAGACAAC-3′ |
| I104L/D103S-F | (SEQ ID NO: 71) |
| CYP17A1- | 5′PO₄-GTTGTCTGACAGGAGGGATAGAGTCATCAC-3′ |
| I104L/D103S-R | (SEQ ID NO: 72) |
| CYB5A-R52M | 5′-PO₄-GGAAGAAGTCTTAATGGAACAAGCTGGAGG-3′ |
| | (SEQ ID NO: 73) |
| CYB5A-G57R | 5′PO₄-GTCTTAAGGGAACAAGCTGGACGTGATGCTACTGAAAATTTTG-3′ |
| | (SEQ ID NO: 74) |
| CYB5A-N62S | 5′PO₄-GGTGATGCTACTGAAAGTTTTGAGGATGTTGG-3′ |
| | (SEQ ID NO: 75) |
| CYB5A-T70S | 5′PO₄-GATGTTGGACACTCCTCAGATGCTCGAGAG-3′ |
| | (SEQ ID NO: 76) |
| CYB5A-Triple | 5′PO₄-GTCTTAATGGAACAAGCTGGACGTGATGCTACTGAAAGTTTTG-3′ |
| | (SEQ ID NO: 77) |
| CYB5A-N21K | 5′PO₄-GATCCAGAAGCACAAGAACAGCAAGAGCAC-3′ |
| | (SEQ ID NO: 78) |
| CYB5A-L28V | 5′PO₄-CAAGAGCACCTGGGTAATCCTGCACC-3′ |
| | (SEQ ID NO: 79) |
| AMP-F | 5′PO₄-CCATGAGTGATAACACTGCGGCCAACTTACTTCTGAC-3′ |
| | (SEQ ID NO: 80) |

### Transient Expression in Human Embryonic Kidney (HEK-293) Cells

Human embryonic kidney (HEK-293) cells were plated at 7×10⁵ cells per well in 6 well culture tissue plates (VWR) and grown in Dulbecco's modified eagle's medium (Lonza) supplemented with 10% fetal calf serum, 1% non-essential amino acids, 1% sodium pyruvate, 1% pen-strep and 1% glutamine (PAA Laboratories, Etobicoke, ON) at 37° C. Once cells were 90-95% confluent, expression vectors for CYP17A1 (0.25 ug), POR (0.35 ug), CYB5R3 (0.25 ug), and CYB5A (0 up to 1.5 ug) were transfected into HEK-293 cells using LipofectAMINE 2000 (Invitrogen) according to the manufacturer's instructions. The amounts of the plasmids for expression of CYP17A1, POR and CYB5R3 used in the transfections were adjusted to give an approximately equal amount of expression of each of these proteins. Variable amounts of the expression plasmids for CYB5A were used, with empty pcDNA3.1 vector added to bring the total amount of DNA to 4 ug per well for each transfection. Control wells were transfected with 4 ug of empty vector.

### Assay of Enzymatic Activity

The metabolism of pregnenolone was measured in HEK-293 cells transiently transfected with vectors expressing CYP17A1, POR, CYB5R3, and CYB5A. At 48 hours after transfection, [7-³H(N)]-pregnenolone (30 uM, specific activity=33 uCi umol⁻¹) was added in fresh media to the 6 well culture plates. After incubation for 16 hours, the media was collected and extracted twice with 4 mL ether and the organic phases were pooled and evaporated to dryness under a stream of nitrogen. The extracts were dissolved in 85% acetonitrile:15% H2O and the radioactive steroids were separated by HPLC on a Luna 5u 250×4.60 mm reverse phase C-18 column (Phenomenex, Torrance, Calif.). The equipment consisted of a Spectra-Physics model SP8880 autosampler, a Spectra Physics model SP8800 Ternary HPLC Pump (Spectra-Physics, San Jose, Calif.) and a β-Ram model 2 radioactivity detector (IN/US Systems, Tampa Fla.). The 16-androstene steroid product (Anβ; 16A) was separated from the pregnenolone substrate and other products (17OHP and DHEA) using a mobile phase of 85% acetonitrile delivered at 1 ml/min (Sinclair et al., 1995). DHEA and 17OHP were separated using a 50% acetonitrile mobile phase (Bonneau et al., 1992). Substrates and metabolites were identified by comparison with the retention time of reference steroids (Sigma).

### Results

We determined the effects of single amino acid mutations in CYB5 and CYP17 and various combinations of these mutations on the percentage of 16A, DHEA and 17OHP formed from pregnenolone and the total conversion of pregnenolone to these metabolites. The data for the formation of metabolites (DHEA, 16A and 17OHP) for each mutant was normalized to wild type CYP17 for each level of CYB5 expression vector used. Wild type CYP17A1 and CYB5A was analyzed at the same time as the mutants. Ideal results would be 100% or higher levels of DHEA and lowest levels of 16A for a ratio of 16A/DHEA as low as possible. The total conversion (overall activity) should be maintained at 100% or higher of wild type.

Effect of Mutations in CYP17 with Different Levels of WT CYB5

### Single Mutations

The L102Q mutation has similar activity as wild type CYP17, with no differences in DHEA or 16A production or in total conversion activity. (NO EFFECT; Table 2, Panel A). The D103 mutant has higher overall activity and produces proportionally more DHEA than wild type CYP17. This results in an improvement in the 16A/DHEA ratio of 40-60% compared to wild type CYP17 (Table 2, Panels B-D). In the three replicates the D103 mutant has higher overall activity and produces proportionally more DHEA and less 16A than wild type CYP17. This results in an improvement in the 16A/DHEA ratio of 20-30% compared to wild type CYP17. (HIGH ACTIVITY AND IMPROVED RATIO; Table 2, Panels B-D). The I104L mutation has higher overall activity than wild type CYP17, with similar or slightly higher production of DHEA and 16A, with no effect on the 16A/DHEA ratio. (HIGH ACTIVITY; Table 2, Panel E). The S106A mutant has higher overall activity and produces higher levels of 16A and DHEA than wild type CYP17. The 16A/DHEA ratio for this mutant is better than for wild type CYP17. (HIGH ACTIVITY; Table 2, Panel F). The S106D mutant has much lower production of both DHEA and 16A with higher production of 17OHP than wild type CYP17. Overall activity is much lower than for wild type CYP17. (LOW OVERALL LYASE ACTIVITY; Table 2, Panel G). The NQ108QG mutant has much higher overall activity and produces higher levels of 16A and DHEA than wild type CYP17. The 16A/DHEA ratio for this mutant is improved compared to wild type CYP17. (HIGH ACTIVITY; Table 2, Panel H). The I112V mutation severely decreases both DHEA and 16A production compared to wild type CYP17, with some indication that the 16A/DHEA ratio is improved. However, the total activity is dramatically reduced compared to wild type CYP17. (LOW ACTIVITY WITH IMPROVED RATIO; Table 2, Panel I). The L454V CYP17 mutant produces dramatically lower amounts of 16A and lesser amounts of DHEA to improve the 16A/DHEA ratio by 40-60%. However, the total conversion is only approximately 50% of wild type. (LOW ACTIVITY WITH IMPROVED RATIO; Table 2, Panels J-L).

TABLE 2
Effect of Single Mutations in CYP17

| CYB5A conc. | | | | |
|---|---|---|---|---|
| 0 | 0.2 | 0.4 | 0.8 | 1.5 |

A. B5WT − L102Q

| | | | | | |
|---|---|---|---|---|---|
| DHEA | 108% | 86% | 85% | 110% | 97% |
| 16A-steroid | 109% | 92% | 89% | 126% | 112% |
| 17OHP | 98% | 106% | 108% | 95% | 101% |
| 16A-str/DHEA ratio | 105% | 101% | 97% | 106% | 116% |
| Total conversion | 83% | 47% | 53% | 95% | 94% |

B. B5WT − D103S rep 1

| | | | | | |
|---|---|---|---|---|---|
| DHEA | 215% | 224% | 202% | 152% | 243% |
| 16A-steroid | 141% | 138% | 91% | 83% | 91% |
| 17OHP | 73% | 68% | 70% | 80% | 73% |
| 16A-str/DHEA ratio | 67% | 62% | 46% | 52% | 36% |
| Total conversion | 269% | 326% | 272% | 168% | 327% |

C. B5WT − D103S rep 2

| | | | | |
|---|---|---|---|---|
| DHEA | 116% | — | 108% | 115% |
| 16A-steroid | 81% | — | 74% | 81% |
| 17OHP | 92% | — | 95% | 91% |
| 16A-str/DHEA ratio | 71% | — | 69% | 71% |
| Total conversion | 131% | — | 120% | 146% |

D. B5WT − D103S rep 3

| | | | | | |
|---|---|---|---|---|---|
| DHEA | 105% | — | 103% | 107% | 102% |
| 16A-steroid | 74% | — | 101% | 76% | 84% |
| 17OHP | 99% | — | 99% | 97% | 100% |
| 16A-str/DHEA ratio | 71% | — | 98% | 70% | 82% |
| Total conversion | 132% | — | 138% | 140% | 137% |

E. B5WT − I104L

| | | | | |
|---|---|---|---|---|
| DHEA | 117% | — | 95% | 118% |
| 16A-steroid | 157% | — | 102% | 132% |
| 17OHP | 95% | — | 102% | 95% |
| 16A-str/DHEA ratio | 128% | — | 98% | 115% |
| Total conversion | 235% | — | 120% | 203% |

F. B5WT − S106A

| | | | | | |
|---|---|---|---|---|---|
| DHEA | 115% | 199% | 153% | 137% | 206% |
| 16A-steroid | 214% | 229% | 193% | 184% | 160% |
| 17OHP | 95% | 73% | 82% | 83% | 78% |
| 16A-str/DHEA ratio | 188% | 114% | 126% | 128% | 76% |
| Total conversion | 211% | 327% | 225% | 157% | 218% |

G. B5WT − S106D

| | | | | |
|---|---|---|---|---|
| DHEA | 12% | — | 25% | 13% |
| 16A-steroid | 46% | — | 10% | 16% |
| 17OHP | 122% | — | 124% | 124% |
| 16A-str/DHEA ratio | 444% | — | 32% | 43% |
| Total conversion | 90% | — | 50% | 58% |

H. B5WT − NQ108QG

| | | | | | |
|---|---|---|---|---|---|
| DHEA | 235% | 196% | 206% | 138% | 286% |
| 16A-steroid | 213% | 195% | 164% | 158% | 136% |
| 17OHP | 67% | 74% | 67% | 83% | 64% |
| 16A-str/DHEA ratio | 99% | 101% | 80% | 111% | 48% |
| Total conversion | 257% | 291% | 261% | 137% | 362% |

I. B5WT − I112V

| | | | | | |
|---|---|---|---|---|---|
| DHEA | 93% | 68% | 55% | 73% | 74% |
| 16A-steroid | 53% | 59% | 44% | 58% | 59% |
| 17OHP | 103% | 114% | 125% | 113% | 115% |
| 16A-str/DHEA ratio | 77% | 82% | 75% | 63% | 81% |
| Total conversion | 43% | 33% | 26% | 35% | 42% |

J. B5WT − L454V

| | | | | | |
|---|---|---|---|---|---|
| DHEA | 56% | — | 63% | 65% | 75% |
| 16A-steroid | 9% | — | 9% | 35% | 15% |
| 17OHP | 137% | — | 132% | 129% | 123% |
| 16A-str/DHEA ratio | 19% | — | 15% | 53% | 20% |
| Total conversion | 28% | — | 27% | 29% | 30% |

K. B5WT − L454V rep 2

| | | | | | |
|---|---|---|---|---|---|
| DHEA | 66% | — | — | — | 81% |
| 16A-steroid | 11% | — | — | — | 46% |
| 17OHP | 119% | — | — | — | 118% |
| 16A-str/DHEA ratio | 14% | — | — | — | 39% |
| Total conversion | 59% | — | — | — | 74% |

L. B5WT − L454V rep3

| | | | | |
|---|---|---|---|---|
| DHEA | 74% | — | — | 78% |
| 16A-steroid | 24% | — | — | 43% |
| 17OHP | 113% | — | — | 119% |
| 16A-str/DHEA ratio | 34% | — | — | 57% |
| Total conversion | 52% | — | — | 48% |

### Multiple Mutations

The double L102Q/I112V mutation improves the production of DHEA and 16A compared to the I112V mutant while maintaining the 20-30% improvement in the 16A/DHEA ratio of the I112V mutant. The total activity of the double mutants is also improved but it is still severely decreased compared to wild type CYP17. (LOW ACTIVITY; Table 3, Panels A and B). The L102Q/I112V/D103S triple mutant produces similar amounts of DHEA and more 16A than wild type CYP17, so the 16A/DHEA ratio is worse than for wild type CYP17. The overall activity is also dramatically lower than wild type CYP17. (LOW ACTIVITY; Table 3, panel C). The L102Q/D103S/I104L/NQ108QG/I112V mutant has much lower production of both DHEA and 16A with higher production of 17OHP than wild type CYP17. The 16A/DHEA ratio and overall activity are higher for this mutant than for wild type CYP17. (LOW OVERALL LYASE ACTIVITY; Table 3, Panel D).

TABLE 3
Effect of Multiple Mutations in CYP17

| CYB5A conc. | | | | |
|---|---|---|---|---|
| 0 | 0.2 | 0.4 | 0.8 | 1.5 |

A. L102Q/I112V

| | | | | | |
|---|---|---|---|---|---|
| DHEA | 103% | 91% | 86% | 111% | 100% |
| 16A-steroid | 119% | 68% | 73% | 95% | 66% |
| 17OHP | 99% | 105% | 108% | 95% | 102% |
| 16A-str/DHEA ratio | 117% | 69% | 81% | 79% | 71% |
| Total conversion | 58% | 46% | 36% | 58% | 47% |

B. L102Q/I112V rep 2

| | | | | |
|---|---|---|---|---|
| DHEA | 97% | — | 81% | 98% |
| 16A-steroid | 86% | — | 77% | 66% |
| 17OHP | 102% | — | 114% | 103% |
| 16A-str/DHEA ratio | 89% | — | 95% | 67% |
| Total conversion | 47% | — | 24% | 43% |

C. L102Q + I112V + D103S

| | | | | | |
|---|---|---|---|---|---|
| DHEA | 100% | 89% | 107% | 78% | 141% |
| 16A-steroid | 129% | 131% | 110% | 110% | 108% |
| 17OHP | 100% | 102% | 98% | 108% | 92% |
| 16A-str/DHEA ratio | 130% | 144% | 112% | 132% | 78% |
| Total conversion | 66% | 95% | 66% | 41% | 64% |

D. L102Q/D103S/I104L/NQ108QG/I112V

| | | | | |
|---|---|---|---|---|
| DHEA | 45% | — | 33% | 39% |
| 16A-steroid | 57% | — | 58% | 64% |
| 17OHP | 130% | — | 148% | 144% |
| 16A-str/DHEA ratio | 149% | — | 187% | 184% |
| Total conversion | 117% | — | 121% | 135% |

As summarized in Table 4, the D103S mutant has improved overall activity with no change in DHEAS production but 20% decrease in 16A production to reduce the 16A/DHEA ratio by 30%. The NQ108QG and S106A mutants have dramatically increased overall activity with a greater effect on DHEA production than 16A production with some improvement in the 16A/DHEA ratio. However, 16A production by these mutants is increased compared to wild type. The L454V mutant decreases production of DHEA by 25% and 16A by 55% to improve the 16A/DHEA ratio by 40%. However, the overall conversion rate is low.

TABLE 4
Summary of CYP17 mutations (most promising mutations are in bold)

| | | | | | |
|---|---|---|---|---|---|
| | | | | 16A/ | |
| CYP17 mutations with | | | | DHEA | Overall |
| wild type CYB5 | 17OHP | DHEA | 16A | ratio | conversion |

Single mutations

| | | | | | |
|---|---|---|---|---|---|
| L102Q | 1.012 | 0.967 | 1.116 | 1.162 | 0.941 |
| D103S | 0.913 | 1.146 | 0.806 | 0.706 | 1.455 |
| I104L | 0.948 | 1.180 | 1.325 | 1.154 | 2.034 |
| S106A | 0.784 | 2.059 | 1.601 | 0.757 | 2.179 |
| S106D | 1.242 | 0.126 | 0.164 | 0.434 | 0.576 |
| NQ108QG | 0.641 | 2.856 | 1.359 | 0.476 | 3.621 |
| I112V | 1.152 | 0.741 | 0.593 | 0.813 | 0.419 |
| N202T | — | — | 1.310 | — | .039 |
| IS344FN | — | 2.437 | .054 | — | .038 |
| N348S | — | — | — | — | .041 |
| L352M | — | — | — | — | .038 |
| L454V | 1.186 | 0.775 | 0.431 | 0.570 | 0.475 |

Combinations of mutations

| | | | | | |
|---|---|---|---|---|---|
| L102Q + I112V | 1.017 | 0.997 | 0.656 | 0.706 | 0.471 |
| L102Q + D103S + I112V | 0.920 | 1.414 | 1.075 | 0.777 | 0.637 |
| L102Q + D103S + | 1.444 | 0.388 | 0.640 | 1.842 | 1.349 |
| I104L + NQ108QG + | | | | | |
| I112V | | | | | |

Effect of Mutations in CYB5 with WT CYP17

### Single Mutations

The R52M mutant results in lower DHEA and 16A production, with improved 16A/DHEA ratio. There is similar overall activity with wild type CYB5 (DECREASED LYASE; Table 5, Panels A and B). The G57R mutant stimulates DHEA and 16A production similar to wild type CYB5 with small effects on the 16A/DHEA ratio. Overall activity is somewhat higher than wild type. (NO EFFECT; Table 5, Panels C and D). The N62S mutant has higher activity than wild type CYB5, with higher stimulation of DHEA than 16A synthesis with some improvement in the 16A/DHEA ratio. (INCREASED ACTIVITY WITH IMPROVED RATIO; Table 5, Panel E). The T70S mutant stimulates DHEA and 16A production similar to wild type CYB5 with no effect on the 16A/DHEA ratio and overall activity similar to wild type. (NO EFFECT; Table 5, Panels F and G). The N21K mutant produces less DHEA and 16A and more 17OHP than wild type with some improvement in the 16A/DHEA ratio. (DECREASED RATIO; Table 5, Panel H). The L28V mutant produces similar amounts of DHEA and less 16A than wild type with 25% improvement in the 16A/DHEA ratio. (IMPROVED RATIO; Table 5, Panel I).

TABLE 5
Effect of Single Mutations in CYB5

| CYB5A conc. | | | | |
|---|---|---|---|---|
| 0 | 0.2 | 0.4 | 0.8 | 1.5 |

A. R52M

| | | | | | |
|---|---|---|---|---|---|
| DHEA | 101% | 85% | 81% | 87% | 58% |
| 16A-steroid | 106% | 64% | 58% | 48% | 48% |
| 17OHP | 99% | 107% | 111% | 107% | 124% |
| 16A-str/DHEA ratio | 112% | 73% | 73% | 62% | 108% |
| Total conversion | 88% | 75% | 78% | 118% | 68% |

B. R52M rep 2

| | | | | | |
|---|---|---|---|---|---|
| DHEA | 102% | — | — | 82% | 76% |
| 16A-steroid | 108% | — | — | 63% | 49% |
| 17OHP | 98% | — | — | 117% | 128% |
| 16A-str/DHEA ratio | 105% | — | — | 77% | 65% |
| Total conversion | 110% | — | — | 102% | 94% |

C. G57R

| | | | | | |
|---|---|---|---|---|---|
| DHEA | 147% | 138% | 131% | 119% | 200% |
| 16A-steroid | 201% | 176% | 119% | 109% | 142% |
| 17OHP | 88% | 89% | 91% | 92% | 80% |
| 16A-str/DHEA ratio | 134% | 128% | 90% | 87% | 70% |
| Total conversion | 171% | 215% | 166% | 118% | 215% |

D. G57R rep2

| | | | | | |
|---|---|---|---|---|---|
| DHEA | 107% | — | 101% | 105% | 100% |
| 16A-steroid | 105% | — | 108% | 96% | 98% |
| 17OHP | 975 | — | 100% | 98% | 100% |
| 16A-str/DHEA ratio | 99% | — | 108% | 90% | 98% |
| Total conversion | 113% | — | 126% | 133% | 128% |

E. N62S

| | | | | | |
|---|---|---|---|---|---|
| DHEA | 125% | 174% | 159% | 142% | 246% |
| 16A-steroid | 104% | 148% | 109% | 98% | 126% |
| 17OHP | 94% | 80% | 82% | 84% | 72% |
| 16A-str/DHEA ratio | 81% | 82% | 74% | 71% | 56% |
| Total conversion | 127% | 241% | 192% | 133% | 275% |

F. T70S

| | | | | | |
|---|---|---|---|---|---|
| DHEA | 147% | 117% | 136% | 109% | 162% |
| 16A-steroid | 201% | 170% | 129% | 104% | 128% |
| 17OHP | 88% | 95% | 89% | 96% | 88% |
| 16A-str/DHEA ratio | 137% | 141% | 99% | 96% | 79% |
| Total conversion | 171% | 167% | 174% | 113% | 181% |

G. T70S rep2

| | | | | | |
|---|---|---|---|---|---|
| DHEA | 103% | — | 99% | 102% | 100% |
| 16A-steroid | 85% | — | 132% | 105% | 101% |
| 17OHP | 99% | — | 100% | 100% | 100% |
| 16A-str/DHEA ratio | 84% | — | 124% | 97% | 115% |
| Total conversion | 110% | — | 118% | 115% | 120% |

H. N21K

| | | | | | |
|---|---|---|---|---|---|
| DHEA | 98% | — | 84% | 82% | 74% |
| 16A-steroid | 105% | — | 80% | 58% | 54% |
| 17OHP | 101% | — | 110% | 113% | 122% |
| 16A-str/DHEA ratio | 107% | — | 96% | 70% | 72% |
| Total conversion | 92% | — | 95% | 108% | 124% |

I. L28V

| | | | | | |
|---|---|---|---|---|---|
| DHEA | 94% | — | 92% | 104% | 85% |
| 16A-steroid | 106% | — | 69% | 70% | 64% |
| 17OHP | 103% | — | 106% | 99% | 113% |
| 16A-str/DHEA ratio | 112% | — | 75% | 73% | 74% |
| Total conversion | 97% | — | 105% | 113% | 117% |

### Multiple Mutations

The R52M/N62S double mutant produces less DHEA and 16A with an improved 16A/DHEA ratio, but somewhat lower overall activity than wild type CYB5. (IMPROVED RATIO; Table 6, Panels A and B). There was decreased formation of both DHEA and 16A steroids by the R52M/G57R/N62S/T70S mutant of CYB5 with no effect on the ratio of 16A-steroids/DHEA or total conversion of pregnenolone. (NO EFFECT; Table 6, Panel C). There was some decrease in formation of both DHEA and 16A steroids by the N21K/L28V mutant of CYB5 with no effect on the ratio of 16A-steroids/DHEA or total conversion of pregnenolone. (NO EFFECT; Table 6, Panel D).

TABLE 6
Effect of Multiple Mutations in CYB5

| CYB5A conc. | | | | |
|---|---|---|---|---|
| 0 | 0.2 | 0.4 | 0.8 | 1.5 |

A. R52M/N62S

| | | | | | |
|---|---|---|---|---|---|
| DHEA | 86% | 74% | 50% | 53% | 86% |
| 16A-steroid | 120% | 64% | 49% | 45% | 38% |
| 17OHP | 103% | 107% | 116% | 120% | 104% |
| 16A-str/DHEA ratio | 146% | 87% | 102% | 76% | 52% |
| Total conversion | 64% | 95% | 68% | 49% | 96% |

B. R52M/N62S

| | | | | | |
|---|---|---|---|---|---|
| DHEA | 96% | — | — | 80% | 77% |
| 16A-steroid | 124% | — | — | 67% | 51% |
| 17OHP | 102% | — | — | 118% | 125% |
| 16A-str/DHEA ratio | 126% | — | — | 83% | 68% |
| Total conversion | 103% | — | — | 93% | 95% |

C. R52M/G57R/N62S/T70S

| | | | | |
|---|---|---|---|---|
| DHEA | 94% | — | 79% | 85% |
| 16A-steroid | 82% | — | 86% | 85% |
| 17OHP | 103% | — | 104% | 113% |
| 16A-str/DHEA ratio | 85% | — | 111% | 104% |
| Total conversion | 78% | — | 93% | 109% |

D. N21K/L28V − 17A1WT

| | | | | | |
|---|---|---|---|---|---|
| DHEA | 99% | — | 92% | 89% | 83% |
| 16A-steroid | 94% | — | 90% | 80% | 80% |
| 17OHP | 101% | — | 104% | 107% | 112% |
| 16A-str/DHEA ratio | 95% | — | 97% | 90% | 96% |
| Total conversion | 100% | — | 98% | 75% | 105% |

As summarized in Table 7, the R52M mutation has 25% decrease in overall activity, with about 40% decrease in DHEA production and 50% decrease in 16A production. N62S increases overall activity about 2 fold with a greater effect on DHEA than 16A to decrease the 16A/DHEA ratio by 45%. However, there is no net decrease in 16A production. The N21K and L28V mutations have no effect on overall conversion, but decrease 16A production by 35-45% and DHEA production by 15-25%. The combinations of R52M and N62S mutations decrease DHEA production by 15% and 16A production by 62% to reduce the 16A/DHEA ratio by 48%.

TABLE 7
Summary of CYB5 mutations (most promising mutations are in bold)

| | | | | | |
|---|---|---|---|---|---|
| | | | | 16A/ | |
| CYB5 mutations with | | | | DHEA | Overall |
| wild type CYP17 | 170HP | DHEA | 16A | ratio | conversion* |

Single mutations

| | | | | | |
|---|---|---|---|---|---|
| R52M | 1.242 | 0.578 | 0.478 | 1.081 | .765 |
| G57R | 1.002 | 0.999 | 0.979 | 0.979 | .865 |
| N62S | 0.716 | 2.468 | 1.262 | 0.557 | 2.163 |
| T70S | 1.000 | 1.000 | 1.007 | 1.149 | 1.095 |
| N21K | 1.215 | 0.743 | 0.539 | 0.715 | 1.358 |
| L28V | 1.129 | 0.853 | 0.638 | 0.743 | 1.201 |

Combinations of mutations

| | | | | | |
|---|---|---|---|---|---|
| R52M + N62S | 1.044 | 0.857 | 0.380 | 0.523 | 0.957 |
| R52M + G57R + | 1.126 | 0.852 | 0.854 | 1.043 | 1.402 |
| N62S + T70S | | | | | |
| N21K + L28V | 1.121 | 0.827 | 0.795 | 0.962 | 1.053 |

*Overall conversion expressed as ratio of (activity with b5/activity without b5) for each mutant. This measures the stimulatory effect of each b5 mutant with wild type CYP17.

### Combined Effects of Mutations in CYP17 and CYB5

The R52M-L102Q combination of mutants produces less 16A and DHEA, to possibly improve the 16A/DHEA ratio somewhat. However, the overall activity is lower than with wild type. (LOW ACTIVITY; Table 8, Panel A). The R52M-I112V combination of mutants produces less DHEA and 16A, to improve the 16A/DHEA ratio by 30-40%. However, the overall activity is dramatically lower than with wild type. (LOW OVERALL LYASE ACTIVITY; Table 8, Panel B). The R52M-L102Q/I112V combination of mutants produces lower levels of DHEA and dramatically less 16A than wild type, to improve the 16A/DHEA ratio by 55-60%. However, the overall activity is dramatically lower than with wild type (LOW ACTIVITY; Table 8, Panel C). The R52M mutant decreases maximum DHEA production by 25% and maximum 16A production by 54% with D103S CYP17 to improve the 16A/DHEA ratio by 40%. (IMPROVED RATIO; Table 8, Panel D). For the S106A mutant DHEA production was decreased by 47% and 16A production was decreased by 38% which made the 16A/DHEA ratio worse. There was also decreased overall activity (LOW ACTIVITY WITH POOR RATIO; Table 8, Panel E). For the NQ108QG mutant the decrease in DHEA was 14% while the decrease in 16A was 44% for 16A; this improves the ratio by 35%. There was also improved conversion by 25% over wild type. (IMPROVED RATIO AND ACTIVITY; Table 8, Panel F). The N62S/D103S combination has higher overall activity and decreased production of 16A while maintaining levels of DHEA similar to wild type CYP17. This results in an improvement in the 16A/DHEA ratio of 20-35% compared to wild type. (SOME IMPROVEMENT TO RATIO; Table 8, Panels G and H). The N62S/I104L combination has higher overall activity than wild type, with higher production of DHEA and 16A, with some increase in the 16A/DHEA ratio. (HIGH ACTIVITY BUT POOR RATIO; Table 8, Panel I). The N62S-S106D combination of mutants has much lower production of both DHEA and 16A with higher production of 17OHP than wild type CYP17. Overall activity is much lower than for wild type CYP17. (LOW ACTIVITY; Table 8, Panel J). The N62S-I112V/L102Q combination of mutants has somewhat decreased production of DHEA and 16A compared to wild type with some decrease in the 16A/DHEA ratio. The total activity of these mutants is severely decreased compared to wild type. (LOW ACTIVITY; Table 8, Panels K and L). The R52M/N62S mutant decreases maximum DHEA production by only 17% and maximum 16A production by 47% with D103S CYP17 to improve the 16A/DHEA ratio by 35%. (IMPROVED RATIO; Table 8, Panel M). For the S106A mutant DHEA production was decreased by 45% and 16A production was decreased by 20% which made the 16A/DHEA ratio worse. There was also decreased overall activity (LOW ACTIVITY WITH POOR RATIO; Table 8, Panel N). For the NQ108QG mutant the decrease in DHEA was 12% while the decrease in 16A was 23% for 16A; this has only marginal effects on the ratio. There was also improved conversion by 23% over wild type. (IMPROVED ACTIVITY, SMALL EFFECT ON RATIO; Table 8, Panel O). The R52M/N62S+L102Q/D103S/I112V combination of mutants produces less DHEA and 16A than wild type, with improved 16A/DHEA ratio of 50% or more. However, the overall activity is much lower than wild type. (LOW ACTIVITY; Table 8, Panels P and Q). The combined effects of these multiple mutations of CYB5 and CYP17 resulted in decreased formation of both DHEA and 16A steroids and higher formation of 17OHP compared to wild type. The 16A/DHEA ratio and overall conversion rate was higher than for wild type (DECREASED OVERALL LYASE; Table 8, Panel R). The G57R-D103S combination produces a small decrease in 16A while maintaining DHEA to produce only small effects on the ratio. Overall conversion is improved (HIGH ACTIVITY; Table 8, Panel S). The G57R-NQ108QG combination increased both 16A and DHEA with no effect on the ratio. Overall conversion is improved (HIGH ACTIVITY; Table 8, Panel T). The T70S-D103S combination had little effect on both 16A and DHEA with a small effect on the ratio. Overall conversion is improved (HIGH ACTIVITY; Table 8, Panel U). The T70S-NQ108QG combination increased both 16A and DHEA with no effect on the ratio. Overall conversion is improved (HIGH ACTIVITY; Table 8, Panel V). The N21K-D103S combination has 15% decrease in DHEA but 50% decrease in 16A to improve the ratio by 40% (INCREASED ACTIVITY WITH IMPROVED RATIO; Table 8, Panel W). The L28V-D103S combination has 10% decrease in DHEA and 35% decrease in 16A to improve the ratio by 30% (INCREASED ACTIVITY WITH IMPROVED RATIO; Table 8, Panel X). Finally, the N21K/L28V-D103S combination has 10% decrease in DHEA and 40% decrease in 16A to improve the ratio by 30% (IMPROVED RATIO; Table 8, Panel Y).

TABLE 8
Combined Effects of Mutations in CYP17 and CYB5

| CYB5A conc. | | | | |
|---|---|---|---|---|
| 0 | 0.2 | 0.4 | 0.8 | 1.5 |

A. R52M − L102Q

| | | | | | |
|---|---|---|---|---|---|
| DHEA | 103% | 82% | 75% | 87% | 70% |
| 16A-steroid | 82% | 73% | 65% | 61% | 61% |
| 17OHP | 99% | 108% | 114% | 107% | 117% |
| 16A-str/DHEA ratio | 86% | 99% | 86% | 74% | 103% |
| Total conversion | 73% | 58% | 51% | 82% | 74% |

B. R52M − I112V

| | | | | | |
|---|---|---|---|---|---|
| DHEA | 75% | 60% | 48% | 56% | 57% |
| 16A-steroid | 71% | 39% | 29% | 37% | 28% |
| 17OHP | 108% | 118% | 129% | 121% | 126% |
| 16A-str/DHEA ratio | 102% | 69% | 63% | 96% | 57% |
| Total conversion | 36% | 23% | 28% | 44% | 60% |

C. R52M − L102Q/I112V

| | | | | | |
|---|---|---|---|---|---|
| DHEA | 123% | 88% | 73% | 83% | 75% |
| 16A-steroid | 77% | 53% | 59% | 37% | 28% |
| 17OHP | 94% | 106% | 115% | 109% | 116% |
| 16A-str/DHEA ratio | 67% | 68% | 103% | 39% | 42% |
| Total conversion | 70% | 49% | 35% | 63% | 39% |

D. R52M − D103S

| | | | | | |
|---|---|---|---|---|---|
| DHEA | 103% | — | — | 80% | 76% |
| 16A-steroid | 83% | — | — | 54% | 46% |
| 17OHP | 99% | — | — | 119% | 128% |
| 16A-str/DHEA ratio | 80% | — | — | 68% | 60% |
| Total conversion | 112% | — | — | 110% | 108% |

E. R52M − S106A

| | | | | | |
|---|---|---|---|---|---|
| DHEA | 73% | — | — | 57% | 53% |
| 16A-steroid | 143% | — | — | 77% | 62% |
| 17OHP | 114% | — | — | 135% | 148% |
| 16A-str/DHEA ratio | 197% | — | — | 135% | 117% |
| Total conversion | 73% | — | — | 67% | 64% |

F. R52M − NQ108QG

| | | | | | |
|---|---|---|---|---|---|
| DHEA | 112% | — | — | 93% | 86% |
| 16A-steroid | 112% | — | — | 72% | 56% |
| 17OHP | 93% | — | — | 108% | 118% |
| 16A-str/DHEA ratio | 100% | — | — | 78% | 65% |
| Total conversion | 122% | — | — | 131% | 121% |

G. N62S − D103S

| | | | | |
|---|---|---|---|---|
| DHEA | 113% | — | 109% | 110% |
| 16A-steroid | 127% | — | 78% | 79% |
| 17OHP | 96% | — | 98% | 98% |
| 16A-str/DHEA ratio | 114% | — | 65% | 80% |
| Total conversion | 269% | — | 142% | 156% |

H. N62S − D103S rep 2-

| | | | | |
|---|---|---|---|---|
| DHEA | 110% | — | 108% | 117% |
| 16A-steroid | 79% | — | 83% | 91% |
| 17OHP | 95% | — | 95% | 90% |
| 16A-str/DHEA ratio | 73% | — | 78% | 79% |
| Total conversion | 127% | — | 118% | 146% |

I. N62S − I104L

| | | | | |
|---|---|---|---|---|
| DHEA | 104% | — | 123% | 132% |
| 16A-steroid | 151% | — | 119% | 176% |
| 17OHP | 98% | — | 93% | 90% |
| 16A-str/DHEA ratio | 152% | — | 94% | 148% |
| Total conversion | 212% | — | 167% | 236% |

J. N62S − S106D

| | | | | |
|---|---|---|---|---|
| DHEA | 18% | — | 14% | 7% |
| 16A-steroid | 51% | — | 25% | 40% |
| 17OHP | 120% | — | 127% | 125% |
| 16A-str/DHEA ratio | 303% | — | 186% | 204% |
| Total conversion | 105% | — | 40% | 40% |

K. N62S − I112V/L102Q

| | | | | |
|---|---|---|---|---|
| DHEA | 90% | — | 88% | 96% |
| 16A-steroid | 96% | — | 87% | 75% |
| 17OHP | 105% | — | 109% | 103% |
| 16A-str/DHEA ratio | 108% | — | 98% | 77% |
| Total conversion | 39% | — | 39% | 41% |

L. N62S − I112V/L102Q rep 2

| | | | | |
|---|---|---|---|---|
| DHEA | 80% | — | 103% | 80 |
| 16A-steroid | 34% | — | 38% | 43% |
| 17OHP | 106% | — | 101% | 106% |
| 16A-str/DHEA ratio | 36% | — | 33% | 54% |
| Total conversion | 48% | — | 68% | 39% |

M. R52M/N62S − D103S

| | | | | | |
|---|---|---|---|---|---|
| DHEA | 101% | — | — | 90% | 83% |
| 16A-steroid | 108% | — | — | 61% | 53% |
| 17OHP | 99% | — | — | 111% | 120% |
| 16A-str/DHEA ratio | 104% | — | — | 68% | 65% |
| Total conversion | 119% | — | — | 114% | 112% |

N. R52M/N62S − S106A

| | | | | | |
|---|---|---|---|---|---|
| DHEA | 65% | — | — | 58% | 55% |
| 16A-steroid | 162% | — | — | 82% | 80% |
| 17OHP | 120% | — | — | 135% | 144% |
| 16A-str/DHEA ratio | 240% | — | — | 143% | 146% |
| Total conversion | 75% | — | — | 65% | 65% |

O. R52M/N62S − NQ108QG

| | | | | | |
|---|---|---|---|---|---|
| DHEA | 110% | — | — | 93% | 88% |
| 16A-steroid | | — | — | 89% | 77% |
| 17OHP | 165% | — | — | | |
| 16A-str/DHEA ratio | 146% | — | — | 95% | 88% |
| Total conversion | 131% | — | — | 124% | 123% |

P. (R52M/N62S)CYB5 + (L102Q + I112V + D103S)
CYP17

| | | | | | |
|---|---|---|---|---|---|
| DHEA | 113% | 86% | 77% | 40% | 116% |
| 16A-steroid | 101% | 77% | 40% | 15% | 18% |
| 17OHP | 97% | 104% | 108% | 126% | 99% |
| 16A-str/DHEA ratio | 90% | 90% | 51% | 56% | 15% |
| Total conversion | 86% | 81% | 61% | 32% | 86% |

Q. (R52M/N62S)CYB5 + (L102Q + I112V + D103S)
CYP17 rep 2

| | | | | | |
|---|---|---|---|---|---|
| DHEA | 98% | — | 90% | 82% | 84% |
| 16A-steroid | 42% | — | 51% | 39% | 33% |
| 17OHP | 102% | — | 106% | 111% | 113% |
| 16A-str/DHEA ratio | 44% | — | 56% | 48% | 43% |
| Total conversion | 44% | — | 42% | 40% | 39% |

R. R52M/G57R/N62S/T70S − L102Q/D103S/I104L/
NQ108QG/I112V

| | | | | |
|---|---|---|---|---|
| DHEA | 36% | — | 25% | 26% |
| 16A-steroid | 62% | — | 41% | 50% |
| 17OHP | 135% | — | 154% | 154% |
| 16A-str/DHEA ratio | 171% | — | 166% | 198% |
| Total conversion | 128% | — | 110% | 132% |

S. G57R − D103S

| | | | | | |
|---|---|---|---|---|---|
| DHEA | 104% | — | 102% | 111% | 109% |
| 16A-steroid | 80% | — | 91% | 77% | 91% |
| 17OHP | 99% | — | 99% | 95% | 95% |
| 16A-str/DHEA ratio | 77% | — | 89% | 70% | 84% |
| Total conversion | 140% | — | 146% | 172% | 167% |

T. G57R − NQ108QG

| | | | | | |
|---|---|---|---|---|---|
| DHEA | 118% | — | 126% | 128% | 126% |
| 16A-steroid | 155% | — | 147% | 118% | 123% |
| 17OHP | 92% | — | 87% | 85% | 83% |
| 16A-str/DHEA ratio | 130% | — | 117% | 93% | 98% |
| Total conversion | 196% | — | 252% | 262% | 252% |

U. T70S − D103S

| | | | | | |
|---|---|---|---|---|---|
| DHEA | 111% | — | 106% | 113% | 109% |
| 16A-steroid | 88% | — | 103% | 93% | 94% |
| 17OHP | 96% | — | 97% | 93% | 95% |
| 16A-str/DHEA ratio | 79% | — | 97% | 82% | 86% |
| Total conversion | 157% | — | 165% | 170% | 171% |

V. T70S − NQ108QG

| | | | | | |
|---|---|---|---|---|---|
| DHEA | 114% | — | 111% | 119% | 122% |
| 16A-steroid | 169% | — | 154% | 103% | 120% |
| 17OHP | 94% | — | 94% | 90% | 86% |
| 16A-str/DHEA ratio | 141% | — | 127% | 87% | 118% |
| Total conversion | 195% | — | 200% | 228% | 229% |

W. N21K − D103S

| | | | | | |
|---|---|---|---|---|---|
| DHEA | 103% | — | 91% | 86% | 84% |
| 16A-steroid | 80% | — | 78% | 77% | 49% |
| 17OHP | 100% | — | 105% | 108% | 113% |
| 16A-str/DHEA ratio | 78% | — | 86% | 89% | 59% |
| Total conversion | 124% | — | 141% | 141% | 155% |

X. L28V − D103S

| | | | | | |
|---|---|---|---|---|---|
| DHEA | 101% | — | 95% | 89% | 92% |
| 16A-steroid | 76% | — | 77% | 89% | 64% |
| 17OHP | 101% | — | 103% | 107% | 107% |
| 16A-str/DHEA ratio | 75% | — | 80% | 100% | 69% |
| Total conversion | 140% | — | 148% | 143% | 165% |

Y. N21K/L28V − D103S

| | | | | | |
|---|---|---|---|---|---|
| DHEA | 97% | — | 99% | 90% | 87% |
| 16A-steroid | 79% | — | 63% | 58% | 59% |
| 17OHP | 102% | — | 102% | 108% | 111% |
| 16A-str/DHEA ratio | 81% | — | 64% | 65% | 68% |
| Total conversion | 110% | — | 113% | 87% | 124% |

As summarized in Table 9, the R52M/N62S combination with the triple L102Q/D103S/I112V mutant has the best 16A/DHEA ratio, but reduced overall conversion. This double CYB5 mutant was no different from the R52M mutant with CYP17 mutants D103S, S106A and NQ108QG that have increased activity. The best combinations to date are the N21K, L28V and R52M mutants of CYB5 with the D103S mutant of CYP17 and the R52M mutants of CYB5 with the NQ108QG mutant of CYP17.

TABLE 9
Summary of Combinations of CYB5 and CYP17 Mutations (most
promising combinations are in bold)

| | | | | 16A/ | |
|---|---|---|---|---|---|
| CYB5 mutations with | | | | DHEA | Overall |
| CYP17 mutations | 17OHP | DHEA | 16A | ratio | conversion |
| R52M + L102Q | 1.174 | 0.699 | 0.607 | 1.032 | 0.735 |
| R52M + I112V | 1.257 | 0.566 | 0.282 | 0.567 | 0.600 |
| R52M + L102Q/I112V | 1.500 | 1.162 | 0.750 | 0.282 | 0.418 |
| R52M/D103S | 1.282 | 0.761 | 0.457 | 0.600 | 1.075 |
| R52M/S106A | 1.484 | 0.529 | 0.616 | 1.167 | 0.638 |
| R52M/NQ108QG | 1.176 | 0.861 | 0.563 | 0.653 | 1.212 |
| N62S + D103S | 0.897 | 1.166 | 0.912 | 0.787 | 1.457 |
| N62S + 104L | 0.904 | 1.317 | 1.760 | 1.484 | 2.363 |
| N62S + S106D | 1.252 | 0.071 | 0.399 | 2.042 | 0.437 |
| N62S + L102Q/I112V | 1.032 | 0.963 | 0.748 | 0.765 | 0.415 |
| R52M + N62S/D103S | 1.195 | 0.827 | 0.534 | 0.645 | 1.123 |
| R52M + N62S/S106A | 1.437 | 0.546 | 0.799 | 1.462 | 0.654 |
| R52M + N62S/NQ108QG | 1.130 | 0.877 | 0.771 | 0.881 | 1.227 |
| R52M + N62S + | 1.130 | 0.839 | 0.333 | 0.426 | 0.385 |
| L102Q/D103S/I112V | | | | | |
| R52M/G57R/N62S/ | 1.536 | 0.257 | 0.503 | 1.979 | 1.536 |
| T70S + L102Q/D103S/ | | | | | |
| I104L/NQ108QG/I112V | | | | | |
| G57R + D103S | 0.950 | 1.085 | 0.905 | 0.836 | 1.672 |
| G57R + NQ108QG | 0.833 | 1.255 | 1.231 | 0.983 | 2.521 |
| T70S + D103S | 0.947 | 1.087 | 0.937 | 0.863 | 1.713 |
| T70S + NQ108QG | 0.855 | 1.221 | 1.201 | 1.180 | 2.286 |
| N21K + D103S | 1.132 | 0.835 | 0.490 | 0.585 | 1.551 |
| L28V + D103S | 1.068 | 0.924 | 0.643 | 0.693 | 1.652 |
| N21K/L28V + D103S | 1.110 | 0.867 | 0.588 | 0.677 | 1.240 |

### Discussion

CYP17 Mutants with Wild Type CYB5

The D103S mutant was best with decreased 16A/DHEA ratio and increased overall activity. The NQ108QG mutant had dramatically increased activity with some improvement in 16A/DHEA ratio. The L454V mutant had the greatest decrease in 16A/DHEA ratio, but decreased overall activity. The combination of D103S and L454V is now being investigated. The N202T, IS344FN, N348S, and L352M mutants did not express well and are also under investigation.

CYB5 Mutants with Wild Type CYP17

The R52M mutant of CYB5 decreased production of both 16A and DHEA to the same extent, with no effect on the 16A/DHEA ratio and decreased overall activity. The N62S mutant increased DHEA production more than 16A production for an increase in overall activity with a decreased 16A/DHEA ratio. The combination of R52M and N62S decreased production of DHEA more than 16A for a decrease in 16A/DHEA ratio with similar activity to wild type CYB5. The N21K and L28V mutants also decreased 16A more than DHEA for a decreased ratio with good activity.

### Combination of CYP17 and CYB5 Mutants

The most effective combinations were CYP17 D103S with either CYB5 mutants N21K, L28V, N21K+L28V, R52M or R52M+N62S, which improve the 16A/DHEA ratio by 35-40%. This is only marginally better than D103S with wild type CYB5 and similar to the R52M+N62S mutant of CYB5 with wild type CYP17. Thus far it seems that the effects of mutants in CYB5 and CYP17 are not additive. The most direct approach is the R52M+N62S mutant of CYB5 with wild type CYP17.

### LITERATURE REFERENCES

Abe K, Sugita Y (1979) Properties of cytochrome b5 and methemoglobin reduction in human erythrocytes Eur. J. Biochem. 101: 423-428.
Auchus R J and Miller W L (1999). Molecular modeling of human P450c17 (17α-hydroxylase/17,20 lyase): Insights into reaction mechanisms and effects of mutations. Mol. Endo. 13:1169-1182.
Banci L, Bertini I, Ferroni F and Rosato A. (1997). Solution structure of reduced microsomal rat cytochrome b5. Eur. J. Biochem. 249:270-279.
Billen M J and Squires E J (2009). The role of porcine cytochrome b5A and cytochrome b5B in the regulation of cytochrome 45017A1 activities. J. Steroid Biochem. Mol. Biol. 113:98-104.
Bonneau M, Meadus W J, Squires E J (1992) Effects of exogenous porcine somatotropin on performance, testicular steroid production and fat levels of boar-taint-related compounds in young boars Can. J. Anim. Sci. 72: 537-545.
Claus R, Hoffman B, Karg H (1971) Determination of 5-androst-16-ene-3-one, a boar taint steroid in pigs, with reference to relationship to testosterone J. Anim. Sci. 33:1293-1297.
Cooke, G M and Gower D B (1977). The submicrosomal distribution in rat and boar testis of some enzymes involved in androgen and 16-androstene biosynthesis. Biochim Biophys Acta 498: 265-271.
DeVore N M and Scott E E (2012) Structures of cytochrome P450 17A1 with prostate cancer drugs abiraterone and TOK-001. Nature 482: 116-120.
Dufort I, Rheault P, Huang X F, Soucy P, Luu-The V (1999) Characteristics of a highly labile human type 5 17β-hydroxysteroid dehydrogenase Endocrinol. 140: 568-574.
Gower D B (1972) 16-Unsaturated C19 steroids: a review of their chemistry, biochemistry and possible physiological role J. Steroid Biochem. 3: 45-103.
Gunn M, Allen P, Bonneau M, Byrne D V, Cinotti S, Fredriksen B, Hansen L L, Karlsson A H, Linder M G, Lundstrom K, Moerton D B, Prunier A, Squires J, Tuyttens F, Velarde A, von Borell E H, Wood J. (2004) Welfare aspects of the castration of piglets Scientific report of the Scientific Panel for Animal Health and Welfare, The European Food Safety Authority Journal 91, 1-18.
Ilan Z, Ilan R, Cinit D I (1981) Evidence for a new physiological role of hepatic NADPH: Ferricytochrome (P-450) Oxidoreductase J. Biol. Chem. 256: 10066-10072.
Lee-Robichaud P, Akhtar, M E, Wright, J N, Sheikh, Q I and Akhtar, M. (2004). The cationic charges on arg 347, arg358 and arg449 of human cytochrome P450c17 (CYP17) are essential for the enzyme's cytochrome b5-dependent acyl-carbon cleavage activities. J. Steroid Biochem. Mol. Biol. 92:119-130.
Lu A Y H, West S B (1978) Reconstituted mammalian mixed-function oxidases: requirements, specificities and other properties Pharmacol. Ther. Part A. 2: 337-359.
Luu-The V, Zhang L, Poirier D, Labrie F (2005) Characteristics of human types 1, 2 and 3 17β-hydroxysteroid dehydrogenase activities: oxidation/reduction and inhibition J. Steroid Biochem. Mol. Biol. 96: 217-228.
McLaughlin, L A, Ronseaux, S, Finn, R D, Henderson, C L and Wolf, C R (2010). Deletion of microsomal cytochrome b5 profoundly affects hepatic and extrahepatic drug metabolism. Mol. Pharmacol. 78:269-278.
Meadus W J, Mason J I, Squires E J (1993) Cytochrome P450c17 from porcine and bovine adrenal catalyses the formation of 5,16-androstadien-3β-ol from pregnenolone in the presence of cytochrome b5. J. Steroid Biochem. Mol. Biol. 46: 565-572.
Naffin-Olivod J L and Auchus R J (2006). Human cytochrome b5 requires residues E48 and E49 to stimulate the 17,20 lyase activity of cytochrome P450c17. Biochemistry 45:755-762.
Ogishima T, Kinoshita J, Mitani F, Suematsu M, Ito A (2003) Identification of outer mitochondrial membrane cytochrome b5 as a modulator for androgen synthesis in leydig cells J. Biol. Chem. 278: 21204-21211.
Ozols J (1976) The role of microsomal cytochrome b5 in the metabolism of ethanol, drugs and the desaturation of fatty acids Ann. Clin. Res. 8: 182-192.
Pandley A V and Miller W L (2005). Regulation of 17,20 lyase activity by cytochrome b5 and by serine phosphorylation of P450c17. J. Biol Chem. 280:13265-13271
Peacock J, Lou Y, Lundström K, Squires E J. (2008). The effect of a c.-8G>T polymorphism on the expression of cytochrome b5A and boar taint in pigs. Anim Genet. 2008 39:15-21.
Sinclair P A, Squires E J, Raeside J I, Renaud R (1995) Synthesis of free and sulphoconjugated 16-androstene steroids by the Leydig cells of the mature domestic boar J. Steroid Biochem. Mol. Biol. 55: 581-587.
Swart A C, Storbeck, K-H and Swart, P (2010). A single amino acid residue, Ala 105, confers 16α-hydroxylase activity to human cytochrome P450 17α-hydroxylase/17,20 lyase. J. steroid Biochem. Mol. Biol. 119:112-120.
Wang T-H, Tee M K and Miller W L (2010). Human cytochrome P450c17: single step purification and phosphorylation of serine 258 by protein kinase A. Endocrinology 151:1677-1684.
Yamazaki T, Ohno T, Sakaki T, Akiyoshi-Shibata M, Yabusaki Y, Imai T, Kominami S (1998) Kinetic analysis of successive reactions catalyzed by bovine cytochrome P45017α, lyase Biochem. 37: 2800-2806.
Yoshida T, Kikuchi G (1978) Features of the reaction of heme degradation catalyzed by the reconstituted microsomal heme oxygenase system J. Biol. Chem. 253: 4230-4236.

### Example 2

Sus scrofa cytochrome b5 type A (Pig CYB5A)
(SEQ ID NO: 1)
ATG GCCGAACAGT CCGACAAAGC CGTGAAGTAT TACACCCTGG
AAGAGATCCA GAAGCACAAC AACAGCAAGA GCACCTGGCT
AATCCTGCAC CACAAAGTGTACGATTTGAC CAAATTTTTG
GAGGAGCATC CTGGTGGGGA AGAAGTCTTA
AGGGAACAAGCTGGAGGTGA TGCTACTGAA AATTTTGAGG
ATGTTGGACA CTCCACAGAT GCTCGAGAGTTGTCCAAAAC
GTTCATCATT GGGGAGCTGC ATCCGGATGA CAGATCAAAG
ATTGCCAAGCCTTCGGAAAC TCTTATTACC ACTGTTGAAT
CTAATTCCAG CTGGTGGACC AACTGGGTGA TCCCAGCCAT
CTCAGCACTG GTTGTATCCC TGATGTATCA CTTCTACACA
TCGGAAAACTAA
Sus scrofa cytochrome b5 type A (Pig CYB5A)
(SEQ ID NO: 2)
MAEQSDKAVKYYTLEEIQKHNNSKSTWLILHHKVYDLTKFLEEHPGGEE
VLREQAGGDATENFEDVGHSTDARELSKTFIIGELHPDDRSKIAKPSET
LITTVESNSSWWTNWVIPAISALVVSLMYHFYTSEN STOP
Sus scrofa cytochrome P450 17A1 (Pig CYP17A1)
(SEQ ID NO: 3)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTGTCAGACAACCAAAAGGGGATTGCCTTCG
CCGACCATGGTACCTCCTGGCAGCTGCATCGGAAGCTGGCACTGAGCAC
CTTTTCCCTGTTCAAGGGTGGCAACCTGAAGCTGGAGAACATCATTAAT
CAAGAAATCAAAGTACTGTGCGATTTCCTGGCCACACGGAATGGAGAGT
CCATTGATTTGGCCCAGCCTCTCTCTCTGGCGATGACCAACATAGTCAG
CTTTATCTGCTTCAACTTCTCCTTCAAGAAGGGGGATCCCGCGCTGCAG
GCCATAGTGAATTTCAATGATGGCATCCTGGATGCTGTGGGCAAGGAAA
TTTTGTATGACATGTTCCCTGGAATTAGGATTTTACCCAGCCAAACTCT
GGAAAACATGAAGCAGTGTGTTAGAATGCGAAACGAATTGCTGCGGGAA
ATCCTTGAAAACCGTAAGGAGAACTACAGCAGAAACTCCATCACTAACT
TGTTGGACATAATGATCCAAGCCAAGACGAACGCAGAAAGTAACACTGG
TGGCCCAGACCACAATTTAAAGCTGCTTTCAGACAGACACATGCTCGCC
ACTGTTGCGGACATCTTTGGGGCCGGTGTGGAGACTTCTGCCTCTGTGG
TAAAGTGGATCGTGGCCTTCCTGCTACACTATCCTCTGCTGAGGAAGAA
GATCCAGGATGCTATCGACCAGAATATTGGTTTCAATCGTGCCCCATCT
ATCAGCGACCGGAACCAACTTGTCCTCCTGGAGGCCACCATCCGAGAGG
TGCTTCGATTCCGGCCTGTGTCCCCTACGCTCATCCCCCACAGGGCTAT
CATTGACTCCAGCATTGGCGAATTTACCATTGACAAGGACACAGATGTC
GTCGTCAATCTGTGGGCACTGCATCACAATGAGAAGGAGTGGCTCCGGC
CCGACCTGTTCATGCCTGAGCGCTTCCTGGACCCCACGGGAACCCAGCT
CATCTCACCATCATTGAGCTACTTGCCCTTCGGAGCAGGACCCCGCTCT
TGCGTAGGGGAGATGCTAGCCCGCCAGGAGCTCTTCCTCTTCACGGCTG
GATTGCTGCAGAGGTTCGACCTGGAGCTCCCAGATGATGGGCAGCTACC
CTGTCTCGTGGGCAACCCCAGTTTGGTCCTGCAGATAGATCCTTTCAAA
GTGAAGATCAAGGAGCGCCAGGCCTGGAAGGAAGCCCACACTGAGGGGA
GTACCTCCTGA
Sus scrofa cytochrome P450 17A1 (Pig CYP17A1)
(SEQ ID NO: 4)
MWVLLVFFLLTLTYLFWPKTKGSGAKYPRSLPVLPVVGSLPFLPRRGHQ
HMNFFKLQDKYGPIFSFRLGSKTTVVIGDHQLAKEVLLKKGKEFSGRPR
VMTLDILSDNQKGIAFADHGTSWQLHRKLALSTFSLFKGGNLKLENIIN
QEIKVLCDFLATRNGESIDLAQPLSLAMTNIVSFICFNFSFKKGDPALQ
AIVNFNDGILDAVGKEILYDMFPGIRILPSQTLENMKQCVRMRNELLRE
ILENRKENYSRNSITNLLDIMIQAKTNAESNTGGPDHNLKLLSDRHMLA
TVADIFGAGVETSASVVKWIVAFLLHYPLLRKKIQDAIDQNIGFNRAPS
ISDRNQLVLLEATIREVLRFRPVSPTLIPHRAIIDSSIGEFTIDKDTDV
VVNLWALHHNEKEWLRPDLFMPERFLDPTGTQLISPSLSYLPFGAGPRS
CVGEMLARQELFLFTAGLLQRFDLELPDDGQLPCLVGNPSLVLQIDPFK
VKIKERQAWKEAHTEGSTS Stop
CYB5A-N21K
(SEQ ID NO: 23)
ATGGCCGAACAGTCCGACAAAGCCGTGAAGTATTACACCCTGGAAGAGA
TCCAGAAGCACAA[G]AACAGCAAGAGCACCTGGCTAATCCTGCACCAC
AAAGTGTACGATTTGACCAAATTTTTGGAGGAGCATCCTGGTGGGGAAG
AAGTCTTAAGGGAACAAGCTGGAGGTGATGCTACTGAAAATTTTGAGGA
TGTTGGACACTCCACAGATGCTCGAGAGTTGTCCAAAACGTTCATCATT
GGGGAGCTGCATCCGGATGACAGATCAAAGATTGCCAAGCCTTCGGAAA
CTCTTATTACCACTGTTGAATCTAATTCCAGCTGGTGGACCAACTGGGT
GATCCCAGCCATCTCAGCACTGGTTGTATCCCTGATGTATCACTTCTAC
ACATCGGAAAACTAA
CYB5A-L28V N21K
(SEQ ID NO: 24)
ATGGCCGAACAGTCCGACAAAGCCGTGAAGTATTACACCCTGGAAGAGA
TCCAGAAGCACAACAACAGCAAGAGCACCTGG[G]TAATCCTGCACCAC
AAAGTGTACGATTTGACCAAATTTTTGGAGGAGCATCCTGGTGGGGAAG
AAGTCTTAAGGGAACAAGCTGGAGGTGATGCTACTGAAAATTTTGAGGA
TGTTGGACACTCCACAGATGCTCGAGAGTTGTCCAAAACGTTCATCATT
GGGGAGCTGCATCCGGATGACAGATCAAAGATTGCCAAGCCTTCGGAAA
CTCTTATTACCACTGTTGAATCTAATTCCAGCTGGTGGACCAACTGGGT
GATCCCAGCCATCTCAGCACTGGTTGTATCCCTGATGTATCACTTCTAC
ACATCGGAAAACTAA
CYB5A-R52M
(SEQ ID NO: 25)
ATGGCCGAACAGTCCGACAAAGCCGTGAAGTATTACACCCTGGAAGAGA
TCCAGAAGCACAACAACAGCAAGAGCACCTGGCTAATCCTGCACCACAA
AGTGTACGATTTGACCAAATTTTTGGAGGAGCATCCTGGTGGGGAAGAA
GTCTTAA[T]GGAACAAGCTGGAGGTGATGCTACTGAAAATTTTGAGGA
TGTTGGACACTCCACAGATGCTCGAGAGTTGTCCAAAACGTTCATCATT
GGGGAGCTGCATCCGGATGACAGATCAAAGATTGCCAAGCCTTCGGAAA
CTCTTATTACCACTGTTGAATCTAATTCCAGCTGGTGGACCAACTGGGT
GATCCCAGCCATCTCAGCACTGGTTGTATCCCTGATGTATCACTTCTAC
ACATCGGAAAACTAA
CYB5A-G57R
(SEQ ID NO: 26)
ATGGCCGAACAGTCCGACAAAGCCGTGAAGTATTACACCCTGGAAGAGA
TCCAGAAGCACAACAACAGCAAGAGCACCTGGCTAATCCTGCACCACAA
AGTGTACGATTTGACCAAATTTTTGGAGGAGCATCCTGGTGGGGAAGAA
GTCTTAAGGGAACAAGCTGGA[C]GTGATGCTACTGAAAATTTTGAGGA
TGTTGGACACTCCACAGATGCTCGAGAGTTGTCCAAAACGTTCATCATT
GGGGAGCTGCATCCGGATGACAGATCAAAGATTGCCAAGCCTTCGGAAA
CTCTTATTACCACTGTTGAATCTAATTCCAGCTGGTGGACCAACTGGGT
GATCCCAGCCATCTCAGCACTGGTTGTATCCCTGATGTATCACTTCTAC
ACATCGGAAAACTAA
CYB5A-N62S
(SEQ ID NO: 27)
ATGGCCGAACAGTCCGACAAAGCCGTGAAGTATTACACCCTGGAAGAGA
TCCAGAAGCACAACAACAGCAAGAGCACCTGGCTAATCCTGCACCACAA
AGTGTACGATTTGACCAAATTTTTGGAGGAGCATCCTGGTGGGGAAGAA
GTCTTAAGGGAACAAGCTGGAGGTGATGCTACTGAAA[G]TTTTGAGGA
TGTTGGACACTCCACAGATGCTCGAGAGTTGTCCAAAACGTTCATCATT
GGGGAGCTGCATCCGGATGACAGATCAAAGATTGCCAAGCCTTCGGAAA
CTCTTATTACCACTGTTGAATCTAATTCCAGCTGGTGGACCAACTGGGT
GATCCCAGCCATCTCAGCACTGGTTGTATCCCTGATGTATCACTTCTAC
ACATCGGAAAACTAA
CYB5A-T70S
(SEQ ID NO: 28)
ATGGCCGAACAGTCCGACAAAGCCGTGAAGTATTACACCCTGGAAGAGA
TCCAGAAGCACAACAACAGCAAGAGCACCTGGCTAATCCTGCACCACAA
AGTGTACGATTTGACCAAATTTTTGGAGGAGCATCCTGGTGGGGAAGAA
GTCTTAAGGGAACAAGCTGGAGGTGATGCTACTGAAAATTTTGAGGATG
TTGGACACTCC[T]CAGATGCTCGAGAGTTGTCCAAAACGTTCATCATT
GGGGAGCTGCATCCGGATGACAGATCAAAGATTGCCAAGCCTTCGGAAA
CTCTTATTACCACTGTTGAATCTAATTCCAGCTGGTGGACCAACTGGGT
GATCCCAGCCATCTCAGCACTGGTTGTATCCCTGATGTATCACTTCTAC
ACATCGGAAAACTAA
CYB5A-N21K + L28V
(SEQ ID NO: 29)
ATGGCCGAACAGTCCGACAAAGCCGTGAAGTATTACACCCTGGAAGAGA
TCCAGAAGCACAA[G]AACAGCAAGAGCACCTGG[G]TAATCCTGCACC
ACAAAGTGTACGATTTGACCAAATTTTTGGAGGAGCATCCTGGTGGGGA
AGAAGTCTTAAGGGAACAAGCTGGAGGTGATGCTACTGAAAATTTTGAG
GATGTTGGACACTCCACAGATGCTCGAGAGTTGTCCAAAACGTTCATCA
TTGGGGAGCTGCATCCGGATGACAGATCAAAGATTGCCAAGCCTTCGGA
AACTCTTATTACCACTGTTGAATCTAATTCCAGCTGGTGGACCAACTGG
GTGATCCCAGCCATCTCAGCACTGGTTGTATCCCTGATGTATCACTTCT
ACACATCGGAAAACTAA
CYB5A-R52M + N62S
(SEQ ID NO: 30)
ATGGCCGAACAGTCCGACAAAGCCGTGAAGTATTACACCCTGGAAGAGA
TCCAGAAGCACAACAACAGCAAGAGCACCTGGCTAATCCTGCACCACAA
AGTGTACGATTTGACCAAATTTTTGGAGGAGCATCCTGGTGGGGAAGAA
GTCTTAA[T]GGAACAAGCTGGAGGTGATGCTACTGAAA[G]TTTTGAG
GATGTTGGACACTCCACAGATGCTCGAGAGTTGTCCAAAACGTTCATCA
TTGGGGAGCTGCATCCGGATGACAGATCAAAGATTGCCAAGCCTTCGGA
AACTCTTATTACCACTGTTGAATCTAATTCCAGCTGGTGGACCAACTGG
GTGATCCCAGCCATCTCAGCACTGGTTGTATCCCTGATGTATCACTTCT
ACACATCGGAAAACTAA
CYB5A-QM R52M/G57R/N62S/T70S
(SEQ ID NO: 31)
ATGGCCGAACAGTCCGACAAAGCCGTGAAGTATTACACCCTGGAAGAGA
TCCAGAAGCACAACAACAGCAAGAGCACCTGGCTAATCCTGCACCACAA
AGTGTACGATTTGACCAAATTTTTGGAGGAGCATCCTGGTGGGGAAGAA
GTCTTAA[T]GGAACAAGCTGGA[C]GTGATGCTACTGAAA[G]TTTTG
AGGATGTTGGACACTCC[T]CAGATGCTCGAGAGTTGTCCAAAACGTTC
ATCATTGGGGAGCTGCATCCGGATGACAGATCAAAGATTGCCAAGCCTT
CGGAAACTCTTATTACCACTGTTGAATCTAATTCCAGCTGGTGGACCAA
CTGGGTGATCCCAGCCATCTCAGCACTGGTTGTATCCCTGATGTATCAC
TTCTACACATCGGAAAACTAA
CYP17A1-L102Q
(SEQ ID NO: 32)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTC[A]AGACATCCTGTCAGACAACCAAAAGGGGATTGCCTT
CGCCGACCATGGTACCTCCTGGCAGCTGCATCGGAAGCTGGCACTGAGC
ACCTTTTCCCTGTTCAAGGGTGGCAACCTGAAGCTGGAGAACATCATTA
ATCAAGAAATCAAAGTACTGTGCGATTTCCTGGCCACACGGAATGGAGA
GTCCATTGATTTGGCCCAGCCTCTCTCTCTGGCGATGACCAACATAGTC
AGCTTTATCTGCTTCAACTTCTCCTTCAAGAAGGGGGATCCCGCGCTGC
AGGCCATAGTGAATTTCAATGATGGCATCCTGGATGCTGTGGGCAAGGA
AATTTTGTATGACATGTTCCCTGGAATTAGGATTTTACCCAGCCAAACT
CTGGAAAACATGAAGCAGTGTGTTAGAATGCGAAACGAATTGCTGCGGG
AAATCCTTGAAAACCGTAAGGAGAACTACAGCAGAAACTCCATCACTAA
CTTGTTGGACATAATGATCCAAGCCAAGACGAACGCAGAAAGTAACACT
GGTGGCCCAGACCACAATTTAAAGCTGCTTTCAGACAGACACATGCTCG
CCACTGTTGCGGACATCTTTGGGGCCGGTGTGGAGACTTCTGCCTCTGT
GGTAAAGTGGATCGTGGCCTTCCTGCTACACTATCCTCTGCTGAGGAAG
AAGATCCAGGATGCTATCGACCAGAATATTGGTTTCAATCGTGCCCCAT
CTATCAGCGACCGGAACCAACTTGTCCTCCTGGAGGCCACCATCCGAGA
GGTGCTTCGATTCCGGCCTGTGTCCCCTACGCTCATCCCCCACAGGGCT
ATCATTGACTCCAGCATTGGCGAATTTACCATTGACAAGGACACAGATG
TCGTCGTCAATCTGTGGGCACTGCATCACAATGAGAAGGAGTGGCTCCG
GCCCGACCTGTTCATGCCTGAGCGCTTCCTGGACCCCACGGGAACCCAG
CTCATCTCACCATCATTGAGCTACTTGCCCTTCGGAGCAGGACCCCGCT
CTTGCGTAGGGGAGATGCTAGCCCGCCAGGAGCTCTTCCTCTTCACGGC
TGGATTGCTGCAGAGGTTCGACCTGGAGCTCCCAGATGATGGGCAGCTA
CCCTGTCTCGTGGGCAACCCCAGTTTGGTCCTGCAGATAGATCCTTTCA
AAGTGAAGATCAAGGAGCGCCAGGCCTGGAAGGAAGCCCACACTGAGGG
GAGTACCTCCTGA
CYP17A1-D103S
(SEQ ID NO: 33)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTA[TC]CATCCTGTCAGACAACCAAAAGGGGATTGCCTT
CGCCGACCATGGTACCTCCTGGCAGCTGCATCGGAAGCTGGCACTGAGC
ACCTTTTCCCTGTTCAAGGGTGGCAACCTGAAGCTGGAGAACATCATTA
ATCAAGAAATCAAAGTACTGTGCGATTTCCTGGCCACACGGAATGGAGA
GTCCATTGATTTGGCCCAGCCTCTCTCTCTGGCGATGACCAACATAGTC
AGCTTTATCTGCTTCAACTTCTCCTTCAAGAAGGGGGATCCCGCGCTGC
AGGCCATAGTGAATTTCAATGATGGCATCCTGGATGCTGTGGGCAAGGA
AATTTTGTATGACATGTTCCCTGGAATTAGGATTTTACCCAGCCAAACT
CTGGAAAACATGAAGCAGTGTGTTAGAATGCGAAACGAATTGCTGCGGG
AAATCCTTGAAAACCGTAAGGAGAACTACAGCAGAAACTCCATCACTAA
CTTGTTGGACATAATGATCCAAGCCAAGACGAACGCAGAAAGTAACACT
GGTGGCCCAGACCACAATTTAAAGCTGCTTTCAGACAGACACATGCTCG
CCACTGTTGCGGACATCTTTGGGGCCGGTGTGGAGACTTCTGCCTCTGT
GGTAAAGTGGATCGTGGCCTTCCTGCTACACTATCCTCTGCTGAGGAAG
AAGATCCAGGATGCTATCGACCAGAATATTGGTTTCAATCGTGCCCCAT
CTATCAGCGACCGGAACCAACTTGTCCTCCTGGAGGCCACCATCCGAGA
GGTGCTTCGATTCCGGCCTGTGTCCCCTACGCTCATCCCCCACAGGGCT
ATCATTGACTCCAGCATTGGCGAATTTACCATTGACAAGGACACAGATG
TCGTCGTCAATCTGTGGGCACTGCATCACAATGAGAAGGAGTGGCTCCG
GCCCGACCTGTTCATGCCTGAGCGCTTCCTGGACCCCACGGGAACCCAG
CTCATCTCACCATCATTGAGCTACTTGCCCTTCGGAGCAGGACCCCGCT
CTTGCGTAGGGGAGATGCTAGCCCGCCAGGAGCTCTTCCTCTTCACGGC
TGGATTGCTGCAGAGGTTCGACCTGGAGCTCCCAGATGATGGGCAGCTA
CCCTGTCTCGTGGGCAACCCCAGTTTGGTCCTGCAGATAGATCCTTTCA
AAGTGAAGATCAAGGAGCGCCAGGCCTGGAAGGAAGCCCACACTGAGGG
GAGTACCTCCTGA
CYP17A1-I104L
(SEQ ID NO: 34)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGAC[C]TCCTGTCAGACAACCAAAAGGGGATTGCCTT
CGCCGACCATGGTACCTCCTGGCAGCTGCATCGGAAGCTGGCACTGAGC
ACCTTTTCCCTGTTCAAGGGTGGCAACCTGAAGCTGGAGAACATCATTA
ATCAAGAAATCAAAGTACTGTGCGATTTCCTGGCCACACGGAATGGAGA
GTCCATTGATTTGGCCCAGCCTCTCTCTCTGGCGATGACCAACATAGTC
AGCTTTATCTGCTTCAACTTCTCCTTCAAGAAGGGGGATCCCGCGCTGC
AGGCCATAGTGAATTTCAATGATGGCATCCTGGATGCTGTGGGCAAGGA
AATTTTGTATGACATGTTCCCTGGAATTAGGATTTTACCCAGCCAAACT
CTGGAAAACATGAAGCAGTGTGTTAGAATGCGAAACGAATTGCTGCGGG
AAATCCTTGAAAACCGTAAGGAGAACTACAGCAGAAACTCCATCACTAA
CTTGTTGGACATAATGATCCAAGCCAAGACGAACGCAGAAAGTAACACT
GGTGGCCCAGACCACAATTTAAAGCTGCTTTCAGACAGACACATGCTCG
CCACTGTTGCGGACATCTTTGGGGCCGGTGTGGAGACTTCTGCCTCTGT
GGTAAAGTGGATCGTGGCCTTCCTGCTACACTATCCTCTGCTGAGGAAG
AAGATCCAGGATGCTATCGACCAGAATATTGGTTTCAATCGTGCCCCAT
CTATCAGCGACCGGAACCAACTTGTCCTCCTGGAGGCCACCATCCGAGA
GGTGCTTCGATTCCGGCCTGTGTCCCCTACGCTCATCCCCCACAGGGCT
ATCATTGACTCCAGCATTGGCGAATTTACCATTGACAAGGACACAGATG
TCGTCGTCAATCTGTGGGCACTGCATCACAATGAGAAGGAGTGGCTCCG
GCCCGACCTGTTCATGCCTGAGCGCTTCCTGGACCCCACGGGAACCCAG
CTCATCTCACCATCATTGAGCTACTTGCCCTTCGGAGCAGGACCCCGCT
CTTGCGTAGGGGAGATGCTAGCCCGCCAGGAGCTCTTCCTCTTCACGGC
TGGATTGCTGCAGAGGTTCGACCTGGAGCTCCCAGATGATGGGCAGCTA
CCCTGTCTCGTGGGCAACCCCAGTTTGGTCCTGCAGATAGATCCTTTCA
AAGTGAAGATCAAGGAGCGCCAGGCCTGGAAGGAAGCCCACACTGAGGG
GAGTACCTCCTGA
CYP17A1-S106A
(SEQ ID NO: 35)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTG[G]CAGACAACCAAAAGGGGATTGCCTT
CGCCGACCATGGTACCTCCTGGCAGCTGCATCGGAAGCTGGCACTGAGC
ACCTTTTCCCTGTTCAAGGGTGGCAACCTGAAGCTGGAGAACATCATTA
ATCAAGAAATCAAAGTACTGTGCGATTTCCTGGCCACACGGAATGGAGA
GTCCATTGATTTGGCCCAGCCTCTCTCTCTGGCGATGACCAACATAGTC
AGCTTTATCTGCTTCAACTTCTCCTTCAAGAAGGGGGATCCCGCGCTGC
AGGCCATAGTGAATTTCAATGATGGCATCCTGGATGCTGTGGGCAAGGA
AATTTTGTATGACATGTTCCCTGGAATTAGGATTTTACCCAGCCAAACT
CTGGAAAACATGAAGCAGTGTGTTAGAATGCGAAACGAATTGCTGCGGG
AAATCCTTGAAAACCGTAAGGAGAACTACAGCAGAAACTCCATCACTAA
CTTGTTGGACATAATGATCCAAGCCAAGACGAACGCAGAAAGTAACACT
GGTGGCCCAGACCACAATTTAAAGCTGCTTTCAGACAGACACATGCTCG
CCACTGTTGCGGACATCTTTGGGGCCGGTGTGGAGACTTCTGCCTCTGT
GGTAAAGTGGATCGTGGCCTTCCTGCTACACTATCCTCTGCTGAGGAAG
AAGATCCAGGATGCTATCGACCAGAATATTGGTTTCAATCGTGCCCCAT
CTATCAGCGACCGGAACCAACTTGTCCTCCTGGAGGCCACCATCCGAGA
GGTGCTTCGATTCCGGCCTGTGTCCCCTACGCTCATCCCCCACAGGGCT
ATCATTGACTCCAGCATTGGCGAATTTACCATTGACAAGGACACAGATG
TCGTCGTCAATCTGTGGGCACTGCATCACAATGAGAAGGAGTGGCTCCG
GCCCGACCTGTTCATGCCTGAGCGCTTCCTGGACCCCACGGGAACCCAG
CTCATCTCACCATCATTGAGCTACTTGCCCTTCGGAGCAGGACCCCGCT
CTTGCGTAGGGGAGATGCTAGCCCGCCAGGAGCTCTTCCTCTTCACGGC
TGGATTGCTGCAGAGGTTCGACCTGGAGCTCCCAGATGATGGGCAGCTA
CCCTGTCTCGTGGGCAACCCCAGTTTGGTCCTGCAGATAGATCCTTTCA
AAGTGAAGATCAAGGAGCGCCAGGCCTGGAAGGAAGCCCACACTGAGGG
GAGTACCTCCTGA
CYP17A1-S106D
(SEQ ID NO: 36)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTG[GAC]GACAACCAAAAGGGGATTGCCTT
CGCCGACCATGGTACCTCCTGGCAGCTGCATCGGAAGCTGGCACTGAGC
ACCTTTTCCCTGTTCAAGGGTGGCAACCTGAAGCTGGAGAACATCATTA
ATCAAGAAATCAAAGTACTGTGCGATTTCCTGGCCACACGGAATGGAGA
GTCCATTGATTTGGCCCAGCCTCTCTCTCTGGCGATGACCAACATAGTC
AGCTTTATCTGCTTCAACTTCTCCTTCAAGAAGGGGGATCCCGCGCTGC
AGGCCATAGTGAATTTCAATGATGGCATCCTGGATGCTGTGGGCAAGGA
AATTTTGTATGACATGTTCCCTGGAATTAGGATTTTACCCAGCCAAACT
CTGGAAAACATGAAGCAGTGTGTTAGAATGCGAAACGAATTGCTGCGGG
AAATCCTTGAAAACCGTAAGGAGAACTACAGCAGAAACTCCATCACTAA
CTTGTTGGACATAATGATCCAAGCCAAGACGAACGCAGAAAGTAACACT
GGTGGCCCAGACCACAATTTAAAGCTGCTTTCAGACAGACACATGCTCG
CCACTGTTGCGGACATCTTTGGGGCCGGTGTGGAGACTTCTGCCTCTGT
GGTAAAGTGGATCGTGGCCTTCCTGCTACACTATCCTCTGCTGAGGAAG
AAGATCCAGGATGCTATCGACCAGAATATTGGTTTCAATCGTGCCCCAT
CTATCAGCGACCGGAACCAACTTGTCCTCCTGGAGGCCACCATCCGAGA
GGTGCTTCGATTCCGGCCTGTGTCCCCTACGCTCATCCCCCACAGGGCT
ATCATTGACTCCAGCATTGGCGAATTTACCATTGACAAGGACACAGATG
TCGTCGTCAATCTGTGGGCACTGCATCACAATGAGAAGGAGTGGCTCCG
GCCCGACCTGTTCATGCCTGAGCGCTTCCTGGACCCCACGGGAACCCAG
CTCATCTCACCATCATTGAGCTACTTGCCCTTCGGAGCAGGACCCCGCT
CTTGCGTAGGGGAGATGCTAGCCCGCCAGGAGCTCTTCCTCTTCACGGC
TGGATTGCTGCAGAGGTTCGACCTGGAGCTCCCAGATGATGGGCAGCTA
CCCTGTCTCGTGGGCAACCCCAGTTTGGTCCTGCAGATAGATCCTTTCA
AAGTGAAGATCAAGGAGCGCCAGGCCTGGAAGGAAGCCCACACTGAGGG
GAGTACCTCCTGA
CYP17A1-NQ108QG
(SEQ ID NO: 37)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTGTCAGAC[C]A[GGG]AAAGGGGATTGCC
TTCGCCGACCATGGTACCTCCTGGCAGCTGCATCGGAAGCTGGCACTGA
GCACCTTTTCCCTGTTCAAGGGTGGCAACCTGAAGCTGGAGAACATCAT
TAATCAAGAAATCAAAGTACTGTGCGATTTCCTGGCCACACGGAATGGA
GAGTCCATTGATTTGGCCCAGCCTCTCTCTCTGGCGATGACCAACATAG
TCAGCTTTATCTGCTTCAACTTCTCCTTCAAGAAGGGGGATCCCGCGCT
GCAGGCCATAGTGAATTTCAATGATGGCATCCTGGATGCTGTGGGCAAG
GAAATTTTGTATGACATGTTCCCTGGAATTAGGATTTTACCCAGCCAAA
CTCTGGAAAACATGAAGCAGTGTGTTAGAATGCGAAACGAATTGCTGCG
GGAAATCCTTGAAAACCGTAAGGAGAACTACAGCAGAAACTCCATCACT
AACTTGTTGGACATAATGATCCAAGCCAAGACGAACGCAGAAAGTAACA
CTGGTGGCCCAGACCACAATTTAAAGCTGCTTTCAGACAGACACATGCT
CGCCACTGTTGCGGACATCTTTGGGGCCGGTGTGGAGACTTCTGCCTCT
GTGGTAAAGTGGATCGTGGCCTTCCTGCTACACTATCCTCTGCTGAGGA
AGAAGATCCAGGATGCTATCGACCAGAATATTGGTTTCAATCGTGCCCC
ATCTATCAGCGACCGGAACCAACTTGTCCTCCTGGAGGCCACCATCCGA
GAGGTGCTTCGATTCCGGCCTGTGTCCCCTACGCTCATCCCCCACAGGG
CTATCATTGACTCCAGCATTGGCGAATTTACCATTGACAAGGACACAGA
TGTCGTCGTCAATCTGTGGGCACTGCATCACAATGAGAAGGAGTGGCTC
CGGCCCGACCTGTTCATGCCTGAGCGCTTCCTGGACCCCACGGGAACCC
AGCTCATCTCACCATCATTGAGCTACTTGCCCTTCGGAGCAGGACCCCG
CTCTTGCGTAGGGGAGATGCTAGCCCGCCAGGAGCTCTTCCTCTTCACG
GCTGGATTGCTGCAGAGGTTCGACCTGGAGCTCCCAGATGATGGGCAGC
TACCCTGTCTCGTGGGCAACCCCAGTTTGGTCCTGCAGATAGATCCTTT
CAAAGTGAAGATCAAGGAGCGCCAGGCCTGGAAGGAAGCCCACACTGAG
GGGAGTACCTCCTGA
CYP17A1-I112V
(SEQ ID NO: 38)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTGTCAGACAACCAAAAGGGG[G]TTGCCTT
CGCCGACCATGGTACCTCCTGGCAGCTGCATCGGAAGCTGGCACTGAGC
ACCTTTTCCCTGTTCAAGGGTGGCAACCTGAAGCTGGAGAACATCATTA
ATCAAGAAATCAAAGTACTGTGCGATTTCCTGGCCACACGGAATGGAGA
GTCCATTGATTTGGCCCAGCCTCTCTCTCTGGCGATGACCAACATAGTC
AGCTTTATCTGCTTCAACTTCTCCTTCAAGAAGGGGGATCCCGCGCTGC
AGGCCATAGTGAATTTCAATGATGGCATCCTGGATGCTGTGGGCAAGGA
AATTTTGTATGACATGTTCCCTGGAATTAGGATTTTACCCAGCCAAACT
CTGGAAAACATGAAGCAGTGTGTTAGAATGCGAAACGAATTGCTGCGGG
AAATCCTTGAAAACCGTAAGGAGAACTACAGCAGAAACTCCATCACTAA
CTTGTTGGACATAATGATCCAAGCCAAGACGAACGCAGAAAGTAACACT
GGTGGCCCAGACCACAATTTAAAGCTGCTTTCAGACAGACACATGCTCG
CCACTGTTGCGGACATCTTTGGGGCCGGTGTGGAGACTTCTGCCTCTGT
GGTAAAGTGGATCGTGGCCTTCCTGCTACACTATCCTCTGCTGAGGAAG
AAGATCCAGGATGCTATCGACCAGAATATTGGTTTCAATCGTGCCCCAT
CTATCAGCGACCGGAACCAACTTGTCCTCCTGGAGGCCACCATCCGAGA
GGTGCTTCGATTCCGGCCTGTGTCCCCTACGCTCATCCCCCACAGGGCT
ATCATTGACTCCAGCATTGGCGAATTTACCATTGACAAGGACACAGATG
TCGTCGTCAATCTGTGGGCACTGCATCACAATGAGAAGGAGTGGCTCCG
GCCCGACCTGTTCATGCCTGAGCGCTTCCTGGACCCCACGGGAACCCAG
CTCATCTCACCATCATTGAGCTACTTGCCCTTCGGAGCAGGACCCCGCT
CTTGCGTAGGGGAGATGCTAGCCCGCCAGGAGCTCTTCCTCTTCACGGC
TGGATTGCTGCAGAGGTTCGACCTGGAGCTCCCAGATGATGGGCAGCTA
CCCTGTCTCGTGGGCAACCCCAGTTTGGTCCTGCAGATAGATCCTTTCA
AAGTGAAGATCAAGGAGCGCCAGGCCTGGAAGGAAGCCCACACTGAGGG
GAGTACCTCCTGA
CYP17A1-N202T
(SEQ ID NO: 39)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
GTTCTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTGTCAGACAACCAAAAGGGGATTGCCTTCG
CCGACCATGGTACCTCCTGGCAGCTGCATCGGAAGCTGGCACTGAGCAC
CTTTTCCCTGTTCAAGGGTGGCAACCTGAAGCTGGAGAACATCATTAAT
CAAGAAATCAAAGTACTGTGCGATTTCCTGGCCACACGGAATGGAGAGT
CCATTGATTTGGCCCAGCCTCTCTCTCTGGCGATGACCAACATAGTCAG
CTTTATCTGCTTCAACTTCTCCTTCAAGAAGGGGGATCCCGCGCTGCAG
GCCATAGTGAATTTCA[C]TGATGGCATCCTGGATGCTGTGGGCAAGGA
AATTTTGTATGACATGTTCCCTGGAATTAGGATTTTACCCAGCCAAACT
CTGGAAAACATGAAGCAGTGTGTTAGAATGCGAAACGAATTGCTGCGGG
AAATCCTTGAAAACCGTAAGGAGAACTACAGCAGAAACTCCATCACTAA
CTTGTTGGACATAATGATCCAAGCCAAGACGAACGCAGAAAGTAACACT
GGTGGCCCAGACCACAATTTAAAGCTGCTTTCAGACAGACACATGCTCG
CCACTGTTGCGGACATCTTTGGGGCCGGTGTGGAGACTTCTGCCTCTGT
GGTAAAGTGGATCGTGGCCTTCCTGCTACACTATCCTCTGCTGAGGAAG
AAGATCCAGGATGCTATCGACCAGAATATTGGTTTCAATCGTGCCCCAT
CTATCAGCGACCGGAACCAACTTGTCCTCCTGGAGGCCACCATCCGAGA
GGTGCTTCGATTCCGGCCTGTGTCCCCTACGCTCATCCCCCACAGGGCT
ATCATTGACTCCAGCATTGGCGAATTTACCATTGACAAGGACACAGATG
TCGTCGTCAATCTGTGGGCACTGCATCACAATGAGAAGGAGTGGCTCCG
GCCCGACCTGTTCATGCCTGAGCGCTTCCTGGACCCCACGGGAACCCAG
CTCATCTCACCATCATTGAGCTACTTGCCCTTCGGAGCAGGACCCCGCT
CTTGCGTAGGGGAGATGCTAGCCCGCCAGGAGCTCTTCCTCTTCACGGC
TGGATTGCTGCAGAGGTTCGACCTGGAGCTCCCAGATGATGGGCAGCTA
CCCTGTCTCGTGGGCAACCCCAGTTTGGTCCTGCAGATAGATCCTTTCA
AAGTGAAGATCAAGGAGCGCCAGGCCTGGAAGGAAGCCCACACTGAGGG
GAGTACCTCCTGA
CYP17A1-IS344FN
(SEQ ID NO: 40)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTGTCAGACAACCAAAAGGGGATTGCCTTCG
CCGACCATGGTACCTCCTGGCAGCTGCATCGGAAGCTGGCACTGAGCAC
CTTTTCCCTGTTCAAGGGTGGCAACCTGAAGCTGGAGAACATCATTAAT
CAAGAAATCAAAGTACTGTGCGATTTCCTGGCCACACGGAATGGAGAGT
CCATTGATTTGGCCCAGCCTCTCTCTCTGGCGATGACCAACATAGTCAG
CTTTATCTGCTTCAACTTCTCCTTCAAGAAGGGGGATCCCGCGCTGCAG
GCCATAGTGAATTTCAATGATGGCATCCTGGATGCTGTGGGCAAGGAAA
TTTTGTATGACATGTTCCCTGGAATTAGGATTTTACCCAGCCAAACTCT
GGAAAACATGAAGCAGTGTGTTAGAATGCGAAACGAATTGCTGCGGGAA
ATCCTTGAAAACCGTAAGGAGAACTACAGCAGAAACTCCATCACTAACT
TGTTGGACATAATGATCCAAGCCAAGACGAACGCAGAAAGTAACACTGG
TGGCCCAGACCACAATTTAAAGCTGCTTTCAGACAGACACATGCTCGCC
ACTGTTGCGGACATCTTTGGGGCCGGTGTGGAGACTTCTGCCTCTGTGG
TAAAGTGGATCGTGGCCTTCCTGCTACACTATCCTCTGCTGAGGAAGAA
GATCCAGGATGCTATCGACCAGAATATTGGTTTCAATCGTGCCCCATCT
[T]TCA[A]CGACCGGAACCAACTTGTCCTCCTGGAGGCCACCATCCGA
GAGGTGCTTCGATTCCGGCCTGTGTCCCCTACGCTCATCCCCCACAGGG
CTATCATTGACTCCAGCATTGGCGAATTTACCATTGACAAGGACACAGA
TGTCGTCGTCAATCTGTGGGCACTGCATCACAATGAGAAGGAGTGGCTC
CGGCCCGACCTGTTCATGCCTGAGCGCTTCCTGGACCCCACGGGAACCC
AGCTCATCTCACCATCATTGAGCTACTTGCCCTTCGGAGCAGGACCCCG
CTCTTGCGTAGGGGAGATGCTAGCCCGCCAGGAGCTCTTCCTCTTCACG
GCTGGATTGCTGCAGAGGTTCGACCTGGAGCTCCCAGATGATGGGCAGC
TACCCTGTCTCGTGGGCAACCCCAGTTTGGTCCTGCAGATAGATCCTTT
CAAAGTGAAGATCAAGGAGCGCCAGGCCTGGAAGGAAGCCCACACTGAG
GGGAGTACCTCCTGA
CYP17A1-N348S
(SEQ ID NO: 41)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTGTCAGACAACCAAAAGGGGATTGCCTTCG
CCGACCATGGTACCTCCTGGCAGCTGCATCGGAAGCTGGCACTGAGCAC
CTTTTCCCTGTTCAAGGGTGGCAACCTGAAGCTGGAGAACATCATTAAT
AAAGTACTGTGCGATTTCCTGCAAGAAATCGCCACACGGAATGGAGAGT
CCATTGATTTGGCCCAGCCTCTCTCTCTGGCGATGACCAACATAGTCAG
CTTTATCTGCTTCAACTTCTCCTTCAAGAAGGGGGATCCCGCGCTGCAG
GCCATAGTGAATTTCAATGATGGCATCCTGGATGCTGTGGGCAAGGAAA
TTTTGTATGACATGTTCCCTGGAATTAGGATTTTACCCAGCCAAACTCT
GGAAAACATGAAGCAGTGTGTTAGAATGCGAAACGAATTGCTGCGGGAA
ATCCTTGAAAACCGTAAGGAGAACTACAGCAGAAACTCCATCACTAACT
TGTTGGACATAATGATCCAAGCCAAGACGAACGCAGAAAGTAACACTGG
TGGCCCAGACCACAATTTAAAGCTGCTTTCAGACAGACACATGCTCGCC
ACTGTTGCGGACATCTTTGGGGCCGGTGTGGAGACTTCTGCCTCTGTGG
TAAAGTGGATCGTGGCCTTCCTGCTACACTATCCTCTGCTGAGGAAGAA
GATCCAGGATGCTATCGACCAGAATATTGGTTTCAATCGTGCCCCATCT
ATCAGCGACCGGA[G]CCAACTTGTCCTCCTGGAGGCCACCATCCGAGA
ATCATTGACTCCAGCATTGGCGAATGGTGCTTCGATTCCGGCCTGTGTC
CCCTACGCTCATCCCCCACAGGGCTTTACCATTGACAAGGACACAGATG
TCGTCGTCAATCTGTGGGCACTGCATCACAATGAGAAGGAGTGGCTCCG
GCCCGACCTGTTCATGCCTGAGCGCTTCCTGGACCCCACGGGAACCCAG
CTCATCTCACCATCATTGAGCTACTTGCCCTTCGGAGCAGGACCCCGCT
CTTGCGTAGGGGAGATGCTAGCCCGCCAGGAGCTCTTCCTCTTCACGGC
TGGATTGCTGCAGAGGTTCGACCTGGAGCTCCCAGATGATGGGCAGCTA
CCCTGTCTCGTGGGCAACCCCAGTTTGGTCCTGCAGATAGATCCTTTCA
AAGTGAAGATCAAGGAGCGCCAGGCCTGGAAGGAAGCCCACACTGAGGG
GAGTACCTCCTGA
CYP17A1-L352M
(SEQ ID NO: 42)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTGTCAGACAACCAAAAGGGGATTGCCTTCG
CCGACCATGGTACCTCCTGGCAGCTGCATCGGAAGCTGGCACTGAGCAC
CTTTTCCCTGTTCAAGGGTGGCAACCTGAAGCTGGAGAACATCATTAAT
CAAGAAATCAAAGTACTGTGCGATTTCCTGGCCACACGGAATGGAGAGT
CCATTGATTTGGCCCAGCCTCTCTCTCTGGCGATGACCAACATAGTCAG
CTTTATCTGCTTCAACTTCTCCTTCAAGAAGGGGGATCCCGCGCTGCAG
GCCATAGTGAATTTCAATGATGGCATCCTGGATGCTGTGGGCAAGGAAA
TTTTGTATGACATGTTCCCTGGAATTAGGATTTTACCCAGCCAAACTCT
GGAAAACATGAAGCAGTGTGTTAGAATGCGAAACGAATTGCTGCGGGAA
ATCCTTGAAAACCGTAAGGAGAACTACAGCAGAAACTCCATCACTAACT
TGTTGGACATAATGATCCAAGCCAAGACGAACGCAGAAAGTAACACTGG
TGGCCCAGACCACAATTTAAAGCTGCTTTCAGACAGACACATGCTCGCC
ACTGTTGCGGACATCTTTGGGGCCGGTGTGGAGACTTCTGCCTCTGTGG
TAAAGTGGATCGTGGCCTTCCTGCTACACTATCCTCTGCTGAGGAAGAA
GATCCAGGATGCTATCGACCAGAATATTGGTTTCAATCGTGCCCCATCT
ATCAGCGACCGGAACCAACTTGTC[A]T[G]CTGGAGGCCACCATCCGA
GAGGTGCTTCGATTCCGGCCTGTGTCCCCTACGCTCATCCCCCACAGGG
CTATCATTGACTCCAGCATTGGCGAATTTACCATTGACAAGGACACAGA
TGTCGTCGTCAATCTGTGGGCACTGCATCACAATGAGAAGGAGTGGCTC
CGGCCCGACCTGTTCATGCCTGAGCGCTTCCTGGACCCCACGGGAACCC
AGCTCATCTCACCATCATTGAGCTACTTGCCCTTCGGAGCAGGACCCCG
CTCTTGCGTAGGGGAGATGCTAGCCCGCCAGGAGCTCTTCCTCTTCACG
GCTGGATTGCTGCAGAGGTTCGACCTGGAGCTCCCAGATGATGGGCAGC
TACCCTGTCTCGTGGGCAACCCCAGTTTGGTCCTGCAGATAGATCCTTT
CAAAGTGAAGATCAAGGAGCGCCAGGCCTGGAAGGAAGCCCACACTGAG
GGGAGTACCTCCTGA
CYP17A1-L454V
(SEQ ID NO: 43)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTAGACATCCTGTCAGACAACCAAAAGGGGATTGCCTTCG
CCGACCATGGTACCTCCTGGCAGCTGCATCGGAAGCTGGCACTGAGCAC
CTTTTCCCTGTTCAAGGGTGGCAACCTGAAGCTGGAGAACATCATTAAT
CAAGAAATCAAAGTACTGTGCGATTTCCTGGCCACACGGAATGGAGAGT
CCATTGATTTGGCCCAGCCTCTCTCTCTGGCGATGACCAACATAGTCAG
TCAACTTCTCTTTATCTGCTCCTTCAAGAAGGGGGATCCCGCGCTGCAG
GCCATAGTGAATTTCAATGATGGCATCCTGGATGCTGTGGGCAAGGAAA
TTTTGTATGACATGTTCCCTGGAATTAGGATTTTACCCAGCCAAACTCT
GGAAAACATGAAGCAGTGTGTTAGAATGCGAAACGAATTGCTGCGGGAA
ATCCTTGAAAACCGTAAGGAGAACTACAGCAGAAACTCCATCACTAACT
TGTTGGACATAATGATCCAAGCCAAGACGAACGCAGAAAGTAACACTGG
TGGCCCAGACCACAATTTAAAGCTGCTTTCAGACAGACACATGCTCGCC
ACTGTTGCGGACATCTTTGGGGCCGGTGTGGAGACTTCTGCCTCTGTGG
TAAAGTGGATCGTGGCCTTCCTGCTACACTATCCTCTGCTGAGGAAGAA
GATCCAGGATGCTATCGACCAGAATATTGGTTTCAATCGTGCCCCATCT
ATCAGCGACCGGAACCAACTTGTCCTCCTGGAGGCCACCATCCGAGAGG
TGCTTCGATTCCGGCCTGTGTCCCCTACGCTCATCCCCCACAGGGCTAT
CATTGACTCCAGCATTGGCGAATTTACCATTGACAAGGACACAGATGTC
GTCGTCAATCTGTGGGCACTGCATCACAATGAGAAGGAGTGGCTCCGGC
CCGACCTGTTCATGCCTGAGCGCTTCCTGGACCCCACGGGAACCCAGCT
CATCTCACCATCATTGAGCTACTTGCCCTTCGGAGCAGGACCCCGCTCT
TGCGTAGGGGAGATGCTAGCCCGCCAGGAGCTCTTC[G]TCTTCACGGC
TGGATTGCTGCAGAGGTTCGACCTGGAGCTCCCAGATGATGGGCAGCTA
CCCTGTCTCGTGGGCAACCCCAGTTTGGTCCTGCAGATAGATCCTTTCA
AAGTGAAGATCAAGGAGCGCCAGGCCTGGAAGGAAGCCCACACTGAGGG
GAGTACCTCCTGA
CYP17A1-L102Q + 112V (DM)
(SEQ ID NO: 44)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTC[A]AGACATCCTGTCAGACAACCAAAAGGGG[G]TTGCC
TTCGCCGACCATGGTACCTCCTGGCAGCTGCATCGGAAGCTGGCACTGA
GCACCTTTTCCCTGTTCAAGGGTGGCAACCTGAAGCTGGAGAACATCAT
TAATCAAGAAATCAAAGTACTGTGCGATTTCCTGGCCACACGGAATGGA
GAGTCCATTGATTTGGCCCAGCCTCTCTCTCTGGCGATGACCAACATAG
TCAGCTTTATCTGCTTCAACTTCTCCTTCAAGAAGGGGGATCCCGCGCT
GCAGGCCATAGTGAATTTCAATGATGGCATCCTGGATGCTGTGGGCAAG
GAAATTTTGTATGACATGTTCCCTGGAATTAGGATTTTACCCAGCCAAA
CTCTGGAAAACATGAAGCAGTGTGTTAGAATGCGAAACGAATTGCTGCG
GGAAATCCTTGAAAACCGTAAGGAGAACTACAGCAGAAACTCCATCACT
AACTTGTTGGACATAATGATCCAAGCCAAGACGAACGCAGAAAGTAACA
CTGGTGGCCCAGACCACAATTTAAAGCTGCTTTCAGACAGACACATGCT
CGCCACTGTTGCGGACATCTTTGGGGCCGGTGTGGAGACTTCTGCCTCT
GTGGTAAAGTGGATCGTGGCCTTCCTGCTACACTATCCTCTGCTGAGGA
AGAAGATCCAGGATGCTATCGACCAGAATATTGGTTTCAATCGTGCCCC
ATCTATCAGCGACCGGAACCAACTTGTCCTCCTGGAGGCCACCATCCGA
GAGGTGCTTCGATTCCGGCCTGTGTCCCCTACGCTCATCCCCCACAGGG
CTATCATTGACTCCAGCATTGGCGAATTTACCATTGACAAGGACACAGA
TGTCGTCGTCAATCTGTGGGCACTGCATCACAATGAGAAGGAGTGGCTC
CGGCCCGACCTGTTCATGCCTGAGCGCTTCCTGGACCCCACGGGAACCC
AGCTCATCTCACCATCATTGAGCTACTTGCCCTTCGGAGCAGGACCCCG
CTCTTGCGTAGGGGAGATGCTAGCCCGCCAGGAGCTCTTCCTCTTCACG
GCTGGATTGCTGCAGAGGTTCGACCTGGAGCTCCCAGATGATGGGCAGC
TACCCTGTCTCGTGGGCAACCCCAGTTTGGTCCTGCAGATAGATCCTTT
CAAAGTGAAGATCAAGGAGCGCCAGGCCTGGAAGGAAGCCCACACTGAG
GGGAGTACCTCCTGA
CYP17A1-L102Q + D103S + I112V (TM)
(SEQ ID NO: 45)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTC[A]A[TC]CATCCTGTCAGACAACCAAAAGGGG[G]TTG
CCTTCGCCGACCATGGTACCTCCTGGCAGCTGCATCGGAAGCTGGCACT
GAGCACCTTTTCCCTGTTCAAGGGTGGCAACCTGAAGCTGGAGAACATC
ATTAATCAAGAAATCAAAGTACTGTGCGATTTCCTGGCCACACGGAATG
GAGAGTCCATTGATTTGGCCCAGCCTCTCTCTCTGGCGATGACCAACAT
AGTCAGCTTTATCTGCTTCAACTTCTCCTTCAAGAAGGGGGATCCCGCG
CTGCAGGCCATAGTGAATTTCAATGATGGCATCCTGGATGCTGTGGGCA
AGGAAATTTTGTATGACATGTTCCCTGGAATTAGGATTTTACCCAGCCA
AACTCTGGAAAACATGAAGCAGTGTGTTAGAATGCGAAACGAATTGCTG
CGGGAAATCCTTGAAAACCGTAAGGAGAACTACAGCAGAAACTCCATCA
CTAACTTGTTGGACATAATGATCCAAGCCAAGACGAACGCAGAAAGTAA
CACTGGTGGCCCAGACCACAATTTAAAGCTGCTTTCAGACAGACACATG
CTCGCCACTGTTGCGGACATCTTTGGGGCCGGTGTGGAGACTTCTGCCT
CTGTGGTAAAGTGGATCGTGGCCTTCCTGCTACACTATCCTCTGCTGAG
GAAGAAGATCCAGGATGCTATCGACCAGAATATTGGTTTCAATCGTGCC
CCATCTATCAGCGACCGGAACCAACTTGTCCTCCTGGAGGCCACCATCC
GAGAGGTGCTTCGATTCCGGCCTGTGTCCCCTACGCTCATCCCCCACAG
GGCTATCATTGACTCCAGCATTGGCGAATTTACCATTGACAAGGACACA
GATGTCGTCGTCAATCTGTGGGCACTGCATCACAATGAGAAGGAGTGGC
TCCGGCCCGACCTGTTCATGCCTGAGCGCTTCCTGGACCCCACGGGAAC
CCAGCTCATCTCACCATCATTGAGCTACTTGCCCTTCGGAGCAGGACCC
CGCTCTTGCGTAGGGGAGATGCTAGCCCGCCAGGAGCTCTTCCTCTTCA
CGGCTGGATTGCTGCAGAGGTTCGACCTGGAGCTCCCAGATGATGGGCA
GCTACCCTGTCTCGTGGGCAACCCCAGTTTGGTCCTGCAGATAGATCCT
TTCAAAGTGAAGATCAAGGAGCGCCAGGCCTGGAAGGAAGCCCACACTG
AGGGGAGTACCTCCTGA
CYP17A1-SM L102Q + D103S + I104L + NQ108QG + I112V
(SEQ ID NO: 46)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTC[A]A[TC]C[C]TCCTGTCAGAC[C]A[GGG]AAAGGGG
[G]TTGCCTTCGCCGACCATGGTACCTCCTGGCAGCTGCATCGGAAGCT
GGCACTGAGCACCTTTTCCCTGTTCAAGGGTGGCAACCTGAAGCTGGAG
AACATCATTAATCAAGAAATCAAAGTACTGTGCGATTTCCTGGCCACAC
GGAATGGAGAGTCCATTGATTTGGCCCAGCCTCTCTCTCTGGCGATGAC
CAACATAGTCAGCTTTATCTGCTTCAACTTCTCCTTCAAGAAGGGGGAT
CCCGCGCTGCAGGCCATAGTGAATTTCAATGATGGCATCCTGGATGCTG
TGGGCAAGGAAATTTTGTATGACATGTTCCCTGGAATTAGGATTTTACC
CAGCCAAACTCTGGAAAACATGAAGCAGTGTGTTAGAATGCGAAACGAA
TTGCTGCGGGAAATCCTTGAAAACCGTAAGGAGAACTACAGCAGAAACT
CCATCACTAACTTGTTGGACATAATGATCCAAGCCAAGACGAACGCAGA
AAGTAACACTGGTGGCCCAGACCACAATTTAAAGCTGCTTTCAGACAGA
CACATGCTCGCCACTGTTGCGGACATCTTTGGGGCCGGTGTGGAGACTT
CTGCCTCTGTGGTAAAGTGGATCGTGGCCTTCCTGCTACACTATCCTCT
GCTGAGGAAGAAGATCCAGGATGCTATCGACCAGAATATTGGTTTCAAT
CGTGCCCCATCTATCAGCGACCGGAACCAACTTGTCCTCCTGGAGGCCA
CCATCCGAGAGGTGCTTCGATTCCGGCCTGTGTCCCCTACGCTCATCCC
CCACAGGGCTATCATTGACTCCAGCATTGGCGAATTTACCATTGACAAG
GACACAGATGTCGTCGTCAATCTGTGGGCACTGCATCACAATGAGAAGG
AGTGGCTCCGGCCCGACCTGTTCATGCCTGAGCGCTTCCTGGACCCCAC
GGGAACCCAGCTCATCTCACCATCATTGAGCTACTTGCCCTTCGGAGCA
GGACCCCGCTCTTGCGTAGGGGAGATGCTAGCCCGCCAGGAGCTCTTCC
TCTTCACGGCTGGATTGCTGCAGAGGTTCGACCTGGAGCTCCCAGATGA
TGGGCAGCTACCCTGTCTCGTGGGCAACCCCAGTTTGGTCCTGCAGATA
GATCCTTTCAAAGTGAAGATCAAGGAGCGCCAGGCCTGGAAGGAAGCCC
ACACTGAGGGGAGTACCTCCTGA
CYP17A1 D103S + L454V
(SEQ ID NO: 47)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTA[TC]CATCCTGTCAGACAACCAAAAGGGGATTGCCTT
CGCCGACCATGGTACCTCCTGGCAGCTGCATCGGAAGCTGGCACTGAGC
ACCTTTTCCCTGTTCAAGGGTGGCAACCTGAAGCTGGAGAACATCATTA
ATCAAGAAATCAAAGTACTGTGCGATTTCCTGGCCACACGGAATGGAGA
GTCCATTGATTTGGCCCAGCCTCTCTCTCTGGCGATGACCAACATAGTC
AGCTTTATCTGCTTCAACTTCTCCTTCAAGAAGGGGGATCCCGCGCTGC
AGGCCATAGTGAATTTCAATGATGGCATCCTGGATGCTGTGGGCAAGGA
AATTTTGTATGACATGTTCCCTGGAATTAGGATTTTACCCAGCCAAACT
CTGGAAAACATGAAGCAGTGTGTTAGAATGCGAAACGAATTGCTGCGGG
AAATCCTTGAAAACCGTAAGGAGAACTACAGCAGAAACTCCATCACTAA
CTTGTTGGACATAATGATCCAAGCCAAGACGAACGCAGAAAGTAACACT
GGTGGCCCAGACCACAATTTAAAGCTGCTTTCAGACAGACACATGCTCG
CCACTGTTGCGGACATCTTTGGGGCCGGTGTGGAGACTTCTGCCTCTGT
GGTAAAGTGGATCGTGGCCTTCCTGCTACACTATCCTCTGCTGAGGAAG
AAGATCCAGGATGCTATCGACCAGAATATTGGTTTCAATCGTGCCCCAT
CTATCAGCGACCGGAACCAACTTGTCCTCCTGGAGGCCACCATCCGAGA
GGTGCTTCGATTCCGGCCTGTGTCCCCTACGCTCATCCCCCACAGGGCT
ATCATTGACTCCAGCATTGGCGAATTTACCATTGACAAGGACACAGATG
TCGTCGTCAATCTGTGGGCACTGCATCACAATGAGAAGGAGTGGCTCCG
GCCCGACCTGTTCATGCCTGAGCGCTTCCTGGACCCCACGGGAACCCAG
CTCATCTCACCATCATTGAGCTACTTGCCCTTCGGAGCAGGACCCCGCT
CTTGCGTAGGGGAGATGCTAGCCCGCCAGGAGCTCTTC[G]TCTTCACG
GCTGGATTGCTGCAGAGGTTCGACCTGGAGCTCCCAGATGATGGGCAGC
TACCCTGTCTCGTGGGCAACCCCAGTTTGGTCCTGCAGATAGATCCTTT
CAAAGTGAAGATCAAGGAGCGCCAGGCCTGGAAGGAAGCCCACACTGAG
GGGAGTACCTCCTGA
CYP17A1 D103S + I104L + L454V (IDL)
(SEQ ID NO: 48)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTA[TC]C[C]TCCTGTCAGACAACCAAAAGGGGATTGCC
TTCGCCGACCATGGTACCTCCTGGCAGCTGCATCGGAAGCTGGCACTGA
GCACCTTTTCCCTGTTCAAGGGTGGCAACCTGAAGCTGGAGAACATCAT
TAATCAAGAAATCAAAGTACTGTGCGATTTCCTGGCCACACGGAATGGA
GAGTCCATTGATTTGGCCCAGCCTCTCTCTCTGGCGATGACCAACATAG
TCAGCTTTATCTGCTTCAACTTCTCCTTCAAGAAGGGGGATCCCGCGCT
GCAGGCCATAGTGAATTTCAATGATGGCATCCTGGATGCTGTGGGCAAG
GAAATTTTGTATGACATGTTCCCTGGAATTAGGATTTTACCCAGCCAAA
CTCTGGAAAACATGAAGCAGTGTGTTAGAATGCGAAACGAATTGCTGCG
GGAAATCCTTGAAAACCGTAAGGAGAACTACAGCAGAAACTCCATCACT
AACTTGTTGGACATAATGATCCAAGCCAAGACGAACGCAGAAAGTAACA
CTGGTGGCCCAGACCACAATTTAAAGCTGCTTTCAGACAGACACATGCT
CGCCACTGTTGCGGACATCTTTGGGGCCGGTGTGGAGACTTCTGCCTCT
GTGGTAAAGTGGATCGTGGCCTTCCTGCTACACTATCCTCTGCTGAGGA
AGAAGATCCAGGATGCTATCGACCAGAATATTGGTTTCAATCGTGCCCC
ATCTATCAGCGACCGGAACCAACTTGTCCTCCTGGAGGCCACCATCCGA
GAGGTGCTTCGATTCCGGCCTGTGTCCCCTACGCTCATCCCCCACAGGG
CTATCATTGACTCCAGCATTGGCGAATTTACCATTGACAAGGACACAGA
TGTCGTCGTCAATCTGTGGGCACTGCATCACAATGAGAAGGAGTGGCTC
CGGCCCGACCTGTTCATGCCTGAGCGCTTCCTGGACCCCACGGGAACCC
AGCTCATCTCACCATCATTGAGCTACTTGCCCTTCGGAGCAGGACCCCG
CTCTTGCGTAGGGGAGATGCTAGCCCGCCAGGAGCTCTTC[G]TCTTCA
CGGCTGGATTGCTGCAGAGGTTCGACCTGGAGCTCCCAGATGATGGGCA
GCTACCCTGTCTCGTGGGCAACCCCAGTTTGGTCCTGCAGATAGATCCT
TTCAAAGTGAAGATCAAGGAGCGCCAGGCCTGGAAGGAAGCCCACACTG
AGGGGAGTACCTCCTGA
CYP17A1 D103S + S106A + L454V (SDL)
(SEQ ID NO: 49)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTCTA[TC]CATCCTG[G]CAGACAACCAAAAGGGGATTGCC
TTCGCCGACCATGGTACCTCCTGGCAGCTGCATCGGAAGCTGGCACTGA
GCACCTTTTCCCTGTTCAAGGGTGGCAACCTGAAGCTGGAGAACATCAT
TAATCAAGAAATCAAAGTACTGTGCGATTTCCTGGCCACACGGAATGGA
GAGTCCATTGATTTGGCCCAGCCTCTCTCTCTGGCGATGACCAACATAG
TCAGCTTTATCTGCTTCAACTTCTCCTTCAAGAAGGGGGATCCCGCGCT
GCAGGCCATAGTGAATTTCAATGATGGCATCCTGGATGCTGTGGGCAAG
GAAATTTTGTATGACATGTTCCCTGGAATTAGGATTTTACCCAGCCAAA
CTCTGGAAAACATGAAGCAGTGTGTTAGAATGCGAAACGAATTGCTGCG
GGAAATCCTTGAAAACCGTAAGGAGAACTACAGCAGAAACTCCATCACT
AACTTGTTGGACATAATGATCCAAGCCAAGACGAACGCAGAAAGTAACA
CTGGTGGCCCAGACCACAATTTAAAGCTGCTTTCAGACAGACACATGCT
CGCCACTGTTGCGGACATCTTTGGGGCCGGTGTGGAGACTTCTGCCTCT
GTGGTAAAGTGGATCGTGGCCTTCCTGCTACACTATCCTCTGCTGAGGA
AGAAGATCCAGGATGCTATCGACCAGAATATTGGTTTCAATCGTGCCCC
ATCTATCAGCGACCGGAACCAACTTGTCCTCCTGGAGGCCACCATCCGA
GAGGTGCTTCGATTCCGGCCTGTGTCCCCTACGCTCATCCCCCACAGGG
CTATCATTGACTCCAGCATTGGCGAATTTACCATTGACAAGGACACAGA
TGTCGTCGTCAATCTGTGGGCACTGCATCACAATGAGAAGGAGTGGCTC
CGGCCCGACCTGTTCATGCCTGAGCGCTTCCTGGACCCCACGGGAACCC
AGCTCATCTCACCATCATTGAGCTACTTGCCCTTCGGAGCAGGACCCCG
CTCTTGCGTAGGGGAGATGCTAGCCCGCCAGGAGCTCTTC[G]TCTTCA
CGGCTGGATTGCTGCAGAGGTTCGACCTGGAGCTCCCAGATGATGGGCA
GCTACCCTGTCTCGTGGGCAACCCCAGTTTGGTCCTGCAGATAGATCCT
TTCAAAGTGAAGATCAAGGAGCGCCAGGCCTGGAAGGAAGCCCACACTG
AGGGGAGTACCTCCTGA
CYP17A1-(STM) S106A + L102Q + D102S + L112V
(SEQ ID NO: 50)
ATGTGGGTGCTCTTGGTTTTCTTCTTGCTCACCCTCACCTATTTATTTT
GGCCTAAGACCAAGGGCTCTGGTGCCAAGTACCCCAGGAGTCTCCCAGT
CCTGCCCGTGGTGGGCAGCCTGCCATTCCTACCCAGACGTGGCCACCAG
CACATGAACTTCTTCAAGTTGCAGGACAAATATGGCCCCATCTTCTCCT
TTCGTCTGGGTTCCAAGACTACCGTGGTAATTGGTGACCACCAGCTGGC
CAAGGAGGTGCTTCTCAAGAAGGGCAAGGAATTCTCCGGGCGGCCCAGA
GTGATGACTC[A]A[TC]CATCCTG[G]CAGACAACCAAAAGGGG[G]T
TGCCTTCGCCGACCATGGTACCTCCTGGCAGCTGCATCGGAAGCTGGCA
CTGAGCACCTTTTCCCTGTTCAAGGGTGGCAACCTGAAGCTGGAGAACA
TCATTAATCAAGAAATCAAAGTACTGTGCGATTTCCTGGCCACACGGAA
TGGAGAGTCCATTGATTTGGCCCAGCCTCTCTCTCTGGCGATGACCAAC
ATAGTCAGCTTTATCTGCTTCAACTTCTCCTTCAAGAAGGGGGATCCCG
CGCTGCAGGCCATAGTGAATTTCAATGATGGCATCCTGGATGCTGTGGG
CAAGGAAATTTTGTATGACATGTTCCCTGGAATTAGGATTTTACCCAGC
CAAACTCTGGAAAACATGAAGCAGTGTGTTAGAATGCGAAACGAATTGC
TGCGGGAAATCCTTGAAAACCGTAAGGAGAACTACAGCAGAAACTCCAT
CACTAACTTGTTGGACATAATGATCCAAGCCAAGACGAACGCAGAAAGT
AACACTGGTGGCCCAGACCACAATTTAAAGCTGCTTTCAGACAGACACA
TGCTCGCCACTGTTGCGGACATCTTTGGGGCCGGTGTGGAGACTTCTGC
CTCTGTGGTAAAGTGGATCGTGGCCTTCCTGCTACACTATCCTCTGCTG
AGGAAGAAGATCCAGGATGCTATCGACCAGAATATTGGTTTCAATCGTG
CCCCATCTATCAGCGACCGGAACCAACTTGTCCTCCTGGAGGCCACCAT
CCGAGAGGTGCTTCGATTCCGGCCTGTGTCCCCTACGCTCATCCCCCAC
AGGGCTATCATTGACTCCAGCATTGGCGAATTTACCATTGACAAGGACA
CAGATGTCGTCGTCAATCTGTGGGCACTGCATCACAATGAGAAGGAGTG
GCTCCGGCCCGACCTGTTCATGCCTGAGCGCTTCCTGGACCCCACGGGA
ACCCAGCTCATCTCACCATCATTGAGCTACTTGCCCTTCGGAGCAGGAC
CCCGCTCTTGCGTAGGGGAGATGCTAGCCCGCCAGGAGCTCTTCCTCTT
CACGGCTGGATTGCTGCAGAGGTTCGACCTGGAGCTCCCAGATGATGGG
CAGCTACCCTGTCTCGTGGGCAACCCCAGTTTGGTCCTGCAGATAGATC
CTTTCAAAGTGAAGATCAAGGAGCGCCAGGCCTGGAAGGAAGCCCACAC
TGAGGGGAGTACCTCCTGA

What is claimed:

1. An altered CYP17 protein that modifies 16-androstene steroid activity or production in pigs, said protein comprising:
a modification of the amino acid present at one or more positions selected from the group consisting of: amino acids 102, 103, 104, 106, 108, 109, 112, 202, 344, 345, 348, 352 and 454 of the porcine CYP17 protein, compared to wild type (SEQ ID NO:4).

2. The altered CYP17 protein of claim 1 wherein said modification comprises one of more of:
(a) a glutamine residue at position 102;
(b) a serine residue position 103;
(c) a leucine residue at position 104;
(d) an alanine residue at position 106;
(e) an aspartic acid residue at position 106;
(f) a glutamine residue at position 108;
(g) a glycine residue at position 109;
(h) a valine residue at position 112;
(i) a threonine residue at position 202;
(j) a phenylalanine residue at position 344;
(k) an asparagine residue at position 345;
(l) a serine residue at position 348;
(m) a methionine residue at position 352; and
(n) a valine residue at position 454.

3. A method of modifying 16-androstene steroid activity to reducing boar taint in a pig comprising:
introducing to said pig a polynucleotide that encodes a CYP17 protein, wherein amino acid 102, 103, 104, 106, 108, 112, 202, 344, 345, 348, 352 and 454 of the porcine CYP17 protein is modified from wild type (SEQ ID NO:4).

4. The method of claim 3 wherein said modification comprises one of more of:
(a) a glutamine residue at position 102;
(b) a serine residue position 103;
(c) a leucine residue at position 104;
(d) an alanine residue at position 106;
(e) an aspartic acid residue at position 106;
(f) a glutamine residue at position 108;
(g) a glycine residue at position 109;
(h) a valine residue at position 112;
(i) a threonine residue at position 202;
(j) a phenylalanine residue at position 344;
(k) an asparagine residue at position 345;
(l) a serine residue at position 348;
(m) a methionine residue at position 352; and
(n) a valine residue at position 454.

5. A genetically engineered pig having modified 16-androstene steroid activity said pig comprising:
a modified CYP17 protein, wherein said modification includes a change at one or more positions selected from the group consisting of: amino acid 102, 103, 104, 106, 108, 112, 202, 344, 345, 348, 352 and 454 of the porcine CYP17 protein compared to wild type (SEQ ID NO:4).

6. The genetically engineered pig of claim 5 wherein said modified CYP17 protein comprises one or more of:
(a) a glutamine residue at position 102;
(b) a serine residue position 103;
(c) a leucine residue at position 104;
(d) an alanine residue at position 106;
(e) an aspartic acid residue at position 106;
(f) a glutamine residue at position 108;
(g) a glycine residue at position 109;
(h) a valine residue at position 112;
(i) a threonine residue at position 202;
(j) a phenylalanine residue at position 344;
(k) an asparagine residue at position 345;
(l) a serine residue at position 348;
(m) a methionine residue at position 352; and
(n) a valine residue at position 454.

7. An altered CYB5A protein that modifies 16-androstene steroid activity or production in pigs, said protein comprising:
a modification of the amino acids at one or more positions selected from the group consisting of: amino acids 21, 28, 52, 57, 62 or 70 of said porcine CYB5A protein compared to wild type (SEQ ID NO:2).

8. The altered CYB5A protein of claim 7 wherein said modification comprises one or more of:
(a) a methionine at position 52;
(b) an arginine at position 57;
(c) a serine at position 62;
(d) a serine at position 70;
(e) a lysine at position 21; and
(f) a valine at position 28.

9. A method of modifying 16-androstene steroid activity or production to reduce boar taint in a pig comprising;
introducing to said pig a polynucleotide that encodes a CYB5A protein of said pig, wherein amino acids at one or more positions selected from the group consisting of: amino acids 21, 28, 52, 57, 62 or 70 of the porcine CYB5A protein is modified from wild type (SEQ ID NO:2).

10. The method of claim 9 wherein said modification comprises one or more of:
(a) a methionine at position 52;
(b) an arginine at position 57;
(c) a serine at position 62;
(d) a serine at position 70;
(e) a lysine at position 21; and
(f) a valine at position 28.

11. The method of claim 9 further comprising introducing to said pig a polynucleotide that encodes a CYP17 protein of said pig, wherein amino acids at one or more positions selected from the group consisting of: amino acid 102, 103, 104, 106, 108, 112, 202, 344, 345, 348, 352 and 454 of the porcine CYP17 protein is modified from wild type (SEQ ID NO:4).

12. A genetically engineered pig having modified 16-androstene steroid activity said pig comprising:
a modified CYB5A protein, wherein said modification includes a change at amino acid 21, 28, 52, 57, 62 or 70 of the porcine CYB5A protein is modified from wild type (SEQ ID NO:2).

13. The genetically engineered pig of claim 12 wherein said modification comprises one or more of:
(a) a methionine at position 52;
(b) an arginine at position 57;
(c) a serine at position 62;
(d) a serine at position 70;
(e) a lysine at position 21; and
(f) a valine at position 28.

14. The genetically engineered pig of claim 12 further comprising a polynucleotide that encodes a CYP17 protein of said pig, wherein amino acids at one or more positions selected from the group consisting of: amino acid 102, 103, 104, 106, 108, 112, 202, 344, 345, 348, 352 and 454 of the porcine CYP17 protein is modified from wild type (SEQ ID NO:4).

15. A method of identifying animals to determine those with altered 16-androstene steroid synthesis and concomitant boar taint characteristics, comprising:
obtaining a sample of genetic material from said animal; and
assaying for the presence of a genotype in said animal which is associated with improved boar taint, said genotype characterized by one or more of the following:
a) a polymorphism in the CYP17 gene, said polymorphism resulting in a modification at one or more positions selected from the group consisting of: amino acid 102, 103, 104, 106, 108, 112, 202, 344, 345, 348, 352 and 454 of the porcine CYP17 protein; and
b) a polymorphism in the CYB5 gene, said polymorphism resulting in a modification at one or more positions selected from the group consisting of: amino acid 21, 28, 52, 57, 62 or 70 of said porcine CYB5A protein.

16. An isolated polynucleotide comprising a nucleotide sequence encoding an altered CYB5A protein with modified 16-androstene steroid activity or production in pigs, said protein comprising:
a modification of the amino acids at one or more positions selected from the group consisting of: amino acids 21, 28, 52, 57, 62 or 70 of said porcine CYB5A protein compared to wild type (SEQ ID NO:2).

17. The isolated polynucleotide of claim 16 wherein nucleotide sequence further comprises:
(a) a polynucleotide of SEQ ID NOs:23-31;
(b) a polynucleotide having at least 90% sequence identity across the entire sequence to SEQ ID NO:23-31;
(c) a polynucleotide amplified from a nucleic acid library using primers which selectively hybridize, under stringent hybridization conditions, to a sequence within a polynucleotide of SEQ ID NO:23-31; or
(d) a polynucleotide which is a full length complement of a polynucleotide of (a), (b), or (c).

18. An isolated polynucleotide comprising a nucleotide sequence encoding an altered CYP17 protein with modified 16-androstene steroid activity or production in pigs, said protein comprising:
a modification of the amino acid present at one or more positions selected from the group consisting of: amino acids 102, 103, 104, 106, 108, 109, 112, 202, 344, 345, 348, 352 and 454 of the porcine CYP17 protein, compared to wild type (SEQ ID NO:4).

19. The isolated polynucleotide of claim 16 wherein nucleotide sequence further comprises:
(a) a polynucleotide of SEQ ID NO:32-50;
(b) a polynucleotide having at least 90% sequence identity across the entire sequence to SEQ ID NO: 32-50;
(c) a polynucleotide amplified from a nucleic acid library using primers which selectively hybridize, under stringent hybridization conditions, to a sequence within a polynucleotide of SEQ ID NO: 32-50; or
(d) a polynucleotide which is a full length complement of a polynucleotide of (a), (b), or (c).
